SEQUENCE LISTING (206 sequences)

SEQ ID NO: 1 - protein, 4 aa, Artificial Sequence
  Exemplary minimal Furin cleavage consensus sequence
  misc_feature (2)..(2): Xaa can be any naturally occurring amino acid
RXKR

SEQ ID NO: 2 - protein, 4 aa, Artificial Sequence
  Exemplary minimal Furin cleavage consensus sequence
  misc_feature (2)..(2): Xaa can be any naturally occurring amino acid
RXRR

SEQ ID NO: 3 - protein, 4 aa, Artificial Sequence
  Exemplary minimal Furin cleavage consensus sequence
RRKR

SEQ ID NO: 4 - protein, 4 aa, Artificial Sequence
  Exemplary minimal Furin cleavage consensus sequence
RRRR

SEQ ID NO: 5 - protein, 4 aa, Artificial Sequence
  Exemplary minimal Furin cleavage consensus sequence
RSKR

SEQ ID NO: 6 - protein, 4 aa, Artificial Sequence
  Exemplary minimal Furin cleavage consensus sequence
RSRR

SEQ ID NO: 7 - protein, 4 aa, Artificial Sequence
  Exemplary minimal Furin cleavage consensus sequence
RKKR

SEQ ID NO: 8 - protein, 4 aa, Artificial Sequence
  Exemplary minimal Furin cleavage consensus sequence
RKRR

SEQ ID NO: 9 - protein, 4 aa, Artificial Sequence
  Exemplary minimal Furin cleavage consensus sequence
RQKR

SEQ ID NO: 10 - protein, 4 aa, Artificial Sequence
  Exemplary minimal Furin cleavage consensus sequence
RQRR

SEQ ID NO: 11 - protein, 4 aa, Artificial Sequence
  Exemplary minimal Furin cleavage consensus sequence
RTKR

SEQ ID NO: 12 - protein, 4 aa, Artificial Sequence
  Exemplary minimal Furin cleavage consensus sequence
RTRR

SEQ ID NO: 13 - protein, 4 aa, Artificial Sequence
  Exemplary minimal Furin cleavage consensus sequence
REKR

SEQ ID NO: 14 - protein, 4 aa, Artificial Sequence
  Exemplary minimal Furin cleavage consensus sequence
RERR

SEQ ID NO: 15 - protein, 4 aa, Artificial Sequence
  Exemplary minimal Furin cleavage consensus sequence
RDKR

SEQ ID NO: 16 - protein, 4 aa, Artificial Sequence
  Exemplary minimal Furin cleavage consensus sequence
RDRR

SEQ ID NO: 17 - protein, 4 aa, Artificial Sequence
  Exemplary minimal Furin cleavage consensus sequence
RHKR

SEQ ID NO: 18 - protein, 4 aa, Artificial Sequence
  Exemplary minimal Furin cleavage consensus sequence
RHRR

SEQ ID NO: 19 - protein, 4 aa, Artificial Sequence
  Exemplary minimal Furin cleavage consensus sequence
RFKR

SEQ ID NO: 20 - protein, 4 aa, Artificial Sequence
  Exemplary minimal Furin cleavage consensus sequence
RFRR

SEQ ID NO: 21 - protein, 4 aa, Artificial Sequence
  Exemplary minimal Furin cleavage consensus sequence
RAKR

SEQ ID NO: 22 - protein, 4 aa, Artificial Sequence
  Exemplary minimal Furin cleavage consensus sequence
RARR

SEQ ID NO: 23 - protein, 4 aa, Artificial Sequence
  Exemplary minimal Furin cleavage consensus sequence
RNKR

SEQ ID NO: 24 - protein, 4 aa, Artificial Sequence
  Exemplary minimal Furin cleavage consensus sequence
RNRR

SEQ ID NO: 25 - protein, 4 aa, Artificial Sequence
  Exemplary minimal Furin cleavage consensus sequence
RCKR

SEQ ID NO: 26 - protein, 4 aa, Artificial Sequence
  Exemplary minimal Furin cleavage consensus sequence
RCRR

SEQ ID NO: 27 - protein, 4 aa, Artificial Sequence
  Exemplary minimal Furin cleavage consensus sequence
RGKR

SEQ ID NO: 28 - protein, 4 aa, Artificial Sequence
  Exemplary minimal Furin cleavage consensus sequence
RGRR

SEQ ID NO: 29 - protein, 4 aa, Artificial Sequence
  Exemplary minimal Furin cleavage consensus sequence
RIKR

SEQ ID NO: 30 - protein, 4 aa, Artificial Sequence
  Exemplary minimal Furin cleavage consensus sequence
RIRR

SEQ ID NO: 31 - protein, 4 aa, Artificial Sequence
  Exemplary minimal Furin cleavage consensus sequence
RLKR

SEQ ID NO: 32 - protein, 4 aa, Artificial Sequence
  Exemplary minimal Furin cleavage consensus sequence
RLRR

SEQ ID NO: 33 - protein, 4 aa, Artificial Sequence
  Exemplary minimal Furin cleavage consensus sequence
RMKR

SEQ ID NO: 34 - protein, 4 aa, Artificial Sequence
  Exemplary minimal Furin cleavage consensus sequence
RMRR

SEQ ID NO: 35 - protein, 4 aa, Artificial Sequence
  Exemplary minimal Furin cleavage consensus sequence
RPKR

SEQ ID NO: 36 - protein, 4 aa, Artificial Sequence
  Exemplary minimal Furin cleavage consensus sequence
RPRR

SEQ ID NO: 37 - protein, 4 aa, Artificial Sequence
  Exemplary minimal Furin cleavage consensus sequence
RWKR

SEQ ID NO: 38 - protein, 4 aa, Artificial Sequence
  Exemplary minimal Furin cleavage consensus sequence
RWRR

SEQ ID NO: 39 - protein, 4 aa, Artificial Sequence
  Exemplary minimal Furin cleavage consensus sequence
RYKR

SEQ ID NO: 40 - protein, 4 aa, Artificial Sequence
  Exemplary minimal Furin cleavage consensus sequence
RYRR

SEQ ID NO: 41 - protein, 4 aa, Artificial Sequence
  Exemplary minimal Furin cleavage consensus sequence
RVKR

SEQ ID NO: 42 - protein, 4 aa, Artificial Sequence
  Exemplary minimal Furin cleavage consensus sequence
RVRR

SEQ ID NO: 43 - protein, 19 aa, Artificial Sequence
  P2A amino acid sequence (wild-type)
ATNFSLLKQAGDVEENPGP

SEQ ID NO: 44 - protein, 24 aa, Artificial Sequence
  F2A amino acid sequence (wild-type)
APVKQTLNFDLLKLAGDVESNPGP

SEQ ID NO: 45 - protein, 20 aa, Artificial Sequence
  E2A amino acid sequence (wild-type)
QCTNYALLKLAGDVESNPGP

SEQ ID NO: 46 - protein, 18 aa, Artificial Sequence
  T2A amino acid sequence (wild-type)
EGRGSLLTCGDVEENPGP

SEQ ID NO: 47 - DNA, 57 bp, Artificial Sequence
  P2A nucleotide sequence (wild-type)
gctactaacttcagcctgctgaagcaggctggagacgtggaggagaaccctgggccc

SEQ ID NO: 48 - DNA, 72 bp, Artificial Sequence
  F2A nucleotide sequence (wild-type)
gcaccggtgaaacagactttgaattttgaccttctgaagttggcaggagacgttgagtccaaccctgggccc

SEQ ID NO: 49 - DNA, 60 bp, Artificial Sequence
  E2A nucleotide sequence (wild-type)
cagtgtactaattatgctctcttgaaattggctggagatgttgagagcaaccctgggccc

SEQ ID NO: 50 - DNA, 54 bp, Artificial Sequence
  T2A nucleotide sequence (wild-type)
gagggcagaggaagtctgctaacatgcggtgacgtcgaggagaatcctgggccc

SEQ ID NO: 51 - protein, 5 aa, Artificial Sequence
  Exemplary P2A fragment sequence
ATNFS

SEQ ID NO: 52 - protein, 5 aa, Artificial Sequence
  Exemplary P2A fragment sequence with A1G mutation
GTNFS

SEQ ID NO: 53 - protein, 5 aa, Artificial Sequence
  Exemplary P2A fragment sequence with F4A mutation
ATNAS

SEQ ID NO: 54 - protein, 5 aa, Artificial Sequence
  Exemplary P2A fragment sequence with N3P mutation
ATPFS

SEQ ID NO: 55 - protein, 5 aa, Artificial Sequence
  Exemplary P2A fragment sequence with N3A mutation
ATAFS

SEQ ID NO: 56 - protein, 5 aa, Artificial Sequence
  Exemplary P2A fragment sequence with F4P mutation
ATNPS

SEQ ID NO: 57 - protein, 5 aa, Artificial Sequence
  Exemplary P2A fragment sequence with S5P mutation
ATNFP

SEQ ID NO: 58 - protein, 5 aa, Artificial Sequence
  Exemplary P2A fragment sequence with T2P mutation
APNFS

SEQ ID NO: 59 - protein, 5 aa, Artificial Sequence
  Exemplary P2A fragment sequence with T2G mutation
AGNFS

SEQ ID NO: 60 - protein, 5 aa, Artificial Sequence
  Exemplary P2A fragment sequence with A1P mutation
PTNFS

SEQ ID NO: 61 - protein, 9 aa, Artificial Sequence
  Exemplary Furin RRKR and P2A fragment sequence
RRKRATNFS

SEQ ID NO: 62 - protein, 9 aa, Artificial Sequence
  Exemplary Furin RRKR and P2A fragment sequence with A1G mutation
RRKRGTNFS

SEQ ID NO: 63 - protein, 9 aa, Artificial Sequence
  Exemplary Furin RRKR and P2A fragment sequence with F4A mutation
RRKRATNAS

SEQ ID NO: 64 - protein, 9 aa, Artificial Sequence
  Exemplary Furin RRKR and P2A fragment sequence with N3P mutation
RRKRATPFS

SEQ ID NO: 65 - protein, 9 aa, Artificial Sequence
  Exemplary Furin RRKR and P2A fragment sequence with N3A mutation
RRKRATAFS

SEQ ID NO: 66 - protein, 9 aa, Artificial Sequence
  Exemplary Furin RRKR and P2A fragment sequence with F4P mutation
RRKRATNPS

SEQ ID NO: 67 - protein, 9 aa, Artificial Sequence
  Exemplary Furin RRKR and P2A fragment sequence with S5P mutation
RRKRATNFP

SEQ ID NO: 68 - protein, 9 aa, Artificial Sequence
  Exemplary Furin RRKR and P2A fragment sequence with T2P mutation
RRKRAPNFS

SEQ ID NO: 69 - protein, 9 aa, Artificial Sequence
  Exemplary Furin RRKR and P2A fragment sequence with T2G mutation
RRKRAGNFS

SEQ ID NO: 70 - protein, 9 aa, Artificial Sequence
  Exemplary Furin RRKR and P2A fragment sequence with A1P mutation
RRKRPTNFS

SEQ ID NO: 71 - protein, 9 aa, Artificial Sequence
  Exemplary Furin RRRR and P2A fragment sequence
RRRRATNFS

SEQ ID NO: 72 - protein, 9 aa, Artificial Sequence
  Exemplary Furin RRRR and P2A fragment sequence with A1G mutation
RRRRGTNFS

SEQ ID NO: 73 - protein, 9 aa, Artificial Sequence
  Exemplary Furin RRRR and P2A fragment sequence with F4A mutation
RRRRATNAS

SEQ ID NO: 74 - protein, 9 aa, Artificial Sequence
  Exemplary Furin RRRR and P2A fragment sequence with N3P mutation
RRRRATPFS

SEQ ID NO: 75 - protein, 9 aa, Artificial Sequence
  Exemplary Furin RRRR and P2A fragment sequence with N3A mutation
RRRRATAFS

SEQ ID NO: 76 - protein, 9 aa, Artificial Sequence
  Exemplary Furin RRKR and P2A fragment sequence with F4P mutation
RRRRATNPS

SEQ ID NO: 77 - protein, 9 aa, Artificial Sequence
  Exemplary Furin RRRR and P2A fragment sequence with S5P mutation
RRRRATNFP

SEQ ID NO: 78 - protein, 9 aa, Artificial Sequence
  Exemplary Furin RRRR and P2A fragment sequence with T2P mutation
RRRRAPNFS

SEQ ID NO: 79 - protein, 9 aa, Artificial Sequence
  Exemplary Furin RRRR and P2A fragment sequence with T2G mutation
RRRRAGNFS

SEQ ID NO: 80 - protein, 9 aa, Artificial Sequence
  Exemplary Furin RRRR and P2A fragment sequence with A1P mutation
RRRRPTNFS

SEQ ID NO: 81 - protein, 9 aa, Artificial Sequence
  Exemplary Furin RXKR and P2A fragment sequence
  misc_feature (2)..(2): Xaa can be any naturally occurring amino acid
RXKRATNFS

SEQ ID NO: 82 - protein, 9 aa, Artificial Sequence
  Exemplary Furin RXKR and P2A fragment sequence with A1G mutation
  misc_feature (2)..(2): Xaa can be any naturally occurring amino acid
RXKRGTNFS

SEQ ID NO: 83 - protein, 9 aa, Artificial Sequence
  Exemplary Furin RXKR and P2A fragment sequence with F4A mutation
  misc_feature (2)..(2): Xaa can be any naturally occurring amino acid
RXKRATNAS

SEQ ID NO: 84 - protein, 9 aa, Artificial Sequence
  Exemplary Furin RXKR and P2A fragment sequence with N3P mutation
  misc_feature (2)..(2): Xaa can be any naturally occurring amino acid
RXKRATPFS

SEQ ID NO: 85 - protein, 9 aa, Artificial Sequence
  Exemplary Furin RXKR and P2A fragment sequence with N3A mutation
  misc_feature (2)..(2): Xaa can be any naturally occurring amino acid
RXKRATAFS

SEQ ID NO: 86 - protein, 9 aa, Artificial Sequence
  Exemplary Furin RXKR and P2A fragment sequence with F4P mutation
  misc_feature (2)..(2): Xaa can be any naturally occurring amino acid
RXKRATNPS

SEQ ID NO: 87 - protein, 9 aa, Artificial Sequence
  Exemplary Furin RXKR and P2A fragment sequence with S5P mutation
  misc_feature (2)..(2): Xaa can be any naturally occurring amino acid
RXKRATNFP

SEQ ID NO: 88 - protein, 9 aa, Artificial Sequence
  Exemplary Furin RXKR and P2A fragment sequence with T2P mutation
  misc_feature (2)..(2): Xaa can be any naturally occurring amino acid
RXKRAPNFS

SEQ ID NO: 89 - protein, 9 aa, Artificial Sequence
  Exemplary Furin RXKR and P2A fragment sequence with T2G mutation
  misc_feature (2)..(2): Xaa can be any naturally occurring amino acid
RXKRAGNFS

SEQ ID NO: 90 - protein, 9 aa, Artificial Sequence
  Exemplary Furin RXKR and P2A fragment sequence with A1P mutation
  misc_feature (2)..(2): Xaa can be any naturally occurring amino acid
RXKRPTNFS

SEQ ID NO: 91 - protein, 9 aa, Artificial Sequence
  Exemplary Furin RXRR and P2A fragment sequence
  misc_feature (2)..(2): Xaa can be any naturally occurring amino acid
RXRRATNFS

SEQ ID NO: 92 - protein, 9 aa, Artificial Sequence
  Exemplary Furin RXRR and P2A fragment sequence with A1G mutation
  misc_feature (2)..(2): Xaa can be any naturally occurring amino acid
RXRRGTNFS

SEQ ID NO: 93 - protein, 9 aa, Artificial Sequence
  Exemplary Furin RXRR and P2A fragment sequence with F4A mutation
  misc_feature (2)..(2): Xaa can be any naturally occurring amino acid
RXRRATNAS

SEQ ID NO: 94 - protein, 9 aa, Artificial Sequence
  Exemplary Furin RXRR and P2A fragment sequence with N3P mutation
  misc_feature (2)..(2): Xaa can be any naturally occurring amino acid
RXRRATPFS

SEQ ID NO: 95 - protein, 9 aa, Artificial Sequence
  Exemplary Furin RXRR and P2A fragment sequence with N3A mutation
  misc_feature (2)..(2): Xaa can be any naturally occurring amino acid
RXRRATAFS

SEQ ID NO: 96 - protein, 9 aa, Artificial Sequence
  Exemplary Furin RXKR and P2A fragment sequence with F4P mutation
  misc_feature (2)..(2): Xaa can be any naturally occurring amino acid
RXRRATNPS

SEQ ID NO: 97 - protein, 9 aa, Artificial Sequence
  Exemplary Furin RXRR and P2A fragment sequence with S5P mutation
  misc_feature (2)..(2): Xaa can be any naturally occurring amino acid
RXRRATNFP

SEQ ID NO: 98 - protein, 9 aa, Artificial Sequence
  Exemplary Furin RXRR and P2A fragment sequence with T2P mutation
  misc_feature (2)..(2): Xaa can be any naturally occurring amino acid
RXRRAPNFS

SEQ ID NO: 99 - protein, 9 aa, Artificial Sequence
  Exemplary Furin RXRR and P2A fragment sequence with T2G mutation
  misc_feature (2)..(2): Xaa can be any naturally occurring amino acid
RXRRAGNFS

SEQ ID NO: 100 - protein, 9 aa, Artificial Sequence
  Exemplary Furin RXRR and P2A fragment sequence with A1P mutation
  misc_feature (2)..(2): Xaa can be any naturally occurring amino acid
RXRRPTNFS

SEQ ID NO: 101 - protein, 19 aa, Artificial Sequence
  Exemplary P2A sequence with A1G mutation
GTNFSLLKQAGDVEENPGP

SEQ ID NO: 102 - protein, 19 aa, Artificial Sequence
  Exemplary P2A sequence with F4A mutation
ATNASLLKQAGDVEENPGP

SEQ ID NO: 103 - protein, 19 aa, Artificial Sequence
  Exemplary P2A sequence with N3P mutation
ATPFSLLKQAGDVEENPGP

SEQ ID NO: 104 - protein, 19 aa, Artificial Sequence
  Exemplary P2A sequence with N3A mutation
ATAFSLLKQAGDVEENPGP

SEQ ID NO: 105 - protein, 19 aa, Artificial Sequence
  Exemplary P2A sequence with F4P mutation
ATNPSLLKQAGDVEENPGP

SEQ ID NO: 106 - protein, 19 aa, Artificial Sequence
  Exemplary P2A sequence with S5P mutation
ATNFPLLKQAGDVEENPGP

SEQ ID NO: 107 - protein, 19 aa, Artificial Sequence
  Exemplary P2A sequence with T2P mutation
APNFSLLKQAGDVEENPGP

SEQ ID NO: 108 - protein, 19 aa, Artificial Sequence
  Exemplary P2A sequence with T2G mutation
AGNFSLLKQAGDVEENPGP

SEQ ID NO: 109 - protein, 19 aa, Artificial Sequence
  Exemplary P2A sequence with A1P mutation
PTNFSLLKQAGDVEENPGP

SEQ ID NO: 110 - protein, 23 aa, Artificial Sequence
  Exemplary Furin RRKR and P2A sequence
RRKRATNFSLLKQAGDVEENPGP

SEQ ID NO: 111 - protein, 23 aa, Artificial Sequence
  Exemplary Furin RRKR and P2A sequence with A1G mutation
RRKRGTNFSLLKQAGDVEENPGP

SEQ ID NO: 112 - protein, 23 aa, Artificial Sequence
  Exemplary Furin RRKR and P2A sequence with F4A mutation
RRKRATNASLLKQAGDVEENPGP

SEQ ID NO: 113 - protein, 23 aa, Artificial Sequence
  Exemplary Furin RRKR and P2A sequence with N3P mutation
RRKRATPFSLLKQAGDVEENPGP

SEQ ID NO: 114 - protein, 23 aa, Artificial Sequence
  Exemplary Furin RRKR and P2A sequence with N3A mutation
RRKRATAFSLLKQAGDVEENPGP

SEQ ID NO: 115 - protein, 23 aa, Artificial Sequence
  Exemplary Furin RRKR and P2A sequence with F4P mutation
RRKRATNPSLLKQAGDVEENPGP

SEQ ID NO: 116 - protein, 23 aa, Artificial Sequence
  Exemplary Furin RRKR and P2A sequence with S5P mutation
RRKRATNFPLLKQAGDVEENPGP

SEQ ID NO: 117 - protein, 23 aa, Artificial Sequence
  Exemplary Furin RRKR and P2A sequence with T2P mutation
RRKRAPNFSLLKQAGDVEENPGP

SEQ ID NO: 118 - protein, 23 aa, Artificial Sequence
  Exemplary Furin RRKR and P2A sequence with T2G mutation
RRKRAGNFSLLKQAGDVEENPGP

SEQ ID NO: 119 - protein, 23 aa, Artificial Sequence
  Exemplary Furin RRKR and P2A sequence with A1P mutation
RRKRPTNFSLLKQAGDVEENPGP

SEQ ID NO: 120 - protein, 23 aa, Artificial Sequence
  Exemplary Furin RRRR and P2A sequence
RRRRATNFSLLKQAGDVEENPGP

SEQ ID NO: 121 - protein, 23 aa, Artificial Sequence
  Exemplary Furin RRRR and P2A sequence with A1G mutation
RRRRGTNFSLLKQAGDVEENPGP

SEQ ID NO: 122 - protein, 23 aa, Artificial Sequence
  Exemplary Furin RRRR and P2A sequence with F4A mutation
RRRRATNASLLKQAGDVEENPGP

SEQ ID NO: 123 - protein, 23 aa, Artificial Sequence
  Exemplary Furin RRRR and P2A sequence with N3P mutation
RRRRATPFSLLKQAGDVEENPGP

SEQ ID NO: 124 - protein, 23 aa, Artificial Sequence
  Exemplary Furin RRRR and P2A sequence with N3A mutation
RRRRATAFSLLKQAGDVEENPGP

SEQ ID NO: 125 - protein, 23 aa, Artificial Sequence
  Exemplary Furin RRKR and P2A sequence with F4P mutation
RRRRATNPSLLKQAGDVEENPGP

SEQ ID NO: 126 - protein, 23 aa, Artificial Sequence
  Exemplary Furin RRRR and P2A sequence with S5P mutation
RRRRATNFPLLKQAGDVEENPGP

SEQ ID NO: 127 - protein, 23 aa, Artificial Sequence
  Exemplary Furin RRRR and P2A sequence with T2P mutation
RRRRAPNFSLLKQAGDVEENPGP

SEQ ID NO: 128 - protein, 23 aa, Artificial Sequence
  Exemplary Furin RRRR and P2A sequence with T2G mutation
RRRRAGNFSLLKQAGDVEENPGP

SEQ ID NO: 129 - protein, 23 aa, Artificial Sequence
  Exemplary Furin RRRR and P2A sequence with A1P mutation
RRRRPTNFSLLKQAGDVEENPGP

SEQ ID NO: 130 - protein, 23 aa, Artificial Sequence
  Exemplary Furin RXKR and P2A sequence
  misc_feature (2)..(2): Xaa can be any naturally occurring amino acid
RXKRATNFSLLKQAGDVEENPGP

SEQ ID NO: 131 - protein, 23 aa, Artificial Sequence
  Exemplary Furin RXKR and P2A sequence with A1G mutation
  misc_feature (2)..(2): Xaa can be any naturally occurring amino acid
RXKRGTNFSLLKQAGDVEENPGP

SEQ ID NO: 132 - protein, 23 aa, Artificial Sequence
  Exemplary Furin RXKR and P2A sequence with F4A mutation
  misc_feature (2)..(2): Xaa can be any naturally occurring amino acid
RXKRATNASLLKQAGDVEENPGP

SEQ ID NO: 133 - protein, 23 aa, Artificial Sequence
  Exemplary Furin RXKR and P2A sequence with N3P mutation
  misc_feature (2)..(2): Xaa can be any naturally occurring amino acid
RXKRATPFSLLKQAGDVEENPGP

SEQ ID NO: 134 - protein, 23 aa, Artificial Sequence
  Exemplary Furin RXKR and P2A sequence with N3A mutation
  misc_feature (2)..(2): Xaa can be any naturally occurring amino acid
RXKRATAFSLLKQAGDVEENPGP

SEQ ID NO: 135 - protein, 23 aa, Artificial Sequence
  Exemplary Furin RXKR and P2A sequence with F4P mutation
  misc_feature (2)..(2): Xaa can be any naturally occurring amino acid
RXKRATNPSLLKQAGDVEENPGP

SEQ ID NO: 136 - protein, 23 aa, Artificial Sequence
  Exemplary Furin RXKR and P2A sequence with S5P mutation
  misc_feature (2)..(2): Xaa can be any naturally occurring amino acid
RXKRATNFPLLKQAGDVEENPGP

SEQ ID NO: 137 - protein, 23 aa, Artificial Sequence
  Exemplary Furin RXKR and P2A sequence with T2P mutation
  misc_feature (2)..(2): Xaa can be any naturally occurring amino acid
RXKRAPNFSLLKQAGDVEENPGP

SEQ ID NO: 138 - protein, 23 aa, Artificial Sequence
  Exemplary Furin RXKR and P2A sequence with T2G mutation
  misc_feature (2)..(2): Xaa can be any naturally occurring amino acid
RXKRAGNFSLLKQAGDVEENPGP

SEQ ID NO: 139 - protein, 23 aa, Artificial Sequence
  Exemplary Furin RXKR and P2A sequence with A1P mutation
  misc_feature (2)..(2): Xaa can be any naturally occurring amino acid
RXKRPTNFSLLKQAGDVEENPGP

SEQ ID NO: 140 - protein, 23 aa, Artificial Sequence
  Exemplary Furin RXRR and P2A sequence
  misc_feature (2)..(2): Xaa can be any naturally occurring amino acid
RXRRATNFSLLKQAGDVEENPGP

SEQ ID NO: 141 - protein, 23 aa, Artificial Sequence
  Exemplary Furin RXRR and P2A sequence with A1G mutation
  misc_feature (2)..(2): Xaa can be any naturally occurring amino acid
RXRRGTNFSLLKQAGDVEENPGP

SEQ ID NO: 142 - protein, 23 aa, Artificial Sequence
  Exemplary Furin RXRR and P2A sequence with F4A mutation
  misc_feature (2)..(2): Xaa can be any naturally occurring amino acid
RXRRATNASLLKQAGDVEENPGP

SEQ ID NO: 143 - protein, 23 aa, Artificial Sequence
  Exemplary Furin RXRR and P2A sequence with N3P mutation
  misc_feature (2)..(2): Xaa can be any naturally occurring amino acid
RXRRATPFSLLKQAGDVEENPGP

SEQ ID NO: 144 - protein, 23 aa, Artificial Sequence
  Exemplary Furin RXRR and P2A sequence with N3A mutation
  misc_feature (2)..(2): Xaa can be any naturally occurring amino acid
RXRRATAFSLLKQAGDVEENPGP

SEQ ID NO: 145 - protein, 23 aa, Artificial Sequence
  Exemplary Furin RXKR and P2A sequence with F4P mutation
  misc_feature (2)..(2): Xaa can be any naturally occurring amino acid
RXRRATNPSLLKQAGDVEENPGP

SEQ ID NO: 146 - protein, 23 aa, Artificial Sequence
  Exemplary Furin RXRR and P2A sequence with S5P mutation
  misc_feature (2)..(2): Xaa can be any naturally occurring amino acid
RXRRATNFPLLKQAGDVEENPGP

SEQ ID NO: 147 - protein, 23 aa, Artificial Sequence
  Exemplary Furin RXRR and P2A sequence with T2P mutation
  misc_feature (2)..(2): Xaa can be any naturally occurring amino acid
RXRRAPNFSLLKQAGDVEENPGP

SEQ ID NO: 148 - protein, 23 aa, Artificial Sequence
  Exemplary Furin RXRR and P2A sequence with T2G mutation
  misc_feature (2)..(2): Xaa can be any naturally occurring amino acid
RXRRAGNFSLLKQAGDVEENPGP

SEQ ID NO: 149 - protein, 23 aa, Artificial Sequence
  Exemplary Furin RXRR and P2A sequence with A1P mutation
  misc_feature (2)..(2): Xaa can be any naturally occurring amino acid
RXRRPTNFSLLKQAGDVEENPGP

SEQ ID NO: 150 - protein, 60 aa, Artificial Sequence
  Amino acid sequence of  RRKR-P2A-GPI section in expression system
RRKRATNFSLLKQAGDVEENPGPPNKGSGTTSGTTRLLSGHTCFTLTGLLGTLVTMGLLT

SEQ ID NO: 151 - protein, 60 aa, Artificial Sequence; identical to SEQ ID NO: 150
  Amino acid sequence of RRKR-(ATNFS)P2A-GPI section in expression system

SEQ ID NO: 152 - protein, 46 aa, Artificial Sequence
  Amino acid sequence of RRKR-ATNFS-GPI section in expression system
RRKRATNFSPNKGSGTTSGTTRLLSGHTCFTLTGLLGTLVTMGLLT

SEQ ID NO: 153 - protein, 60 aa, Artificial Sequence
  Amino acid sequence of RRKR-(GTNFS)P2A-GPI section in expression system (comprises A1G mutation in P2A polypeptide)
RRKRGTNFSLLKQAGDVEENPGPPNKGSGTTSGTTRLLSGHTCFTLTGLLGTLVTMGLLT

SEQ ID NO: 154 - protein, 46 aa, Artificial Sequence
  Amino acid sequence of RRKR-GTNFS-GPI section in expression system (comprises A1G mutation in P2A polypeptide fragment)
RRKRGTNFSPNKGSGTTSGTTRLLSGHTCFTLTGLLGTLVTMGLLT

SEQ ID NO: 155 - protein, 60 aa, Artificial Sequence
  Amino acid sequence of RRKR-(ATNAS)P2A-GPI section in expression system (comprises F4A mutation in P2A polypeptide)
RRKRATNASLLKQAGDVEENPGPPNKGSGTTSGTTRLLSGHTCFTLTGLLGTLVTMGLLT

SEQ ID NO: 156 - protein, 46 aa, Artificial Sequence
  Amino acid sequence of RRKR-ATNAS-GPI section in expression system (comprises F4A mutation in P2A polypeptide fragment)
RRKRATNASPNKGSGTTSGTTRLLSGHTCFTLTGLLGTLVTMGLLT

SEQ ID NO: 157 - protein, 60 aa, Artificial Sequence
  Amino acid sequence of RRKR-(ATPFS)P2A-GPI section in expression system (comprises N3P mutation in P2A polypeptide)
RRKRATPFSLLKQAGDVEENPGPPNKGSGTTSGTTRLLSGHTCFTLTGLLGTLVTMGLLT

SEQ ID NO: 158 - protein, 46 aa, Artificial Sequence
  Amino acid sequence of RRKR-ATPFS-GPI section in expression system (comprises N3P mutation in P2A polypeptide fragment)
RRKRATPFSPNKGSGTTSGTTRLLSGHTCFTLTGLLGTLVTMGLLT

SEQ ID NO: 159 - protein, 60 aa, Artificial Sequence
  Amino acid sequence of RRKR-(ATAFS)P2A-GPI section in expression system (comprises N3A mutation in P2A polypeptide)
RRKRATAFSLLKQAGDVEENPGPPNKGSGTTSGTTRLLSGHTCFTLTGLLGTLVTMGLLT

SEQ ID NO: 160 - protein, 46 aa, Artificial Sequence
  Amino acid sequence of RRKR-ATAFS-GPI section in expression system (comprises N3A mutation in P2A polypeptide fragment)
RRKRATAFSPNKGSGTTSGTTRLLSGHTCFTLTGLLGTLVTMGLLT

SEQ ID NO: 161 - protein, 60 aa, Artificial Sequence
  Amino acid sequence of RRKR-(ATNPS)P2A-GPI section in expression system (comprises F4P mutation in P2A polypeptide)
RRKRATNPSLLKQAGDVEENPGPPNKGSGTTSGTTRLLSGHTCFTLTGLLGTLVTMGLLT

SEQ ID NO: 162 - protein, 46 aa, Artificial Sequence
  Amino acid sequence of RRKR-ATNPS-GPI section in expression system (comprises F4P mutation in P2A polypeptide fragment
RRKRATNPSPNKGSGTTSGTTRLLSGHTCFTLTGLLGTLVTMGLLT

SEQ ID NO: 163 - protein, 60 aa, Artificial Sequence
  Amino acid sequence of RRKR-(ATNFP)P2A-GPI section in expression system (comprises S5P mutation in P2A polypeptide)
RRKRATNFPLLKQAGDVEENPGPPNKGSGTTSGTTRLLSGHTCFTLTGLLGTLVTMGLLT

SEQ ID NO: 164 - protein, 46 aa, Artificial Sequence
  Amino acid sequence of RRKR-ATNFP-GPI section in expression system (comprises S5P mutation in P2A polypeptide fragment)
RRKRATNFPPNKGSGTTSGTTRLLSGHTCFTLTGLLGTLVTMGLLT

SEQ ID NO: 165 - protein, 60 aa, Artificial Sequence
  Amino acid sequence of RRKR-(APNFS)P2A-GPI section in expression system (comprises T2P mutation in P2A polypeptide)
RRKRAPNFSLLKQAGDVEENPGPPNKGSGTTSGTTRLLSGHTCFTLTGLLGTLVTMGLLT

SEQ ID NO: 166 - protein, 46 aa, Artificial Sequence
  Amino acid sequence of RRKR-APNFS-GPI section in expression system (comprises T2P mutation in P2A polypeptide fragment)
RRKRAPNFSPNKGSGTTSGTTRLLSGHTCFTLTGLLGTLVTMGLLT

SEQ ID NO: 167 - protein, 60 aa, Artificial Sequence
  Amino acid sequence of RRKR-(AGNFS)P2A-GPI section in expression system (comprises T2G mutation in P2A polypeptide)
RRKRAGNFSLLKQAGDVEENPGPPNKGSGTTSGTTRLLSGHTCFTLTGLLGTLVTMGLLT

SEQ ID NO: 168 - protein, 46 aa, Artificial Sequence
  Amino acid sequence of RRKR-AGNFS-GPI section in expression system (comprises T2G mutation in P2A polypeptide fragment)
RRKRAGNFSPNKGSGTTSGTTRLLSGHTCFTLTGLLGTLVTMGLLT

SEQ ID NO: 169 - protein, 60 aa, Artificial Sequence
  Amino acid sequence of RRKR-(PTNFS)P2A-GPI section in expression system (comprises A1P mutation in P2A polypeptide)
RRKRPTNFSLLKQAGDVEENPGPPNKGSGTTSGTTRLLSGHTCFTLTGLLGTLVTMGLLT

SEQ ID NO: 170 - protein, 46 aa, Artificial Sequence
  Amino acid sequence of RRKR-PTNFS-GPI section in expression system (comprises A1P mutation in P2A polypeptide fragment)
RRKRPTNFSPNKGSGTTSGTTRLLSGHTCFTLTGLLGTLVTMGLLT

SEQ ID NO: 171 - protein, 28 aa, Artificial Sequence
  RRKR-F2A (wild-type) amino acid sequence
RRKRAPVKQTLNFDLLKLAGDVESNPGP

SEQ ID NO: 172 - protein, 24 aa, Artificial Sequence
  RRKR-E2A (wild-type) amino acid sequence
RRKRQCTNYALLKLAGDVESNPGP

SEQ ID NO: 173 - protein, 22 aa, Artificial Sequence
  RRKR-T2A (wild-type) amino acid sequence
RRKREGRGSLLTCGDVEENPGP

SEQ ID NO: 174 - protein, 23 aa, Artificial Sequence
  RRKR-P2A (wild-type) amino acid sequence
RRKRATNFSLLKQAGDVEENPGP

SEQ ID NO: 175 - protein, 37 aa, Artificial Sequence
  GPI amino acid sequence
PNKGSGTTSGTTRLLSGHTCFTLTGLLGTLVTMGLLT

SEQ ID NO: 176 - DNA, 84 bp, Artificial Sequence
  RRKR-F2A (wild-type) nucleotide sequence
cggagaaagcgcgcaccggtgaaacagactttgaattttgaccttctgaagttggcaggagacgttgagtccaaccctgggccc

SEQ ID NO: 177 - DNA, 72 bp, Artificial Sequence
  RRKR-E2A (wild-type) nucleotide sequence
cggagaaagcgccagtgtactaattatgctctcttgaaattggctggagatgttgagagcaaccctgggccc

SEQ ID NO: 178 - DNA, 66 bp, Artificial Sequence
  RRKR-T2A (wild-type) nucleotide sequence
cggagaaagcgcgagggcagaggaagtctgctaacatgcggtgacgtcgaggagaatcctgggccc

SEQ ID NO: 179 - DNA, 69 bp, Artificial Sequence
  RRKR-P2A (wild-type) nucleotide sequence
cggagaaagcgcgctactaacttcagcctgctgaagcaggctggagacgtggaggagaaccctgggccc

SEQ ID NO: 180 - DNA, 111 bp, Artificial Sequence
  GPI nucleotide sequence
ccaaataaaggaagtggaaccacttcaggtactacccgtcttctatctgggcacacgtgtttcacgttgacaggtttgcttgggacgctagtaaccatgggcttgctgact

SEQ ID NO: 181 - DNA, 587 bp, Artificial Sequence
  IRES nucleotide sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaacgcgtatgataatatggccacaacc

SEQ ID NO: 182 - DNA, 1603 bp, Artificial Sequence
  ChiP nucleotide sequence
agtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgactagtcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtc

SEQ ID NO: 183 - protein, 46 aa, Artificial Sequence
  Amino acid sequence of RXKR-PTNFS-GPI (comprises A1P mutation in P2A polypeptide fragment)
  misc_feature (2)..(2): Xaa can be any naturally occurring amino acid
RXKRPTNFSPNKGSGTTSGTTRLLSGHTCFTLTGLLGTLVTMGLLT

SEQ ID NO: 184 - protein, 46 aa, Artificial Sequence
  Amino acid sequence of RXKR-GTNFS-GPI (comprises A1G mutation in P2A polypeptide fragment)
  misc_feature (2)..(2): Xaa can be any naturally occurring amino acid
RXKRGTNFSPNKGSGTTSGTTRLLSGHTCFTLTGLLGTLVTMGLLT

SEQ ID NO: 185 - protein, 46 aa, Artificial Sequence
  Amino acid sequence of RXKR-AGNFS-GPI (comprises T2G mutation in P2A polypeptide fragment)
  misc_feature (2)..(2): Xaa can be any naturally occurring amino acid
RXKRAGNFSPNKGSGTTSGTTRLLSGHTCFTLTGLLGTLVTMGLLT

SEQ ID NO: 186 - protein, 46 aa, Artificial Sequence
  Amino acid sequence of RXKR-APNFS-GPI (comprises T2P mutation in P2A polypeptide fragment)
  misc_feature (2)..(2): Xaa can be any naturally occurring amino acid
RXKRAPNFSPNKGSGTTSGTTRLLSGHTCFTLTGLLGTLVTMGLLT

SEQ ID NO: 187 - protein, 60 aa, Artificial Sequence
  Amino acid sequence of RXKR-(PTNFS)P2A-GPI (comprises A1P mutation in P2A polypeptide)
  misc_feature (2)..(2): Xaa can be any naturally occurring amino acid
RXKRPTNFSLLKQAGDVEENPGPPNKGSGTTSGTTRLLSGHTCFTLTGLLGTLVTMGLLT

SEQ ID NO: 188 - protein, 60 aa, Artificial Sequence
  Amino acid sequence of RXKR-(GTNFS)P2A-GPI (comprises A1G mutation in P2A polypeptide)
  misc_feature (2)..(2): Xaa can be any naturally occurring amino acid
RXKRGTNFSLLKQAGDVEENPGPPNKGSGTTSGTTRLLSGHTCFTLTGLLGTLVTMGLLT

SEQ ID NO: 189 - protein, 60 aa, Artificial Sequence
  Amino acid sequence of RXKR-(AGNFS)P2A-GPI (comprises T2G mutation in P2A polypeptide)
  misc_feature (2)..(2): Xaa can be any naturally occurring amino acid
RXKRAGNFSLLKQAGDVEENPGPPNKGSGTTSGTTRLLSGHTCFTLTGLLGTLVTMGLLT

SEQ ID NO: 190 - protein, 60 aa, Artificial Sequence
  Amino acid sequence of RXKR-(APNFS)P2A-GPI (comprises T2P mutation in P2A polypeptide)
  misc_feature (2)..(2): Xaa can be any naturally occurring amino acid
RXKRAPNFSLLKQAGDVEENPGPPNKGSGTTSGTTRLLSGHTCFTLTGLLGTLVTMGLLT

SEQ ID NO: 191 - protein, 46 aa, Artificial Sequence
  Amino acid sequence of RXRR-PTNFS-GPI (comprises A1P mutation in P2A polypeptide fragment)
  misc_feature (2)..(2): Xaa can be any naturally occurring amino acid
RXRRPTNFSPNKGSGTTSGTTRLLSGHTCFTLTGLLGTLVTMGLLT

SEQ ID NO: 192 - protein, 46 aa, Artificial Sequence
  Amino acid sequence of RXRR-GTNFS-GPI (comprises A1G mutation in P2A polypeptide fragment)
  misc_feature (2)..(2): Xaa can be any naturally occurring amino acid
RXRRGTNFSPNKGSGTTSGTTRLLSGHTCFTLTGLLGTLVTMGLLT

SEQ ID NO: 193 - protein, 46 aa, Artificial Sequence
  Amino acid sequence of RXRR-AGNFS-GPI (comprises T2G mutation in P2A polypeptide fragment)
  misc_feature (2)..(2): Xaa can be any naturally occurring amino acid
RXRRAGNFSPNKGSGTTSGTTRLLSGHTCFTLTGLLGTLVTMGLLT

SEQ ID NO: 194 - protein, 46 aa, Artificial Sequence
  Amino acid sequence of RXRR-APNFS-GPI (comprises T2P mutation in P2A polypeptide fragment)
  misc_feature (2)..(2): Xaa can be any naturally occurring amino acid
RXRRAPNFSPNKGSGTTSGTTRLLSGHTCFTLTGLLGTLVTMGLLT

SEQ ID NO: 195 - protein, 60 aa, Artificial Sequence
  Amino acid sequence of RXRR-(PTNFS)P2A-GPI (comprises A1P mutation in P2A polypeptide)
  misc_feature (2)..(2): Xaa can be any naturally occurring amino acid
RXRRPTNFSLLKQAGDVEENPGPPNKGSGTTSGTTRLLSGHTCFTLTGLLGTLVTMGLLT

SEQ ID NO: 196 - protein, 60 aa, Artificial Sequence
  Amino acid sequence of RXRR-(GTNFS)P2A-GPI (comprises A1G mutation in P2A polypeptide)
  misc_feature (2)..(2): Xaa can be any naturally occurring amino acid
RXRRGTNFSLLKQAGDVEENPGPPNKGSGTTSGTTRLLSGHTCFTLTGLLGTLVTMGLLT

SEQ ID NO: 197 - protein, 60 aa, Artificial Sequence
  Amino acid sequence of RXRR-(AGNFS)P2A-GPI (comprises T2G mutation in P2A polypeptide)
  misc_feature (2)..(2): Xaa can be any naturally occurring amino acid
RXRRAGNFSLLKQAGDVEENPGPPNKGSGTTSGTTRLLSGHTCFTLTGLLGTLVTMGLLT

SEQ ID NO: 198 - protein, 60 aa, Artificial Sequence
  Amino acid sequence of RXRR-(APNFS)P2A-GPI (comprises T2P mutation in P2A polypeptide)
  misc_feature (2)..(2): Xaa can be any naturally occurring amino acid
RXRRAPNFSLLKQAGDVEENPGPPNKGSGTTSGTTRLLSGHTCFTLTGLLGTLVTMGLLT

SEQ ID NO: 199 - protein, 46 aa, Artificial Sequence
  Amino acid sequence of RRRR-PTNFS-GPI (comprises A1P mutation in P2A polypeptide fragment)
RRRRPTNFSPNKGSGTTSGTTRLLSGHTCFTLTGLLGTLVTMGLLT

SEQ ID NO: 200 - protein, 46 aa, Artificial Sequence
  Amino acid sequence of RRRR-GTNFS-GPI (comprises A1G mutation in P2A polypeptide fragment)
RRRRGTNFSPNKGSGTTSGTTRLLSGHTCFTLTGLLGTLVTMGLLT

SEQ ID NO: 201 - protein, 46 aa, Artificial Sequence
  Amino acid sequence of RRRR-AGNFS-GPI (comprises T2G mutation in P2A polypeptide fragment)
RRRRAGNFSPNKGSGTTSGTTRLLSGHTCFTLTGLLGTLVTMGLLT

SEQ ID NO: 202 - protein, 46 aa, Artificial Sequence
  Amino acid sequence of RRRR-APNFS-GPI (comprises T2P mutation in P2A polypeptide fragment)
RRRRAPNFSPNKGSGTTSGTTRLLSGHTCFTLTGLLGTLVTMGLLT

SEQ ID NO: 203 - protein, 60 aa, Artificial Sequence
  Amino acid sequence of RRRR-(PTNFS)P2A-GPI (comprises A1P mutation in P2A polypeptide)
RRRRPTNFSLLKQAGDVEENPGPPNKGSGTTSGTTRLLSGHTCFTLTGLLGTLVTMGLLT

SEQ ID NO: 204 - protein, 60 aa, Artificial Sequence
  Amino acid sequence of RRRR-(GTNFS)P2A-GPI (comprises A1G mutation in P2A polypeptide)
RRRRGTNFSLLKQAGDVEENPGPPNKGSGTTSGTTRLLSGHTCFTLTGLLGTLVTMGLLT

SEQ ID NO: 205 - protein, 60 aa, Artificial Sequence
  Amino acid sequence of RRRR-(AGNFS)P2A-GPI (comprises T2G mutation in P2A polypeptide)
RRRRAGNFSLLKQAGDVEENPGPPNKGSGTTSGTTRLLSGHTCFTLTGLLGTLVTMGLLT

SEQ ID NO: 206 - protein, 60 aa, Artificial Sequence
  Amino acid sequence of RRRR-(APNFS)P2A-GPI (comprises T2P mutation in P2A polypeptide)
RRRRAPNFSLLKQAGDVEENPGPPNKGSGTTSGTTRLLSGHTCFTLTGLLGTLVTMGLLT

(19) United States
(12) Patent Application Publication
(10) Pub. No.: US 2024/0010752 A1
(43) Pub. Date: Jan. 11, 2024

(54) Expression System for Protein Production and Screening

(71) Applicant: AGENCY FOR SCIENCE, TECHNOLOGY AND RESEARCH, Singapore (SG)

(72) Inventors: Yuan Sheng Yang, Singapore (SG); Hwee Ming Jessna Yeo, Singapore (SG); Mariati Mariati, Singapore (SG); Shiyi Goh Fang, Singapore (SG)

(21) Appl. No.: 18/025,437

(22) PCT Filed: Oct. 1, 2021
(86) PCT No.: PCT/SG2021/050592

§ 371 Date: Mar. 9, 2023

(30) Foreign Application Priority Data
Oct. 2, 2020 (SG) 10202009841Y

(51) Int. Cl. C07K 16/46; G01N 33/68; C07K 14/71; C07K 14/705

(52) U.S. Cl. CPC C07K 16/468; G01N 33/6854; C07K 14/71; C07K 14/70532; C07K 2319/03

(57) ABSTRACT

Disclosed is an expression system for an antigen binding molecule and the uses thereof.

### CROSS-REFERENCE TO RELATED APPLICATIONS

This application claims the benefit of priority of Singapore patent application No. 10202009841Y, filed 2 Oct. 2020, the contents of it being hereby incorporated by reference in its entirety for all purposes.

### FIELD OF THE INVENTION

The present invention generally relates to the field of biotechnology in particular to a nucleotide based expression system. In particular, the present invention relates to an expression system for antigen binding molecules for dual screening purposes.

### BACKGROUND OF THE INVENTION

Antigen binding molecules such as antibodies are one of the fastest growing class of biotherapeutic molecules. One such example of antibodies is immunoglobulin G (IgG), which consists of two heavy chain (HC) and two light chain (LC) polypeptides.

Developing therapeutic antibodies remains a technically challenging, extremely time-consuming and costly process. The process of developing the correct antibodies is often unsuccessful as the antibodies might be ineffective or simply comprise the incorrect sequences, therefore resulting in a high attrition rate and a high risk of failure. The process of developing therapeutic antibodies starts with discovery of antibodies with specific binding affinity and desirable functionalities, followed by production of antibodies in mammalian cells to provide enough high-quality materials for preclinical and clinical studies before commercialization. Antibody discovery has relied on either in vivo immunization of animals or in vitro display-based technologies, such as phage display, bacterial display and mammalian cell display. Chinese hamster ovary cells (CHO) are the dominant mammalian cells for antibody production due to their capability to perform proper assembly of complexes and human-like glycosylation. The transition from the antibody development to the production, which involves re-cloning of antibody genes and changes in antibody format and production host cells, not only results in increased time and cost, but also failure of many antibody candidates.

In view of the above, there is a need to provide an expression system that permits screening of proteins, to enable the production of antigen binding molecules such as antibodies, and to keep the production of incorrectly processed antibody species to the minimum.

### SUMMARY

In one aspect, the present disclosure refers to an expression system for an antigen binding molecule, wherein the antigen binding molecule is either secretable or membrane-bound, comprising:
a first antigen binding polynucleotide encoding a first part of the antigen binding molecule;
a cleavage polynucleotide encoding a cleavage site comprising a Furin consensus sequence RXKR (SEQ ID NO: 1) or RXRR (SEQ ID NO: 2), and a 2A polypeptide fragment thereof;
an anchor polynucleotide encoding a membrane anchor polypeptide;
   wherein the 2A polypeptide fragment thereof comprises one or more mutations in any one of the amino acid residue to control the cleavage efficiencies of the cleavage site to modulate ratio of production of the secretable antigen binding molecule versus the membrane-bound antigen binding molecule;
   wherein the cleavage polynucleotide is in between the first antigen binding polynucleotide and the anchor polynucleotide;
   wherein when the cleavage site is cleaved, the secretable antigen binding molecule comprising the first part of the antigen binding molecule is released;
   wherein when the cleavage site is not cleaved, the membrane-bound antigen binding molecule comprising the first part of the antigen binding molecule, the Furin consensus sequence RXKR (SEQ ID NO: 1) or RXRR (SEQ ID NO: 2), the 2A polypeptide fragment thereof, and the membrane anchor polypeptide, is released.

In another aspect, the present disclosure refers to an expression system for an antigen binding molecule, wherein the antigen binding molecule is either secretable or membrane-bound, comprising:
a first antigen binding polynucleotide encoding a first part of the antigen binding molecule;
a first cleavage polynucleotide encoding a first cleavage site, wherein the first cleavage site is a minimal Furin cleavage consensus sequence RXKR (SEQ ID NO: 1) or RXRR (SEQ ID NO: 2);
a second cleavage polynucleotide encoding a self-processing second cleavage site, wherein the self-processing second cleavage site is a 2A polypeptide or a fragment thereof;
an anchor polynucleotide encoding a membrane anchor polypeptide;
   wherein the 2A polypeptide or the fragment thereof comprises one or more mutations in any one of the amino acid residue to control the cleavage efficiencies of the first cleavage site and the second cleavage site to modulate ratio of production of the secretable antigen binding molecule versus the membrane-bound antigen binding molecule;
   wherein the first and second cleavage polynucleotides are in between the first antigen binding polynucleotide and the anchor polynucleotide;
   wherein when the first cleavage site is cleaved, the secretable antigen binding molecule comprising the first part of the antigen binding molecule is released;
   wherein when the first and second cleavage sites are not cleaved, the membrane-bound antigen binding molecule comprising the first part of the antigen binding molecule, the minimal Furin cleavage consensus sequence RXKR (SEQ ID NO: 1) or RXRR (SEQ ID NO: 2), the 2A polypeptide or a fragment thereof, and the membrane anchor polypeptide, is released.

In another aspect, the present disclosure refers to a vector comprising the expression system as disclosed herein.

In another aspect, the present disclosure refers to a host cell comprising the expression system or the vector as disclosed herein.

In another aspect, the present disclosure refers to a kit comprising the expression system, the vector, or the host cell as disclosed herein.

In yet another aspect, the present disclosure refers to a method for detecting the presence of one or more secreted antibodies and/or one or more surface-bound antibodies in a sample, the method comprising:
providing an expression system as disclosed herein;
delivering said expression system to one or more target cells, wherein said target cell transcribes said expression system, wherein once transcribed, said cleavage site is cleaved in a first plurality of first part of the antigen binding molecule, so that said first plurality of first part of the antigen binding molecule do not comprise said membrane anchor polypeptide, and are thereby secreted by said target cell, and wherein said cleavage sites are not cleaved in a second plurality of first part of the antigen binding molecule, so that said second plurality of first part of the antigen binding molecule comprise said membrane anchor polypeptide, and are thereby bound to the surface of said target cell; and
detecting the presence or absence of said first plurality of first part of the antigen binding molecule secreted by said target cell and/or detecting a quantity of said second plurality of first part of the antigen binding molecule on the surface of said target cell.

In yet another aspect, the present disclosure refers to a method for detecting the presence of one or more secreted antibodies and/or one or more surface-bound antibodies in a sample, the method comprising:
providing an expression system as disclosed herein;
delivering said expression system to one or more target cells, wherein said target cell transcribes said expression system, wherein once transcribed, said first cleavage site is cleaved in a first plurality of first part of the antigen binding molecule, so that said first plurality of first part of the antigen binding molecule do not comprise said membrane anchor polypeptide, and are thereby secreted by said target cell, and wherein said first and second cleavage sites are not cleaved in a second plurality of first part of the antigen binding molecule, so that said second plurality of first part of the antigen binding molecule comprise said membrane anchor polypeptide, and are thereby bound to the surface of said target cell; and
detecting the presence or absence of said first plurality of first part of the antigen binding molecule secreted by said target cell and/or detecting a quantity of said second plurality of first part of the antigen binding molecule on the surface of said target cell.

In yet another aspect, the present disclosure refers to the expression system, the vector, the host cell or the kit as disclosed herein for use in screening antibody libraries or antibody production

### BRIEF DESCRIPTION OF THE DRAWINGS

The invention will be better understood with reference to the detailed description when considered in conjunction with the non-limiting examples and the accompanying drawings, in which:

FIG. 1 shows the schematic of the exemplary expression systems and uses thereof.

FIG. 1A is a schematic illustrating the exemplary expression systems as disclosed herein. FIG. 1B is a schematic illustrating the platform for accelerated antibody development and production by combining antibody display and production in exemplary CHO cells.

FIG. 2 shows the workflow for generation of an exemplary CHO master clone containing a tagging vector integrated into a single integration site. ChiP is a chimeric promoter consisting of murine cytomegalovirus (CMV) enhancer, human CMV core promoter and human CMV intron A; mCMV is a murine CMV enhancer and promoter; IRESv18 is a mutated encephalomyocarditis virus (EMCV) internal ribosome entry site (IRES); pA is a simian virus 40 (SV40) polyadenylation signal; Fw and F3 are Wild-type and mutated flippase recognition target sites respectively; EGFP is a cDNA encoding enhanced green fluorescence protein; Zeo is a zeocin resistant Sh ble gene cDNA; (-ATG)Pur is a puromycin N-acetyl-transferase cDNA without start codon; HYG is a hygromycin resistant gene cDNA; Flpe is an enhanced flippase recombinase cDNA.

FIG. 3 shows the validation of the exemplary CHO K1 master clones for integration of one copy of gene per cell. FIG. 3A is a schematic illustrating the overview for recombinase-mediated cassette exchange (RMCE) for expressing recombinant proteins. FIG. 3B is a schematic representation of targeting vectors. FIG. 3C shows the graphs of fluorescence-activated cell sorting (FACS) analysis of targeted pools generated by transfection of pTarget-DsRed alone, pTarget-EGFP clone or co-transfection of pTarget-DsRed and pTarget-EGFP. ChiP is a chimeric promoter consisting of murine cytomegalovirus (CMV) enhancer, human CMV core promoter and human CMV intron A; mCMV is a murine CMV enhancer and promoter; IRES is a wild-type encephalomyocarditis virus (EMCV) internal ribosome entry site (IRES); pA is a simian virus 40 (SV40) polyadenylation signal; Fw and F3 are Wild-type and mutated flippase recognition target sites respectively; EGFP is a cDNA encoding enhanced green fluorescence protein; DsRed is a cDNA encoding fluorescence protein DsRed; (-ATG)Pur is a puromycin N-acetyl-transferase cDNA with start codon removed; HYG is a hygromycin resistant gene cDNA; Flpe is a enhanced flippase recombinase cDNA; GOI is a gene of interest.

FIG. 4 shows the characterization of the exemplary CHO K1 master clones for antibody expression. FIG. 4A is a schematic representation of targeting vector carrying DsRed, LC and HC. IRES is a wild-type encephalomyocarditis virus (EMCV) internal ribosome entry site (IRES); Fw and F3 are Wild-type and mutated flippase recognition target sites respectively; DsRed is a cDNA encoding fluorescence protein DsRed; (-ATG)Pur is a puromycin N-acetyl-transferase cDNA with start codon removed; LC is a light chain cDNA; HC is a heavy chain cDNA. FIG. 4B are graphs showing fluorescence-activated cell sorting (FACS) analysis of targeted pools generated by transfection of the master clone with the targeting vector and the vector expressing Flpe. FIG. 4C shows graphs that shows the characterization of targeted pools for growth and titer in 14-day fed-batch cultures.

FIG. 5 shows the simultaneous display and secretion of antigen binding molecules, for example, IgG antibodies, from targeting vectors with HC directly linked to GPI or through furin cleavage sequence (RRKR (SEQ ID NO: 3), various 2A peptides or RRKR (SEQ ID NO: 3)-2A combinations. FIG. 5A is a schematic representation of an overview of recombinase-mediated cassette exchange (RMCE) and vector design for simultaneous display and secretion of IgG antibodies in targeted cells. FIG. 5B is a schematic representation of an overview of various targeting vectors. ChiP is a chimeric promoter consisting of murine cytomegalovirus (CMV) enhancer, human CMV core promoter and human CMV intron A; mCMV is a murine CMV enhancer and promoter; IRES is a wild type encephalomyocarditis virus (EMCV) internal ribosome entry site (IRES); pA is a simian virus 40 (SV40) polyadenylation signal; Fw and F3 are Wild-type and mutated flippase recognition target sites respectively; HYG is a hygromycin resistant gene cDNA; (-ATG)Pur is a puromycin N-acetyl-transferase cDNA with start codon removed; Flpe is an enhanced flippase recombinase cDNA; LC is a light chain cDNA; HC is a heavy chain cDNA; RRKR (SEQ ID NO: 3) is an exemplary furin recognition sequence; F2A is DNA encoding a 2A peptide foot-and-mouth disease virus; E2A is a DNA encoding a 2A peptide equine rhinitis A virus; T2A is a DNA encoding a 2A peptide Thosea asigna virus; P2A is a DNA encoding a 2A peptide derived from porcine teschovirus-1; GPI is a DNA encoding the glycosidylphosphatidylinositol membrane anchor derived from human decay-accelerating factor. FIG. 5C has graphs that shows the characterization of targeted cells generated using various targeting vectors for display and secretion of IgG antibodies. Each targeted pool of cells was generated using a specific targeting vector. The targeted cells were stained with anti-human IgG (y-chain specific) FITC conjugate for quantification of bound antibodies on the cell surface. The intensity of bound molecules were quantified as geometric mean fluorescene intensity (GMFI) using fluorescence-activated cell sorting (FACS). Each point in the figure represent the GMFI normalized to the GPI vector. The concentration of secreted antibody in the culture supernatant was quantified using Nephometer. The specific productivity for each vector is calculated as the antibody titer determined at day 7 divided by the corresponding integrated viable cell density. Each point in the figure represents the normalized specific productivity to the control vector. The secreted antibody was also analyzed using SDS PAGE under reducing conditions.

FIG. 6 shows the point mutation of P2A to control the ratios of membrane bound to secreted IgG antibodies. FIG. 6A is a schematic representation of targeting vectors with HC linked to a membrane anchor GPI through P2A or RRKR (SEQ ID NO: 3)-P2A. P2A has 19 amino acids (SEQ ID NO. 43). FIG. 6B shows graphs using P2A-GPI and RRKR-P2A-GPI vectors containing different P2A variants P2A variants generated by point mutation of each amino acid in P2A to glycine (G). FIG. 6C shows graphs using P2A-GPI and RRKR-P2A-GPI vectors containing different P2A variants P2A variants generated by point mutation of each amino acid in P2A to proline (P). FIG. 6D shows graphs using P2A-GPI and RRKR-P2A-GPI vectors containing different P2A variants P2A variants generated by point mutation of each amino acid in P2A to alanine (A). The graphs in FIGS. 6B-6D show the characterization of targeted cells generated using P2A-GPI and RRKR-P2A-GPI vectors containing different P2A variants for display and secretion of IgG antibodies. Each targeted pool of cells was generated using a targeting vector containing a specific P2A variant. The targeted cells were stained with anti-human IgG (γ-chain specific) FITC conjugate for quantification of bound antibodies on the cell surface. The intensity of bound molecules were quantified as geometric mean fluorescene intensity (GMFI) using fluorescence-activated cell sorting (FACS). Each point in the figure represents the GMFI normalized to the GM vector. The concentration of secreted antibody in the culture supernatant was quantified using Nephometer. The specific productivity for each vector is calculated as the antibody titer determined at day 7 divided by the corresponding integrated viable cell density. Each point in the figure represents the normalized specific productivity to the control vector. The photos in FIGS. 6B-6D are the Western Blot analysis of the secreted antibody using SDS PAGE under reducing conditions.

FIG. 7 shows the furin recognition sequence for controlling the ratios of membrane bound to secreted IgG antibodies. FIG. 7A is a schematic representation of targeting vectors with HC linked to a membrane anchor GPI through furin recognition sequence variants. The targeting vectors are named based on the section from the furin sequence to the membrane anchor GPI. The section from the furin sequence to the membrane anchor GPI can be identified by SEQ ID NOs: 150-170. FIG. 7B are graphs showing the characterization of targeted cells generated using vectors containing different furin variants for display and secretion of IgG antibodies. The targeted cells were stained with anti-human IgG (γ-chain specific) FITC conjugate for quantification of bound antibodies on the cell surface. The intensity of bound molecules were quantified as geometric mean fluorescene intensity (GMFI) using fluorescence-activated cell sorting (FACS). Each point in the figure represent the GMFI normalized to the GM vector. The concentration of secreted antibody in the culture supernatant was quantified using Nephometer. The specific productivity for each vector is calculated as the antibody titer determined at day 7 divided by the corresponding integrated viable cell density. Each point in the figure represents the normalized specific productivity to the control vector. The photo is the Western Blot analysis of the secreted antibody using SDS PAGE under reducing conditions.

FIG. 8 shows the application of simultaneous display and secretion system for antibody humanization. FIG. 8A is a schematic representation of the process of the design of variable light and heavy chain libraries into the expression system, and the production of the secretable and membrane-bound antigen binding molecules or antibodies. FIG. 8B is a schematic representation of the process of sorting the secretable and membrane-bound antigen binding molecules or antibodies during upscaled generation of the antigen binding molecules or antibodies prior to testing the antigen binding molecules or antibodies for binding affinity, immunogenicity and/or function.

FIG. 9 shows the DNA and amino acid sequences of various furin recognition sequence RRKR-2A peptides and GPI membrane anchor. From top to bottom: DNA sequence of RRKR-F2A (SEQ ID NO: 176); amino acid sequence of RRKR-F2A (SEQ ID NO: 171); DNA sequence of RRKR-E2A (SEQ ID NO: 177); amino acid sequence of RRKR-E2A (SEQ ID NO: 172); DNA sequence of RRKR-T2A (SEQ ID NO: 178); amino acid sequence of RRKR-T2A (SEQ ID NO: 173); DNA sequence of RRKR-P2A (SEQ ID NO: 179); amino acid sequence of RRKR-P2A (SEQ ID NO: 174); DNA sequence of GPI (SEQ ID NO: 180); amino acid sequence of GPI (SEQ ID NO: 175). F2A is a 2A peptide derived from foot-and-mouth disease virus; E2A is a 2A peptide derived from equine rhinitis A virus; T2A is a 2A peptide derived from Thosea asigna virus; P2A is a 2A peptide derived from porcine teschovirus-1; GPI is the glycosidylphosphatidylinositol membrane anchor derived from human decay-accelerating factor.

### DETAILED DESCRIPTION

Secreted recombinant IgGs are practical for production purposes, while cell-surface displayed antibodies are useful for screening/ sorting via FACS. The expression systems in use today show either high levels of membrane antibodies and little to no secretable antibodies, or vice versa, low levels of membrane antibodies and high levels secretable antibodies. Such unbalanced levels of expression of membrane and secretable antibodies cannot satisfy the need for a smooth transition from antibody discovery to production, and ultimately leads to an inefficient system for antibody screening and/or production.

In view of the above problems, there is a need to provide a more effective expression system that permits simultaneous cell surface display and secretion of the same protein at optimal ratios through the use of engineered peptides with different cleavage sites with different cleavage efficiencies. Such an expression system can be used for dual screening purpose and for more efficient antibody production. The inventors of the present disclosure have found expression systems for an antigen binding molecule, wherein the antigen binding molecule is either secretable or membrane-bound. The term “expression system” used herein refers to DNA construct that are designed to produce a protein, or an RNA (ribonucleic acid), either inside or outside a cell. The expression system can exist on its own or be incorporated into a vector. Exemplary expression systems include, but are not limited to, mammalian expression system, insect expression system, yeast expression system, bacteria expression system, algae expression system or a cell-free expression system. The expression system can comprise the following components including, but not limited to, a promoter, one or more genes of interest, one or more identification tags. In one example, expression system can be used for dual screening purpose to screen for the expression levels of the secretable and/or membrane-bound antigen binding molecule.

The term “antigen binding molecule” used herein refers to an antibody, an antibody fragment or other protein construct, such as a domain.

In one example, the present disclosure provides an expression system for an antigen binding molecule, wherein the antigen binding molecule is either secretable or membrane-bound, comprising:
a first antigen binding polynucleotide encoding a first part of the antigen binding molecule;
a cleavage polynucleotide encoding a cleavage site comprising a Furin consensus sequence RXKR (SEQ ID NO: 1) or RXRR (SEQ ID NO: 2), and a 2A polypeptide fragment thereof;
an anchor polynucleotide encoding a membrane anchor polypeptide;
   wherein the 2A polypeptide fragment thereof comprises one or more mutations in any one of the amino acid residue to control the cleavage efficiencies of the cleavage site to modulate ratio of production of the secretable antigen binding molecule versus the membrane-bound antigen binding molecule;
   wherein the cleavage polynucleotide is in between the first antigen binding polynucleotide and the anchor polynucleotide;
   wherein when the cleavage site is cleaved, the secretable antigen binding molecule comprising the first part of the antigen binding molecule is released;
   wherein when the cleavage site is not cleaved, the membrane-bound antigen binding molecule comprising the first part of the antigen binding molecule, the Furin consensus sequence RXKR (SEQ ID NO: 1) or RXRR (SEQ ID NO: 2), the 2A polypeptide fragment thereof, and the membrane anchor polypeptide, is released.

In another example, the present disclosure provides an expression system for an antigen binding molecule, wherein the antigen binding molecule is either secretable or membrane-bound, comprising:
a first antigen binding polynucleotide encoding a first part of the antigen binding molecule;
a first cleavage polynucleotide encoding a first cleavage site, wherein the first cleavage site is a minimal Furin cleavage consensus sequence RXKR (SEQ ID NO: 1) or RXRR (SEQ ID NO: 2);
a second cleavage polynucleotide encoding a self-processing second cleavage site, wherein the self-processing second cleavage site is a 2A polypeptide or a fragment thereof;
an anchor polynucleotide encoding a membrane anchor polypeptide;
   wherein the 2A polypeptide or the fragment thereof comprises one or more mutations in any one of the amino acid residue to control the cleavage efficiencies of the first cleavage site and the second cleavage site to modulate ratio of production of the secretable antigen binding molecule versus the membrane-bound antigen binding molecule;
   wherein the first and second cleavage polynucleotides are in between the first antigen binding polynucleotide and the anchor polynucleotide;
   wherein when the first cleavage site is cleaved, the secretable antigen binding molecule comprising the first part of the antigen binding molecule is released;
   wherein when the first and second cleavage sites are not cleaved, the membrane-bound antigen binding molecule comprising the first part of the antigen binding molecule, the minimal Furin cleavage consensus sequence RXKR (SEQ ID NO: 1) or RXRR (SEQ ID NO: 2), the 2A polypeptide or a fragment thereof, and the membrane anchor polypeptide, is released.

The term “polynucleotide” used herein refers to a nucleotide sequence that encodes for the product of interest, or a fragment, derivative, mutein, or variant thereof. A polynucleotide includes DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. The nomenclature of the polynucleotide is dependent on the product of interest that the polynucleotide it encodes. For example, a first or second antigen binding polynucleotide is the nucleotide sequence that encodes the first or second part of the antigen binding molecule as disclosed herein; a first or second cleavage polynucleotide is the nucleotide sequence that encodes the first or second cleavage site as disclosed herein; an anchor polynucleotide is the nucleotide sequence that encodes a membrane anchor polypeptide as disclosed herein. The same nomenclature would apply to any other polynucleotides as disclosed herein.

The first antigen binding polynucleotide can be at least 15 nucleotides in length. In another example, the first antigen binding polynucleotide can be, but is not limited to, a length of about 15 to 1500 nucleotides, or about 50, about 100, about 200, about 300, about 400, about 500, about 600, about 700, about 800, about 900, about 1000, about 1100, about 1200, about 1300, about 1400, or about 1500 nucleotides in length.

The first antigen binding polynucleotide encodes for a first part of the antigen binding molecule. In one example, the first part of the antigen binding molecule is an antibody or a fragment thereof. In another example, the first part of the antigen binding molecule is an antibody heavy chain. In another example, the first part of the antigen binding molecule is an antibody light chain.

The first antigen binding molecule can be at least 5 amino acids in length. In another example, the first antigen binding molecule can be, but is not limited to, a length of about 5 to 500 amino acids, or about 50, about 100, about 150, about 200, about 250, about 300, about 350, about 400, about 450, or about 500 amino acid residues in length.

The expression system of the present disclosure can further comprise a second antigen binding polynucleotide encoding a second part of the antigen binding molecule.

The second antigen binding polynucleotide can be at least 15 nucleotides in length. In another example, the second binding polynucleotide can be, but is not limited to, a length of about 15 to 700 nucleotides, or about 50, about 100, about 200, about 300, about 400, about 500, about 600, or about 700 nucleotides in length.

The second antigen binding polynucleotide encodes for a second part of the antigen binding molecule. In one example, the second part of the antigen binding molecule is an antibody or a fragment thereof. In another example, the second part of the antigen binding molecule is an antibody light chain. In another example, the second part of the antigen binding molecule is an antibody heavy chain.

The second antigen binding molecule can be at least 5 amino acids in length. In another example, the second antigen binding molecule can be, but is not limited to, a length of about 5 to 250 amino acids, or about 50, about 100, about 150, about 200 or about 250 amino acid residues in length.

Where the expression system encodes more than one cleavage site, the first cleavage polynucleotide of the expression system can be at least 9 nucleotides in length. In another example, the first cleavage polynucleotide can be, but is not limited to, a length of about 9 to 30 nucleotides, or about 10, about 15, about 20, about 25 or about 30 nucleotides in length. In a preferred example, the first cleavage polynucleotide is 12 nucleotides in length.

The first cleavage polynucleotide encodes for a first cleavage site. The first cleavage site can be at least 3 amino acids in length. In another example, the first cleavage site can be, but is not limited to, a length of about 3 to 10 amino acids, or about 3, 4, 5, 6, 7, 8, 9 or 10 amino acid residues in length. In a preferred example, the first cleavage site is 4 amino acids in length.

In one example, the first cleavage site is a minimal Furin cleavage consensus sequence. The term “Furin” used herein refers to a ubiquitous subtilisin-like proprotein convertase which cleaves proteins containing its recognition site. The terms not limiting to “Furin consensus sequence”, “minimal cleavage site”, “minimal Furin cleavage consensus sequence” or “Furin recognition site” as used herein refer to an amino acid sequence of RXKR (SEQ ID NO: 1) or RXRR (SEQ ID NO: 2), wherein X can be any amino acids. Furin recognises a protein that comprises a Furin consensus sequence, which leads to the cleavage of the protein that comprises the Furin consensus sequence. The cleavage can take place in, for example, the Golgi.

In one example, the Furin consensus sequence is selected from a group consisting of RRKR (SEQ ID NO: 3), RRRR (SEQ ID NO: 4), RSKR (SEQ ID NO: 5), RSRR (SEQ ID NO: 6), RKKR (SEQ ID NO: 7), RKRR (SEQ ID NO: 8), RQKR (SEQ ID NO: 9), RQRR (SEQ ID NO: 10), RTKR (SEQ ID NO: 11), RTRR (SEQ ID NO: 12), REKR (SEQ ID NO: 13), RERR (SEQ ID NO: 14), RDKR (SEQ ID NO: 15), RDRR (SEQ ID NO: 16), RHKR (SEQ ID NO: 17), RHRR (SEQ ID NO: 18), RFKR (SEQ ID NO: 19), RFRR (SEQ ID NO: 20), RAKR (SEQ ID NO: 21), RARR (SEQ ID NO: 22), RNKR (SEQ ID NO: 23), RNRR (SEQ ID NO: 24), RCKR (SEQ ID NO: RCRR (SEQ ID NO: 26), RGKR (SEQ ID NO: 27), RGRR (SEQ ID NO: 28), RIKR (SEQ ID NO: 29), RIRR (SEQ ID NO: 30), RLKR (SEQ ID NO: 31), RLRR (SEQ ID NO: 32), RMKR (SEQ ID NO: 33), RMRR (SEQ ID NO: 34), RPKR (SEQ ID NO: 35), RPRR (SEQ ID NO: 36), RWKR (SEQ ID NO: 37), RWRR (SEQ ID NO: 38), RYKR (SEQ ID NO: 39), RYRR (SEQ ID NO: 40), RVKR (SEQ ID NO: 41) and RVRR (SEQ ID NO: 42). In a preferred example, the Furin consensus sequence is RRKR (SEQ ID NO: 3).

The second cleavage polynucleotide of the expression system can be at least 10 nucleotides in length. In another example, the first cleavage polynucleotide can be, but is not limited to, a length of about 10 to 200 nucleotides, or about 10, about 20, about 30, about 40, about 50, about 60, about 70, about 80, about 90, about 100, about 110, about 120, about 130, about 140, about 150, about 160, about 170, about 180, about 190 or about 200 nucleotides in length. In a preferred example, the second cleavage polynucleotide is 15 nucleotides in length. In another preferred example, the second cleavage polynucleotide is 57 nucleotides in length.

The second cleavage polynucleotide encodes for a second cleavage site. The second cleavage site is a self-processing cleavage site. The term “self-processing cleavage site” as used herein refers to a peptide sequence that has self-cleaving or self-processing ability, wherein the peptide does not require an external molecule or enzyme such as a protease to cleave the peptide sequence. The second cleavage site can be at least 4 amino acids in length. In another example, the first cleavage site can be, but is not limited to, a length of about 4 to 50 amino acids, about 10 to 20 amino acids, about 20 to 30 amino acids, about 30 to 40 amino acids, about 40 to 50 amino acids, or about 4, 5, 6, 7, 8, 9, 10, 11, 12. 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 46, 47, 48, 49 or 50 amino acid residues in length. In one example, the first cleavage site is amino acids in length. In another example, the first cleavage site is 19 amino acids in length.

In one example, the self-processing second cleavage site is a 2A polypeptide or a fragment thereof. The terms not limiting to “2A polypeptide”, “2A peptide”, “2A protein” used herein refer to a peptide that mediates “self-cleavage” or “self-processing” of proteins during translation in eukaryotic cells. The 2A polypeptide is, for example, usually 18-25 amino-acid (aa) in length, and originates from viruses. 2A polypeptide is capable of self-cleaving, which occurs co-translationally between, for example, between the last two amino acids, glycine and proline. In one example, the 2A polypeptide or a fragment thereof is selected from a group consisting of P2A, F2A, E2A and T2A, or a fragment thereof. F2A is a 2A peptide derived from foot-and-mouth disease virus; E2A is a 2A peptide derived from equine rhinitis A virus; T2A is a 2A peptide derived from Thosea asigna virus; P2A is a 2A peptide derived from porcine teschovirus-1. In another example, the 2A polypeptide or a fragment thereof is a P2A polypeptide or a fragment thereof. In one example, the P2A polypeptide is ATNFSLLKQAGDVEENPGP (SEQ ID NO: 43). In another example, the F2A polypeptide is APVKQTLNFDLLKLAGDVESNPGP (SEQ ID NO: 44). In another example, the E2A polypeptide is QCTNYALLKLAGDVESNPGP (SEQ ID NO: 45). In another example, the T2A polypeptide is EGRGSLLTCGDVEENPGP (SEQ ID NO: 46). The corresponding nucleotide sequences are shown in Table 1 below.

TABLE 1
Nucleotide and amino acid sequences of P2A, F2A, E2A and T2A.

| Item | Nucleotide sequence | Amino acid sequence |
|---|---|---|
| P2A | GCT ACT AAC TTC AGC CTG CTG AAG | ATNFSLLKQAGDVEENPGP |
| | CAG GCT GGA GAC GTG GAG GAG | (SEQ ID NO: 43) |
| | AAC CCT GGG CCC | |
| | (SEQ ID NO: 47) | |
| F2A | GCA CCG GTG AAA CAG ACT TTG AAT | APVKQTLNFDLLKLAGDVESNPG |
| | TTT GAC CTT CTG AAG TTG GCA GGA | P |
| | GAC GTT GAG TCC AAC CCT GGG CCC | (SEQ ID NO: 44) |
| | (SEQ ID NO: 48) | |
| E2A | CAG TGT ACT AAT TAT GCT CTC TTG | QCTNYALLKLAGDVESNPGP |
| | AAA TTG GCT GGA GAT GTT GAG AGC | (SEQ ID NO: 45) |
| | AAC CCT GGG CCC | |
| | (SEQ ID NO: 49) | |
| T2A | GAG GGC AGA GGA AGT CTG CTA | EGRGSLLTCGDVEENPGP |
| | ACA TGC GGT GAC GTC GAG GAG | (SEQ ID NO: 46) |
| | AAT CCT GGG CCC | |
| | (SEQ ID NO: 50) | |

Where the expression system encodes one cleavage site, the expression system comprises a cleavage polynucleotide encoding a cleavage site comprising a Furin consensus sequence as disclosed herein, and a 2A polypeptide fragment thereof. It would also be understood that the 2A polypeptide fragment thereof refers to a section of the 2A polypeptide as disclosed herein. In one example, the 2A polypeptide fragment thereof comprises a section of at least 3 amino acids from the 2A polypeptide. In another example, the 2A polypeptide fragment thereof comprises a section of, but is not limited to, about 3-10 amino acids, or about 3, 4, 5, 6, 7, 8, 9, or 10 amino acids from the 2A polypeptide. In another example, the 2A polypeptide fragment thereof is selected from a group consisting of P2A, F2A, E2A and T2A fragment thereof. In another example, the 2A polypeptide fragment thereof is a P2A polypeptide fragment thereof.

In one example, the 2A polypeptide fragment thereof can be the first 3-10 amino acids of the 2A polypeptide as disclosed herein, or the last 3-10 amino acids of the 2A polypeptide as disclosed herein. In another example, the 2A polypeptide fragment thereof is the first 5 amino acids of a P2A, F2A, E2A or T2A polypeptide. In another example, the P2A polypeptide fragment thereof is the first 5 amino acids of a P2A polypeptide. In another example, the 2A polypeptide fragment thereof is ATNFS (SEQ ID NO: 51).

The 2A polypeptide or the 2A polypeptide fragment thereof encoded by the expression system comprises one or more mutations. An amino acid mutation to, for example, proline or glycine in a 2A polypeptide or the 2A polypeptide fragment thereof can influence the secondary structure of the 2A polypeptide or the 2A polypeptide fragment thereof, by constraining or providing high flexibility to peptide chains, respectively.

In expression system that encodes more than one cleavage site, such mutations can control the cleavage efficiencies of the first cleavage site and the second cleavage site to modulate ratio of production of the secretable antigen binding molecule versus the membrane-bound antigen binding molecule. In one example, the 2A polypeptide or a fragment thereof comprises one or more mutations in amino acid residue 1, 2, 3, 4 or 5. It would be understood that the amino acid residue 1, 2, 3, 4 or 5 refers to the amino acid residue 1, 2, 3, 4 or 5 of the 2A polypeptide or fragment thereof. In one example, amino acid residue 1, 2, 3, 4 or 5 of the 2A polypeptide or the fragment thereof is mutated to any one selected from the group consisting of glycine, proline, alanine, arginine, histidine, lysine, aspartic acid, glutamic acid, serine, threonine, asparagine, glutamine, cysteine, selenocysteine, valine, isoleucine, leucine, methionine, phenylalanine, tyrosine and tryptophan. In another example, amino acid residue 1, 2, 3, 4 or 5 of the 2A polypeptide or fragment thereof is mutated to glycine, proline or alanine.

In one example, the one or more mutations in 2A polypeptide or a fragment thereof is selected from the group consisting of A1P, A1G, T2G, T2P, N3P, F4P, S5P, N3A and F4A. If the mutations occur in the P2A polypeptide, the mutations result in the following P2A polypeptide sequences: PTNFSLLKQAGDVEENPGP (SEQ ID NO: 109), GTNFSLLKQAGDVEENPGP (SEQ ID NO: 101), AGNFSLLKQAGDVEENPGP (SEQ ID NO: 108), APNFSLLKQAGDVEENPGP (SEQ ID NO: 107), ATPFSLLKQAGDVEENPGP (SEQ ID NO: 103), ATNPSLLKQAGDVEENPGP (SEQ ID NO: 105), ATNFPLLKQAGDVEENPGP (SEQ ID NO: 106), ATAFSLLKQAGDVEENPGP (SEQ ID NO: 104) and ATNASLLKQAGDVEENPGP (SEQ ID NO: 102) respectively. In another example, the mutation in 2A polypeptide or a fragment thereof comprises A1P, A1G, T2G or T2P. In another example, the mutation in 2A polypeptide or a fragment thereof is A1P. In another example, the mutation in 2A polypeptide or a fragment thereof is A1G. In another example, the mutation in 2A polypeptide or a fragment thereof is T2G. In another example, the mutation in 2A polypeptide or a fragment thereof is T2P.

In the expression system that encodes one cleavage site, such mutations can control the cleavage efficiencies of the cleavage site to modulate ratio of production of the secretable antigen binding molecule versus the membrane-bound antigen binding molecule. In another example, the 2A polypeptide fragment thereof comprises one or more mutations in amino acid residue 1, 2, 3, 4 or 5. It would be understood that the amino acid residue 1, 2, 3, 4 or 5 refers to the amino acid residue 1, 2, 3, 4 or 5 of the 2A polypeptide fragment thereof. In one example, amino acid residue 1, 2, 3, 4 or 5 of the 2A polypeptide fragment thereof is mutated to any one selected from the group consisting of glycine, proline, alanine, arginine, histidine, lysine, aspartic acid, glutamic acid, serine, threonine, asparagine, glutamine, cysteine, selenocysteine, valine, isoleucine, leucine, methionine, phenylalanine, tyrosine and tryptophan. In another example, amino acid residue 1, 2, 3, 4 or 5 of the 2A polypeptide fragment thereof is mutated to glycine, proline or alanine. In another example, the one or more mutations in 2A polypeptide fragment thereof is selected from the group consisting of A1P, A1G, T2G, T2P, N3P, F4P, S5P, N3A and F4A. The mutations result in the following 2A polypeptide fragment sequences: PTNFS (SEQ ID NO: 60), GTNFS (SEQ ID NO: 52), AGNFS (SEQ ID NO: 59), APNFS (SEQ ID NO: 58), ATPFS (SEQ ID NO: 54), ATNPS (SEQ ID NO: 56), ATNFP (SEQ ID NO: 57), ATAFS (SEQ ID NO: 55) and ATNAS (SEQ ID NO: 53) respectively. In another example, the mutation in 2A polypeptide fragment thereof is A1P. In another example, the mutation in 2A polypeptide fragment thereof is A1G. In another example, the mutation in 2A polypeptide fragment thereof is T2G. In another example, the mutation in 2A polypeptide fragment thereof is T2P.

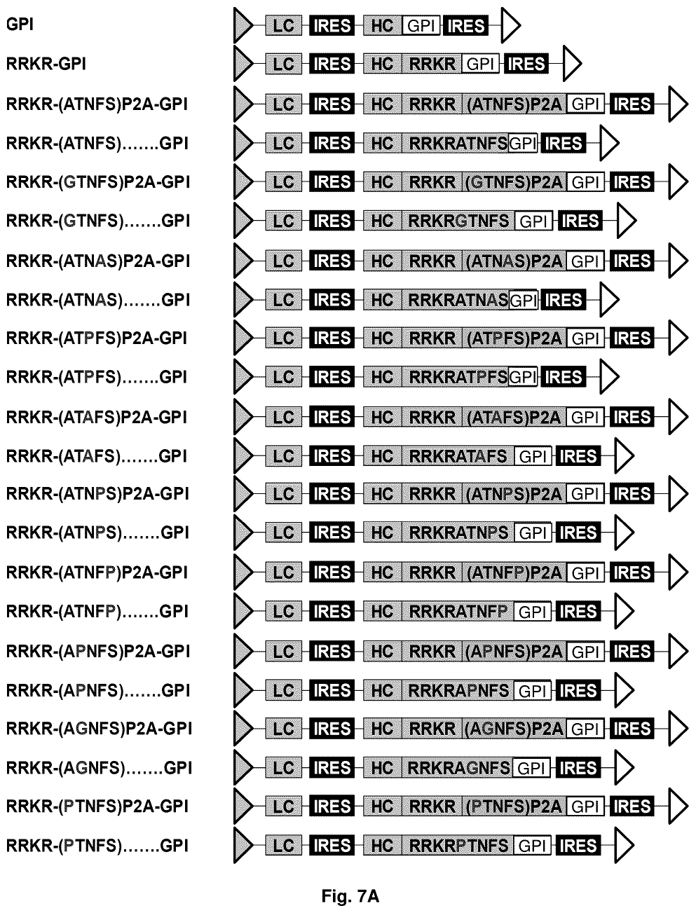

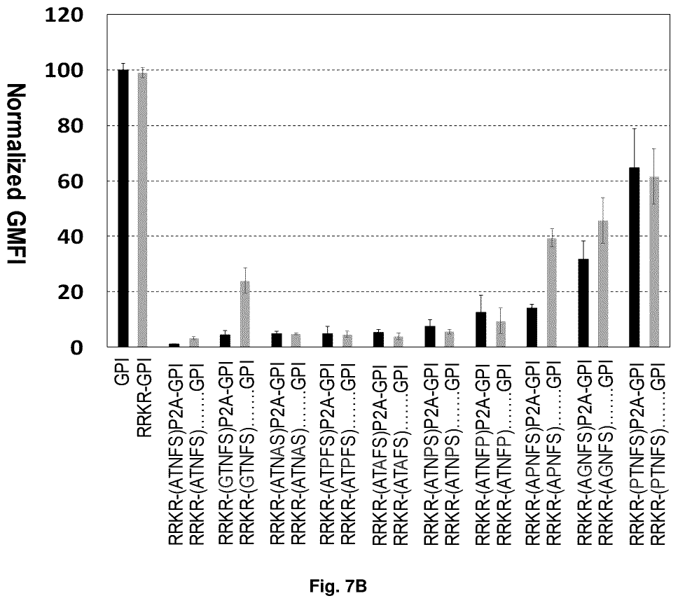

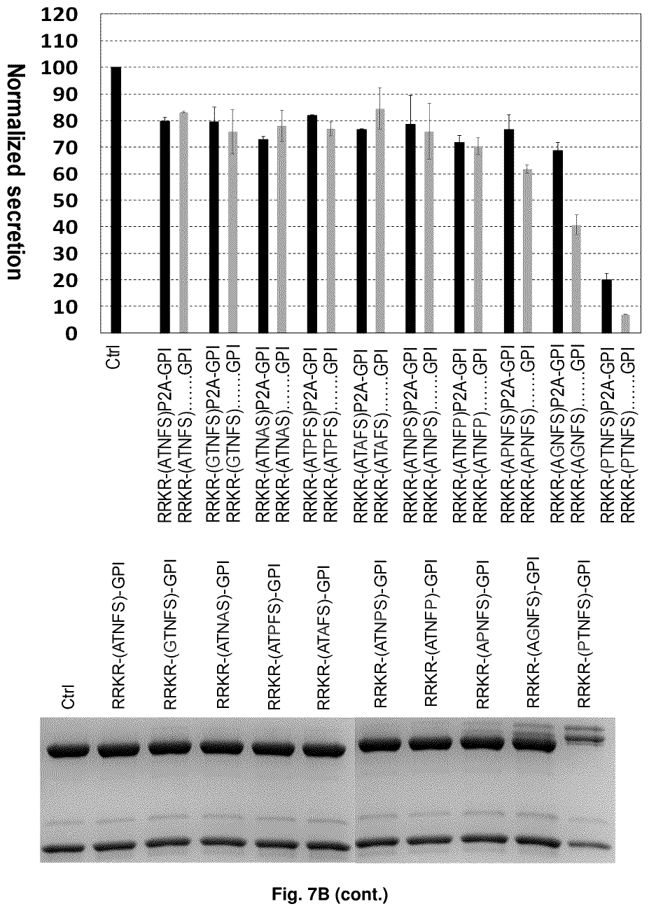

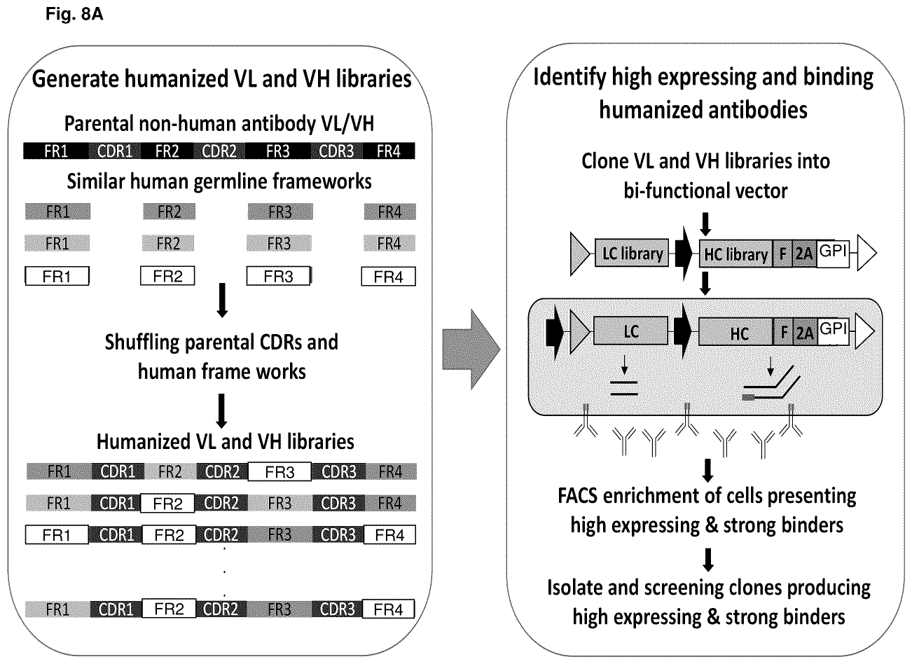

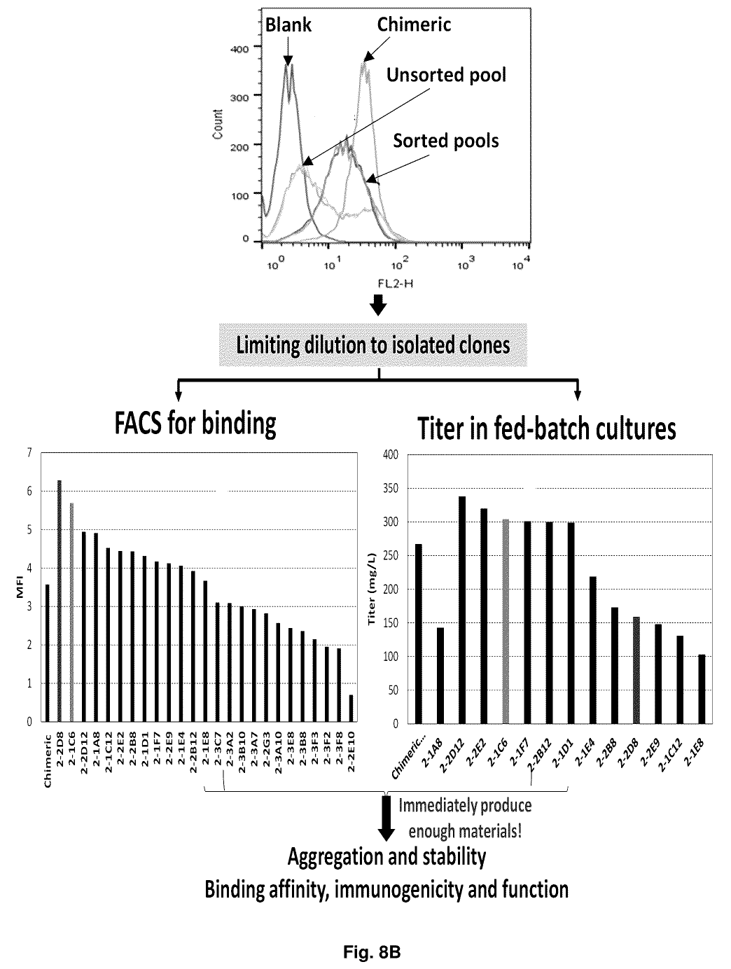

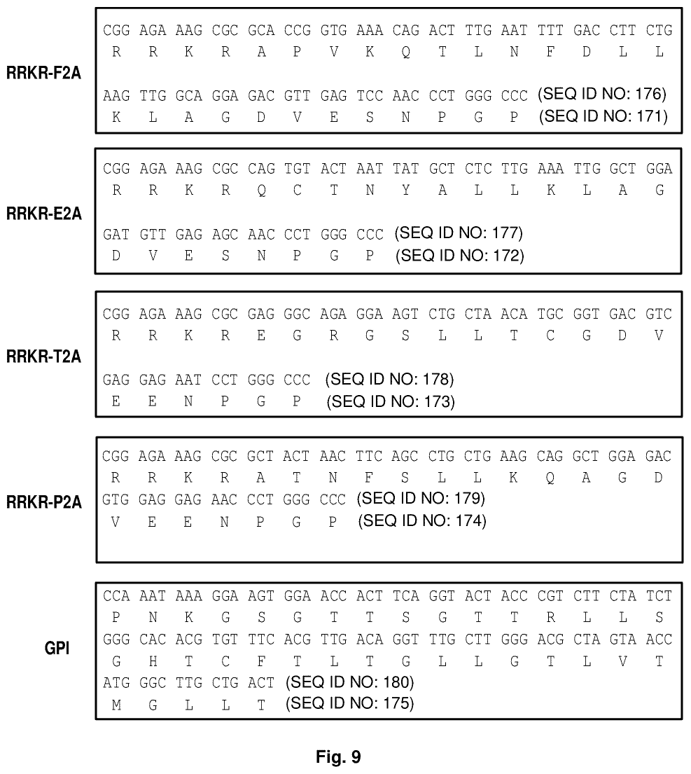

The expression system can comprise a first and second cleavage polynucleotide encoding a first cleavage site comprising a Furin consensus sequence and a second cleavage site comprising a 2A polypeptide or fragment thereof respectively, and can include different combinations of the Furin consensus sequence and 2A polypeptide or fragment thereof as disclosed herein. In one example, the first and second cleavage sites comprises a sequence selected from a group consisting of RXKRPTNFSLLKQAGDVEENPGP (SEQ ID NO: 139), RXKRGTNFSLLKQAGDVEENPGP (SEQ ID NO: 131), RXKRAGNFSLLKQAGDVEENPGP (SEQ ID NO: 138), RXKRAPNFSLLKQAGDVEENPGP (SEQ ID NO: 137), RXKRATPFSLLKQAGDVEENPGP (SEQ ID NO: 133), RXKRATNPSLLKQAGDVEENPGP (SEQ ID NO: 135), RXKRATNFPLLKQAGDVEENPGP (SEQ ID NO: 136), RXKRATAFSLLKQAGDVEENPGP (SEQ ID NO: 134), RXKRATNASLLKQAGDVEENPGP (SEQ ID NO: 132), RXKRATNFSLLKQAGDVEENPGP (SEQ ID NO: 130), RXRRATNFSLLKQAGDVEENPGP (SEQ ID NO: 140), RXRRGTNFSLLKQAGDVEENPGP (SEQ ID NO: 141), RXRRATNASLLKQAGDVEENPGP (SEQ ID NO: 142), RXRRATPFSLLKQAGDVEENPGP (SEQ ID NO: 143), RXRRATAFSLLKQAGDVEENPGP (SEQ ID NO: 144), RXRRATNPSLLKQAGDVEENPGP (SEQ ID NO: 145), RXRRATNFPLLKQAGDVEENPGP (SEQ ID NO: 146), RXRRAPNFSLLKQAGDVEENPGP (SEQ ID NO: 147), RXRRAGNFSLLKQAGDVEENPGP (SEQ ID NO: 148), and RXRRPTNFSLLKQAGDVEENPGP (SEQ ID NO: 149). In another example, the first and second cleavage sites comprises a sequence selected from a group consisting of RRKRPTNFSLLKQAGDVEENPGP (SEQ ID NO: 119), RRKRGTNFSLLKQAGDVEENPGP (SEQ ID NO: 111), RRKRAGNFSLLKQAGDVEENPGP (SEQ ID NO: 118), RRKRAPNFSLLKQAGDVEENPGP (SEQ ID NO: 117), RRKRATPFSLLKQAGDVEENPGP (SEQ ID NO: 113), RRKRATNPSLLKQAGDVEENPGP (SEQ ID NO: 115), RRKRATNFPLLKQAGDVEENPGP (SEQ ID NO: 116), RRKRATAFSLLKQAGDVEENPGP (SEQ ID NO: 114), RRKRATNASLLKQAGDVEENPGP (SEQ ID NO: 112), RRKRATNFSLLKQAGDVEENPGP (SEQ ID NO: 110), RRRRATNFSLLKQAGDVEENPGP (SEQ ID NO: 120), RRRRGTNFSLLKQAGDVEENPGP (SEQ ID NO: 121), RRRRATNASLLKQAGDVEENPGP (SEQ ID NO: 122), RRRRATPFSLLKQAGDVEENPGP (SEQ ID NO: 123), RRRRATAFSLLKQAGDVEENPGP (SEQ ID NO: 124), RRRRATNPSLLKQAGDVEENPGP (SEQ ID NO: 125), RRRRATNFPLLKQAGDVEENPGP (SEQ ID NO: 126), RRRRAPNFSLLKQAGDVEENPGP (SEQ ID NO: 127), RRRRAGNFSLLKQAGDVEENPGP (SEQ ID NO: 128), and RRRRPTNFSLLKQAGDVEENPGP (SEQ ID NO: 129). Examples of such cleavage sites can be found in the expression system shown in FIG. 7A.

The expression system can comprise a cleavage polynucleotide encoding a cleavage site comprising a Furin consensus sequence and a 2A polypeptide fragment thereof can include different combinations of the Furin consensus sequence and a 2A polypeptide fragment thereof as disclosed herein. In one example, the cleavage site comprises a sequence selected from a group consisting of RXKRPTNFS (SEQ ID NO: 90), RXKRGTNFS (SEQ ID NO: 82), RXKRAGNFS (SEQ ID NO: 89), RXKRAPNFS (SEQ ID NO: 88), RXKRATPFS (SEQ ID NO: 84), RXKRATNPS (SEQ ID NO: 86), RXKRATNFP (SEQ ID NO: 87), RXKRATAFS (SEQ ID NO: 85), RXKRATNAS (SEQ ID NO: 83), RXKRATNFS (SEQ ID NO: 81), RXRRATNFS (SEQ ID NO: 91), RXRRGTNFS (SEQ ID NO: 92), RXRRATNAS (SEQ ID NO: 93), RXRRATPFS (SEQ ID NO: 94), RXRRATAFS (SEQ ID NO: 95), RXRRATNPS (SEQ ID NO: 96), RXRRATNFP (SEQ ID NO: 97), RXRRAPNFS (SEQ ID NO: 98), RXRRAGNFS (SEQ ID NO: 99), and RXRRPTNFS (SEQ ID NO: 100). In another example, the cleavage site comprises a sequence selected from a group consisting of RRKRPTNFS (SEQ ID NO: 70), RRKRGTNFS (SEQ ID NO: 62), RRKRAGNFS (SEQ ID NO: 69), RRKRAPNFS (SEQ ID NO: 68), RRKRATPFS (SEQ ID NO: 64), RRKRATNPS (SEQ ID NO: 66), RRKRATNFP (SEQ ID NO: 67), RRKRATAFS (SEQ ID NO: 65), RRKRATNAS (SEQ ID NO: 63), RRKRATNFS (SEQ ID NO: 61), RRRRATNFS (SEQ ID NO: 71), RRRRGTNFS (SEQ ID NO: 72), RRRRATNAS (SEQ ID NO: 73), RRRRATPFS (SEQ ID NO: 74), RRRRATAFS (SEQ ID NO: 75), RRRRATNPS (SEQ ID NO: 76), RRRRATNFP (SEQ ID NO: 77), RRRRAPNFS (SEQ ID NO: 78), RRRRAGNFS (SEQ ID NO: 79), and RRRRPTNFS (SEQ ID NO: 80). In another example, the cleavage site sequence is RRKRPTNFS (SEQ ID NO: 70). In another example, the cleavage site sequence is RRKRGTNFS (SEQ ID NO: 62). In another example, the cleavage site sequence is RRKRAGNFS (SEQ ID NO: 69). In another example, the cleavage site sequence is RRKRAPNFS (SEQ ID NO: 68). In one example, the cleavage site sequence is RRKRATNFS (SEQ ID NO: 61). In another example, the cleavage site sequence is RRKRATNAS (SEQ ID NO: 63). In another example, the cleavage site sequence is RRKRATPFS (SEQ ID NO: 64). In another example, the cleavage site sequence is RRKRATAFS (SEQ ID NO: 65). In another example, the cleavage site sequence is RRKRATNPS (SEQ ID NO: 66). In another example, the cleavage site sequence is RRKRATNFP (SEQ ID NO: 67). Examples of such cleavage sites can be found in the expression system shown in FIG. 7A.

The anchor polynucleotide of the expression system can be at least 20 nucleotides in length. In another example, the first cleavage polynucleotide can be, but is not limited to, a length of about 20 to 130 nucleotides, or about 10, about 20, about 30, about 40, about 50, about 60, about 70, about 80, about 90, about 100, about 110, about 120 or about 130 nucleotides in length. In a preferred example, the first cleavage polynucleotide is 111 nucleotides in length.

The anchor polynucleotide encodes for a membrane anchor polypeptide. The membrane anchor polypeptide can be at least 7 amino acids in length. In another example, the membrane anchor polypeptide can be, but is not limited to, a length of about 7 to 45 amino acids, about 15 to 40 amino acids, or about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 amino acid residues in length. In a preferred example, the membrane anchor polypeptide is 37 amino acids in length.

In one example, the membrane anchor polypeptide comprises glycophospholipid transmembrane domain (GPI), platelet-derived growth factor receptor (PDGFR) beta chain transmembrane domain (PTM), or immunoglobulin C2-type extracellular-transmembrane-cytosolic domains of murin B7-1 antigen. In another example, the membrane anchor polypeptide is glycophospholipid transmembrane domain (GPI). The terms not limiting to “glycophospholipid transmembrane domain”, “glycophospholipid membrane anchor”, “GM” or “GPI” used herein refer to membrane proteins that are anchored to a membrane in a cell by a structure comprising phosphatidylinositol, carbohydrate, and ethanolamine. In one example, the glycophospholipid transmembrane domain (GPI) is a glycosidylphosphatidylinositol membrane anchor derived from human decay-accelerating factor. In one example, the amino acid sequence of glycophospholipid transmembrane domain (GPI) is SEQ ID NO: 175.

One purpose of the expression system of the present disclosure is to screen for the expression levels of the secretable and/or membrane-bound antigen binding molecule, otherwise known as dual screening. To modulate ratio of production of the secretable antigen binding molecule versus the membrane-bound antigen binding molecule, cleavage efficiencies of the cleavage sites, for example, the first cleavage site and the second cleavage site, can be controlled by, for example, the type of cleavage sites and/or the mutations present in the cleavage site. Depending on the efficiencies of the cleavage sites, complete cleavage or incomplete cleavage can occur to the proteins that are produced by the expression system of the present disclosure.

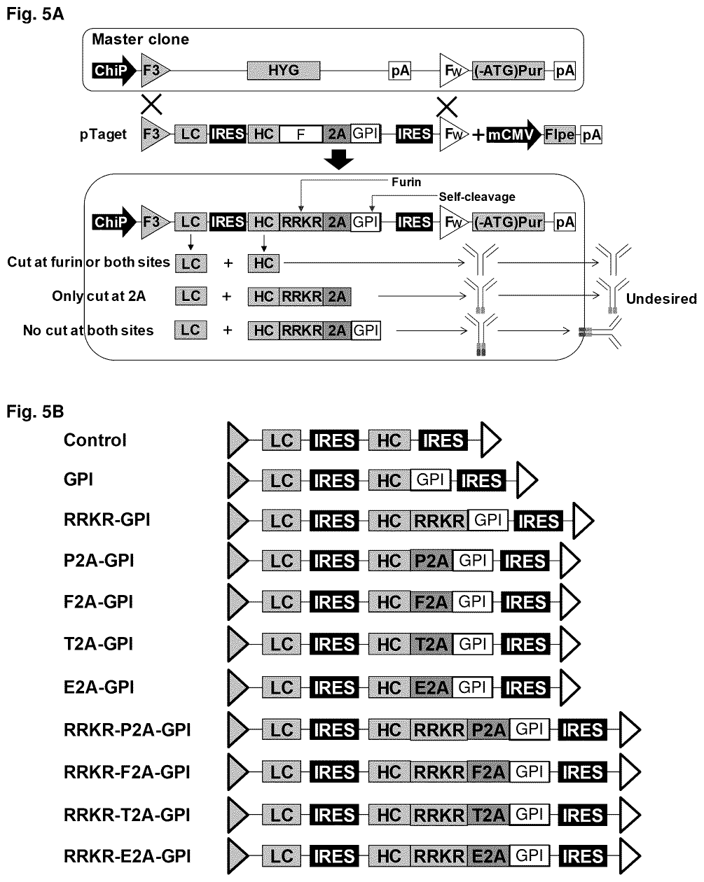

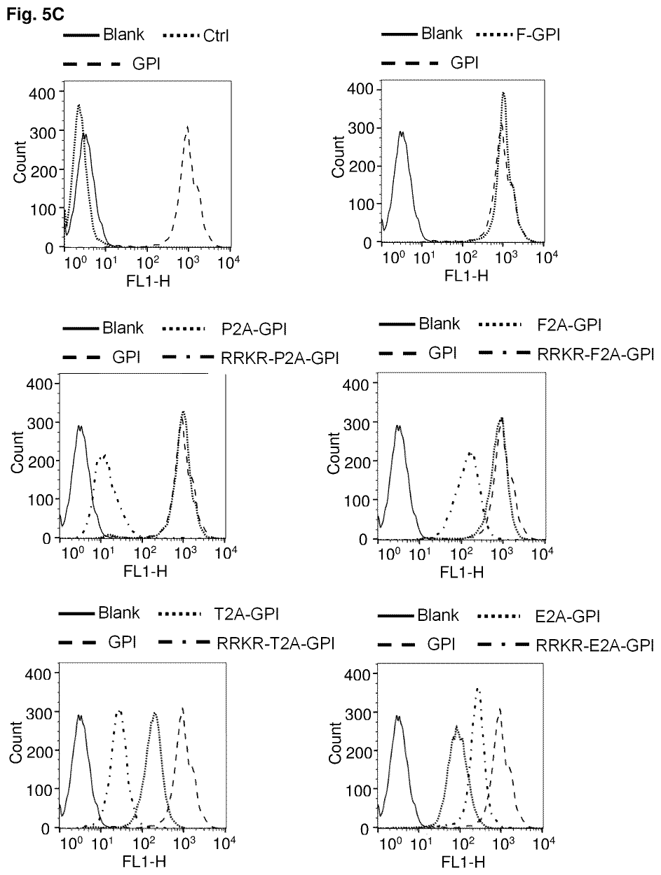

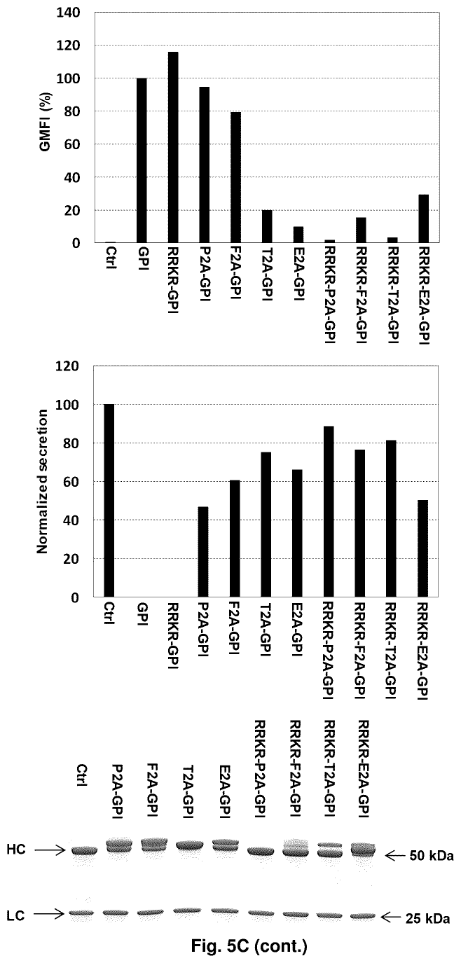

The terms not limiting to “incomplete cleavage” or “partial cleavage” used herein refer to antigen binding molecules in one cell that are differentially cleaved at the Furin and/or 2A cleavage sites, resulting in a mixture of: antigen binding molecule that is cleaved only at the Furin cleavage site resulting in a secretable antigen binding molecule; antigen binding molecule that is cleaved only at the 2A cleavage site resulting in an incorrect secretable antigen binding molecule; antigen binding molecule that is cleaved at both Furin and 2A cleavage sites; and antigen binding molecule that are not cleaved at both Furin and 2A cleavage sites resulting in a membrane bound antigen binding molecule, as shown in the schematic presented in FIG. 5A. In a preferred embodiment, incomplete cleavage occurs when antigen binding molecules are differentially cleaved at the RRKR (SEQ ID NO: 3) and/or P2A cleavage sites. Different methods can be used to quantify the levels of incomplete cleavage. In one example, the ratio of secreted antigen binding molecule (concentration of antibodies in the culture supernatant) to the membrane bound antigen binding molecule can be used to quantify the levels of incomplete cleavage. In a preferred example, the ratio of secreted antibody (concentration of antibodies in the culture supernatant) to the membrane bound antibody (measured by staining and fluorescence-activated cell sorting (FACS)) indirectly indicates the relative ratio of the molecules with partial cleavage at RRKR (SEQ ID NO: 3) and/or 2A to the molecules without cleavage at both sites expressed in a cell.

The term “complete cleavage” as used herein refers to all antigen binding molecules in one cell that are cleaved at both the Furin cleavage site and the 2A cleavage site, resulting in a secretable antigen binding molecule, as shown in the schematic presented in FIG. 5A. In a preferred embodiment, complete cleavage occurs when cleavage happens at the RRKR (SEQ ID NO: 3) and P2A cleavage sites.

The term “membrane bound antigen binding molecule” as used herein refers to an antigen binding molecule comprising a first and second part of the antigen binding molecule, a Furin consensus sequence, a 2A polypeptide or a fragment thereof and a membrane anchor polypeptide derived from the expression system as disclosed herein that is produced and released from a cell. In a one example, the membrane bound antigen binding molecule comprises a light and heavy chain of an antibody, RXKR (SEQ ID NO: 1) or RXRR (SEQ ID NO: 2) sequence, a P2A polypeptide or a fragment thereof and a membrane anchor polypeptide. In a preferred example, the membrane bound antigen binding molecule comprises a light and heavy chain of an antibody, RRKR (SEQ ID NO: 3) sequence, a P2A polypeptide or a fragment thereof and a membrane anchor polypeptide.

The levels of membrane bound antigen binding molecule is determined by the display level. The term “display level” used herein refers to the expression of a membrane-bound antigen binding molecule on a cell surface, wherein the membrane-bound antigen binding molecule comprises: a first antigen binding polynucleotide encoding a first part of the antigen binding molecule; a second antigen binding polynucleotide encoding a second part of the antigen binding molecule.

The terms “secretable antigen binding molecule”, “correct product” or “desired product” used herein refer to an antigen binding molecule comprising a first and second part of the antigen binding molecule derived from the expression system as disclosed herein that is produced and released from a cell. In a preferred example, the secretable antigen binding molecule comprises a light and heavy chain of an antibody.

The terms not limiting to “incorrect secretable antigen binding molecule”, “incorrect product” or “undesired product” used herein refer to an antigen binding molecule comprising a first and second part of the antigen binding molecule, a Furin consensus sequence and a 2A polypeptide or a fragment thereof derived from the expression system as disclosed herein that is produced and released from a cell. In a preferred example, the incorrect secretable antigen binding molecule comprises a light and heavy chain of an antibody, RRKR (SEQ ID NO: 3) sequence and a P2A polypeptide or a fragment thereof.

The expression system of the present disclosure can further comprise one or more internal ribosome entry site (IRES) polynucleotide. In one example, the one or more IRES polynucleotide is before the first antigen binding polynucleotide. In another example, the one or more IRES polynucleotide is after the anchor polynucleotide. In one example, the nucleotide sequence of the one or more IRES polynucleotide is SEQ ID NO: 181. In one example, the one or more IRES polynucleotide encodes for a wild-type encephalomyocarditis virus (EMCV) internal ribosome entry site (IRES).

The expression system of the present disclosure can also further comprise one or more operably linked promoter sequences. The term “operably linked” refers to a functional linkage between a transcription regulating nucleotide sequence (for example, a promoter sequence) and other nucleotide sequences. Thus, the transcription regulating nucleotide sequence may regulate transcription and/or translation of other nucleotide sequences. In one example, the one or more operably linked promoter sequence is selected from a group consisting of ChiP, human CMV, murine CMV, SV40, human EF promoters. In a preferred example, the promoter sequence is ChiP. ChiP is a chimeric promoter consisting of murine cytomegalovirus (CMV) enhancer, human CMV core promoter and human CMV intron A. In one example, the nucleotide sequence of the one or more operably linked promoter sequences is SEQ ID NO: 182.

Different combinations of expressions systems can be generated. In one example, the expression system comprises: a second antigen binding polynucleotide encoding a light chain antibody; a first antigen binding polynucleotide encoding a heavy chain antibody; a first cleavage polynucleotide encoding a Furin consensus sequence RXKR (SEQ ID NO: 1) or RXRR (SEQ ID NO: 2); a second cleavage polynucleotide encoding a 2A polypeptide or a fragment thereof; an anchor polynucleotide encoding a membrane anchor polypeptide; wherein the 2A polypeptide or a fragment thereof comprises one or more mutations. In another example, the expression system comprises a polynucleotide encoding an operably linked promoter sequence; a second antigen binding polynucleotide encoding a light chain antibody; a first IRES polynucleotide; a first antigen binding polynucleotide encoding a heavy chain antibody; a first cleavage polynucleotide encoding a Furin consensus sequence RXKR (SEQ ID NO: 1) or RXRR (SEQ ID NO: 2); a second cleavage polynucleotide encoding a 2A polypeptide or a fragment thereof; an anchor polynucleotide encoding a membrane anchor polypeptide; a second IRES polynucleotide; wherein the 2A polypeptide or a fragment thereof comprises one or more mutations.

In another example, the expression system comprises: a second antigen binding polynucleotide encoding a light chain antibody; a first antigen binding polynucleotide encoding a heavy chain antibody; a cleavage polynucleotide encoding a Furin consensus sequence RXKR (SEQ ID NO: 1) or RXRR (SEQ ID NO: 2) and a 2A polypeptide fragment thereof; an anchor polynucleotide encoding a membrane anchor polypeptide; wherein the 2A polypeptide fragment thereof comprises one or more mutations. In another example, the expression system comprises: a second antigen binding polynucleotide encoding a light chain antibody; a first antigen binding polynucleotide encoding a heavy chain antibody; a cleavage polynucleotide encoding RRKRGTNFS (SEQ ID NO: 62); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRAPNFS (SEQ ID NO: 68); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRATNAS (SEQ ID NO: 63); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRATPFS (SEQ ID NO: 64); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRATAFS (SEQ ID NO: 65); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRATNPS (SEQ ID NO: 66); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRATNFP (SEQ ID NO: 67); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRAGNFS (SEQ ID NO: 69); an anchor polynucleotide encoding a membrane anchor polypeptide. In another example, the expression system comprises: a second antigen binding polynucleotide encoding a light chain antibody; a first antigen binding polynucleotide encoding a heavy chain antibody; a cleavage polynucleotide encoding RRKRPTNFS (SEQ ID NO: 70); an anchor polynucleotide encoding a membrane anchor polypeptide.

In yet another example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding a Furin consensus sequence RXKR (SEQ ID NO: 1) or RXRR (SEQ ID NO: 2) and a 2A polypeptide fragment thereof; an anchor polynucleotide encoding a membrane anchor polypeptide; a second IRES polynucleotide; wherein the 2A polypeptide fragment thereof comprises one or more mutations. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRGTNFS (SEQ ID NO: 62); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRAPNFS (SEQ ID NO: 68); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRATNAS (SEQ ID NO: 63); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRATPFS (SEQ ID NO: 64); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRATAFS (SEQ ID NO: 65); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRATNPS (SEQ ID NO: 66); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRATNFP (SEQ ID NO: 67); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRAGNFS (SEQ ID NO: 69); an anchor polynucleotide encoding a membrane anchor polypeptide; a second IRES polynucleotide. In one example, the expression system comprises: a polynucleotide encoding an operably linked promoter sequence; a second antigen binding polynucleotide encoding a light chain antibody; a first IRES polynucleotide; a first antigen binding polynucleotide encoding a heavy chain antibody; a cleavage polynucleotide encoding RRKRPTNFS (SEQ ID NO: 70); an anchor polynucleotide encoding a membrane anchor polypeptide; a second IRES polynucleotide.

In one example, the expression system as disclosed herein comprises any one of the sequences selected from the group consisting of SEQ ID NOs: 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187. 188, 189, 190, 191, 192, 193, 194, 195, 196, 197, 198, 199, 200, 201, 202, 203, 204, 205 and 206. In one example, the expression system comprises SEQ ID NO: 170. In another example, the expression system comprises SEQ ID NO: 154. In another example, the expression system comprises SEQ ID NO: 168. In another example, the expression system comprises SEQ ID NO: 166. In another example, the expression system comprises SEQ ID NO: 169. In another example, the expression system comprises SEQ ID NO: 153. In another example, the expression system comprises SEQ ID NO: 167. In another example, the expression system comprises SEQ ID NO: 165.

There is provided a vector comprising the expression system as disclosed herein. The term “vector” as used herein refers to a means, typically a nucleic acid, of transporting and expressing a target gene in a host cell. For example, the vector may include a plasmid vector, a cosmid vector, or a virus vector, such as a bacteriophage vector, an adenovirus vector, a retrovirus vector, and an adeno-associated virus vector. The recombinant vector may be prepared by manipulating a plasmid, a phage, or a virus known in the art.

There is also provided a host cell comprising the expression system or vector as disclosed herein. The host cell may be prokaryotic or eukaryotic host cells. The host cell, which is capable of stably and continuously cloning or expressing the expression system or vector, may be any host cell known in the art.

There is also provided a kit comprising the expression system, vector or host cell as disclosed herein. The kit can further comprise, but is not limited to, buffer or cell culture media known in the art.

The expression system as disclosed herein can be used in a method for detecting the presence of secreted antibodies and/or surface-bound antibodies in a sample. In one example, the method for detecting the presence of one or more secreted antibodies and/or one or more surface-bound antibodies in a sample comprises: providing an expression system as disclosed herein; delivering said expression system to one or more target cells, wherein said target cell transcribes said expression system, wherein once transcribed, said first cleavage site is cleaved in a first plurality of first part of the antigen binding molecule, so that said first plurality of first part of the antigen binding molecule do not comprise said membrane anchor polypeptide, and are thereby secreted by said target cell, and wherein said first and second cleavage sites are not cleaved in a second plurality of first part of the antigen binding molecule, so that said second plurality of first part of the antigen binding molecule comprise said membrane anchor polypeptide, and are thereby bound to the surface of said target cell; and detecting the presence or absence of said first plurality of first part of the antigen binding molecule secreted by said target cell and/or detecting a quantity of said second plurality of first part of the antigen binding molecule on the surface of said target cell.

In another example, the method for detecting the presence of one or more secreted antibodies and/or one or more surface-bound antibodies in a sample comprises: providing an expression system as disclosed herein; delivering said expression system to one or more target cells, wherein said target cell transcribes said expression system, wherein once transcribed, said cleavage site is cleaved in a first plurality of first part of the antigen binding molecule, so that said first plurality of first part of the antigen binding molecule do not comprise said membrane anchor polypeptide, and are thereby secreted by said target cell, and wherein said cleavage sites are not cleaved in a second plurality of first part of the antigen binding molecule, so that said second plurality of first part of the antigen binding molecule comprise said membrane anchor polypeptide, and are thereby bound to the surface of said target cell; and detecting the presence or absence of said first plurality of first part of the antigen binding molecule secreted by said target cell and/or detecting a quantity of said second plurality of first part of the antigen binding molecule on the surface of said target cell.

The expression system, vector, host cell or kit as disclosed herein thus has different applications. In one example, the expression system, vector, host cell or kit as disclosed herein is for use in antibody discovery. In another example, the expression system, vector, host cell or kit as disclosed herein is for use in screening antibody libraries. In another example, the expression system, vector, host cell or kit as disclosed herein is for use in antibody humanization. In another example, the expression system, vector, host cell or kit as disclosed herein is for use in affinity maturation. In another example, the expression system. vector, host cell or kit as disclosed herein is for use in antibody production, including but not limited to monoclonal antibodies or polyclonal antibodies.

As used in this application, the singular form “a,” “an,” and “the” include plural references unless the context clearly dictates otherwise. For example, the term “a genetic marker” includes a plurality of genetic markers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Material and Methods

### Generation of CHO Master Clone for Single Gene Integration

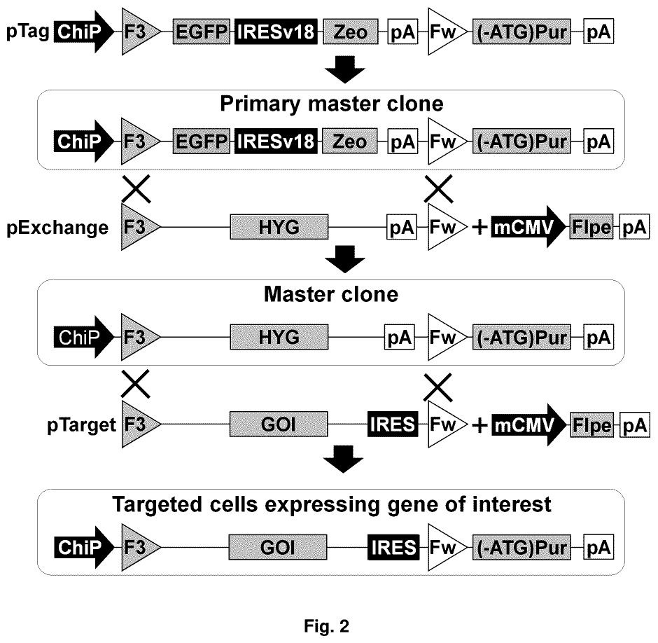

A master clone for display and secretion of antibodies needs to have a single integration site which is able to provide stable and high antibody expression and allows efficient targeted integration of antibody genes. The process of generating such a master clone is summarized in FIG. 2. Adherent CHO K1 cells (ATCC) were adapted into suspension in a protein-free medium (maintenance media) consisting of 50% HyQ PF (GE Healthcare Life Sciences) and 50% CD CHO (Thermo Fisher Scientific) supplemented with 1 g/L sodium carbonate (Sigma), 6 mM glutamine (Sigma) and 0.1% Pluronic F-68 (Thermo Fisher Scientific).

To develop CHO master clone, the suspension CHO K1 cells were transfected with a pTag vector, wherein the pTag vector comprised an EGFP reporter gene and a zeocin resistant gene linked by an EMCV IRES variant (IRESv18). The EGFP-IRES-Zeo cassette is flanked by FLP recombinase recognition sites, a mutant FRT variant (F3) and the wild type FRT (Fwt). An ATG-lacking puromycin resistant gene was placed downstream of Fwt for future selection of correct cassette exchange. The transfected cells were selected in a medium containing zeocin to generate a stably transfected pool. The stably transfected pool was enriched for high EGFP producing cells by fluorescence-activated cell sorting (FACS). Clones were isolated using limiting dilution and screened for high EGFP expression by fluorescence-activated cell sorting (FACS), and integration of one copy of pTag vector was analyzed by southern blotting. A primary master clone Z2A4 was confirmed to have one copy of pTag. This primary master clone was co-transfected with a pExchange vector containing the hygromycin resistant gene and a vector expressing Flpe. The transfected cells were selected in medium containing hygromycin.

The cells in which GFP gene was replaced with hygromycin resistant gene survived the selection. Limiting dilution was carried out again to identify a master clone Z2A4-18 containing one copy of hygromycin resistant gene. The single gene integration was confirmed by southern blot and targeted locus amplification analysis.

### Cell Culture and Media for Maintenance of CHO K1 Master Clone

The CHO K1 master clone was grown in a protein-free medium (maintenance media) consisting of 50% HyQ PF (GE Healthcare Life Sciences) and 50% CD CHO (Thermo Fisher Scientific) supplemented with 1 g/L sodium carbonate (Sigma), 6 mM glutamine (Sigma) and 0.1% Pluronic F-68 (Thermo Fisher Scientific) in a humidified Kuhner shaker (Adolf Kühner AG) with 8% CO2 at 37° C. Routine subculture was conducted every 3 to 4 days by seeding cells at density of 3×105 cells/mL in 15 mL of fresh medium in 125 mL shake flasks (Corning). Cell density and viability were determined by trypan blue exclusion method on Vi-Cell XR viability analysers (Beckman Coulter).

### Generating Stably Transfected Pools Via Recombinase-Mediated-Cassette-Exchange (RMCE)

The CHO K1 master clone was co-transfected with one or two appropriate targeting vectors and a vector expressing FLPe using Amaxa SG Cell Line 4D-Nucleofector X Kit and program FF-137 (Lonza). In each transfection, 1×10⁷ cells were transfected with 5 μg of targeting plasmid vector and 5 μg of FLPe plasmid vector in circular format. The transfected cells were then re-suspended in 2 mL of maintenance media preloaded in 6-well suspension culture plates (NUNC) and incubated in the static IncuSafe incubators (Sanyo). At 24 hours post-transfection, they were collected by centrifugation (100×g, 5 min) and re-suspended in 15 mL of protein-free maintenance medium in 125 mL shake flasks in the humidified Kuhner shaker (Adolf Kühner AG) with 8% CO₂ at 37° C. Four days later, the transfected cells were subjected to selection in the maintenance media containing Puromycin (InvivoGen) at 20 μg/mL. Selection was continued for two weeks by passaging in the selection medium every 3 to 4 days. Stably transfected cell pools were deemed established when cell viabilities recovered over 95%.

### Characterization of Growth and Productivity of Stable Pools Expressing Antibodies

Stably transfected cell pools expressing antibodies were subjected to 14-day fed-batch production by seeding 30 mL of cultures at viable cell density of 3×10⁵ cells/mL in 50 mL tube spin (TPP) in the humidified Kuhner shaker (Adolf Kühner AG) with 8% CO₂ at 37° C. 3mL of Ex-Cell Advanced CHO Feed 1 (with glucose) (Sigma) were added at day 3, 5, 7, 9 and 11. 400 μL 45% (w/v) D-glucose (Sigma) were added when glucose level was dropped below 2 g/L during fed-batch cultures. Cell density, viability and antibody titer were monitored at day 3, 5, 7, 9, 11 and 14 using the Vi-Cell XR viability analyzer (Beckman Coulter) and an IMMAGE 800 immunochemistry system (Beckman Coulter), respectively. The IMMAGE 800 immunochemistry system utilized anti-human Fc region antibodies for IgG quantification. The specific mAb productivity (qP) in the exponential phase of cultures was calculated as the mAb concentration at day 7 divided by the integrated viable cell density (IVCD) which was determined based on the trapezoidal method.

### Protein A Purification

Purification of the harvested culture supernatant was performed using a Tricorn column packed with MabSelect SuRe on a ÄKTA AVANT equipped with a UV detector at 280 nm and 254 nm. Prior to loading sample, the column was equilibrated using Dulbecco PBS at pH 7.4. The cultures were filtered (0.22 μm) without pH adjustment prior to loading into the column. Elution was done using 100 mM sodium acetate (acetic acid and sodium hydroxide) at pH 3.6. The purified samples were neutralized to pH 6.0 with 1 M Tris base buffer. Flow rate was set at 5.0 mL/min. Ultrafiltration concentrators, Vivaspin 20, 3000 MWCO PES were used to concentrate the purified sample to a concentration greater than 15 mg/mL. Centrifugation was performed using swing bucket at 2200 g and at 4° C.

### SDS PAGE Analysis

Prior to SDS-PAGE separation of the purified mAbs, 4 μg of each purified mAb sample was denatured by boiling in the presence of 25 mM reducing agent DTT (Bio-Rad Laborotories, 161-0611) for reduced gel and absence of reducing agent for non-reducing gel at 95° C. for 10 minutes in the 1XLaemmli buffer [62.5 mM Tris-HCl, pH 6.8, 10.5% glycerol (BDH, 101186), 2% SDS (Bio Rad, 161-0148), 0.01% Bromophenol Blue(PlusOne, 17-1329-01). The reduced and non-reduced denatured mAb protein samples were separated by Bio-Rad Mini-PROTEAN® TGX™ polyacrylamide precast gels (4-15%) for 30 minutes at 200 Volt, and stained with 0.1% Coomassie blue R-250 (Pierce, 20278) in 50% methanol, 10% acetic acid, 40% H₂O (V/V). The gel was destained with 10% methanol and 5% acetic acid and 30% ethanol, and then scanned on Imagescanner III (GE Healthcare).

### Immunofluorescence Staining and Flow Cytometry Analysis

To determine the antibody display levels on cell surface, culture supernatant containing 1×10⁷ cells were collected from the stably transfected pools generated using different dual display and secretion targeting vectors at exponential growth phase. The culture supernatant was then centrifuged at 400 g for 5 minutes to remove the supernatant completely. The cell pellet was re-suspended using 500 μL of cold PBS by pipetting up and down for several times and centrifuged again to remove PBS solution. Subsequently the cells were re-suspended in 500 uL of Anti Human IgG (gamma chain specific)—FITC antibody produced in Goat (Sigma, F0132) diluted 100 times in PBS containing 3% BSA (Bovine Serum Albumin) and then incubated at 4° C. for 30 minutes (on ice) in the dark. Next, the cell solution was centrifuged to remove the solution and washed a few times using PBS. Finally, the cells were re-suspended in 500 μL cold PBS and proceed to flow cytometry analysis on a BD FACSCalibur.

### Experimental Result

### Validation of Master Clone for Integration of One Copy of Gene Per Cell and High-Level Antibody Expression

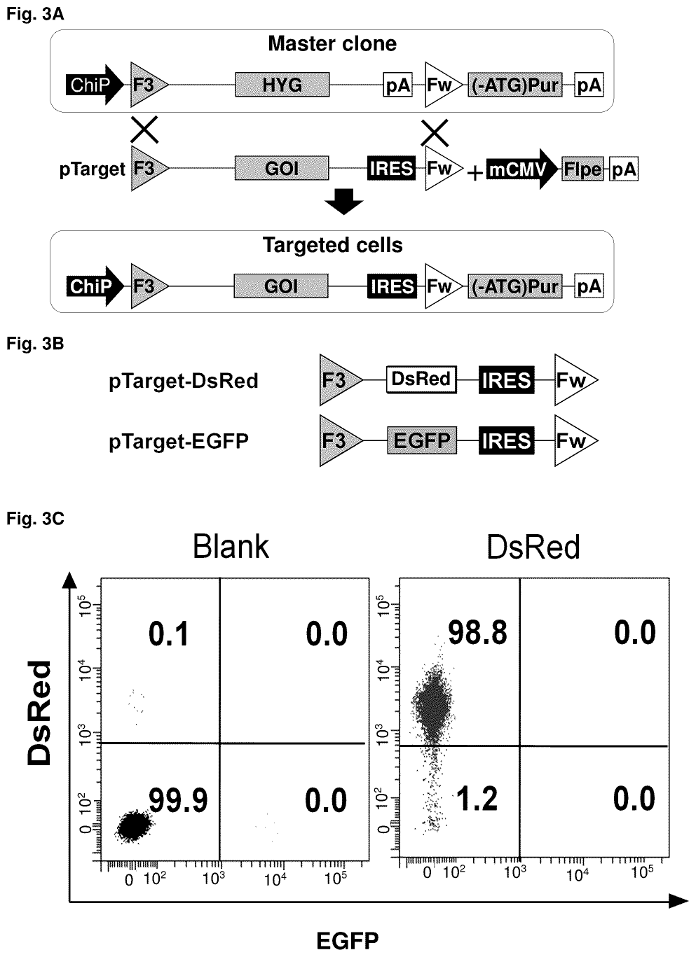

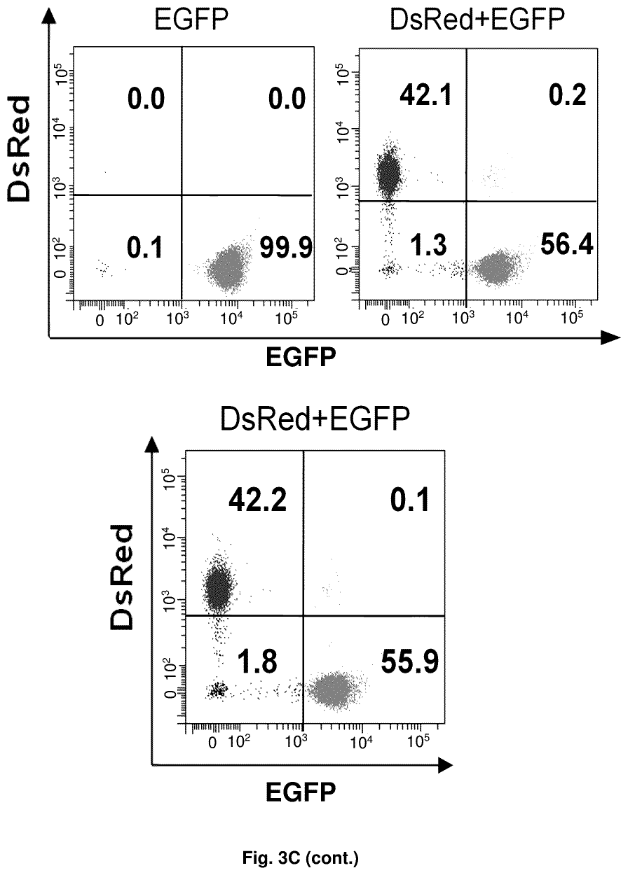

To validate that the generated master clone Z2A4-18 allowed efficient integration of one copy of gene per cell, it was co-transfected with the pTarget-DsRed vector, pTarget-EGFP vector and the vector expressing Flpe (FIG. 3A-3C). Recombinase-mediate cassette exchange (RMCE) enabled replacement of HYG with either DsRed and/or GFP. Selection was done in medium containing puromycin to ensure that only cells with the correct exchange survived. Fluorescence-activated cell sorting (FACS) analysis indicated that the co-transfected pools consisted of 0.1% to 0.2% of cells expressing both DsRed and EGFP, indicating that more than 99% of the cells expressed only either one of the genes.

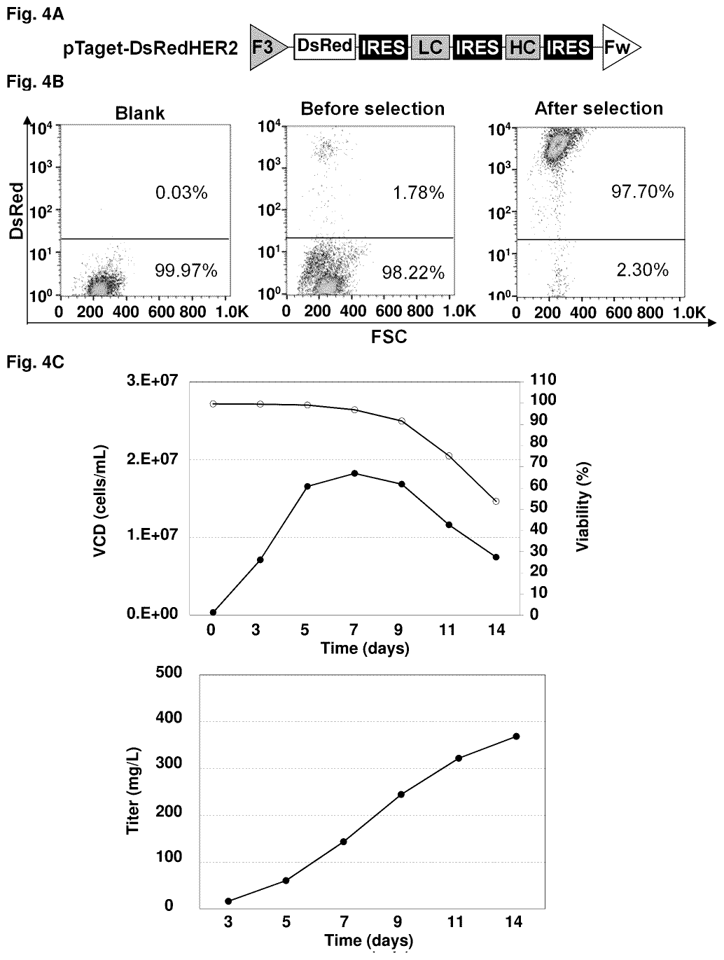

To evaluate the master clone for expressing monoclonal antibodies and exchange efficiency, the master clone was co-transfected with a targeting vector pTarget-DsRedHER2 containing DsRed, LC and HC (FIG. 4A) and the vector expressing Flpe using the recombinase-mediated cassette exchange (RMCE) process as described in FIG. 3A. 1.78% of cells expressing DsRed before selection in medium containing puromycin and 97.70% of cells expressing DsRed after selection (FIG. 4B), indicating that exchange efficiency is around close to 2%. The peak cell densities of the targeted pools after selection reached 1.8E7 cells/mL and the antibody titer at the end of culture was 380 mg/L (FIG. 4C).

### Vector Design for Simultaneous Display and Secretion of Full-Length IgG Antibodies

Next, one set of targeting vectors was designed, wherein the display and secretion of full-length IgG antibodies in CHO cells were tested (FIG. 5A and 5B) through RMCE. The control vector expressed the antibody light chain (LC) and heavy chain (HC) genes in one transcript through the use of internal ribosome entry site (IRES). GPI vector has a similar design as the control vector except that the HC gene is linked to a membrane anchor. Other targeting vectors were designed with the HC gene linked to the membrane anchor through furin cleavage sequence RRKR (SEQ ID NO: 3) alone, 2A peptide alone or RRKR-2A peptide combinations. Furin is a recombinant, ubiquitous subtilisin-like proprotein convertase with a minimal cleavage sequence of R-X-K-R (SEQ ID NO: 1) or R-X-R-R (SEQ ID NO: 2) and the cleavage of proteins occurs in Golgi. 2A peptides have approximately 20 amino acids and “self-cleavage” occurs co-translationally between the last two amino acids, glycine and proline. Many types of 2A peptides have been identified from virus. Different 2A peptides have different cleavage efficiency. The cleavage efficiency of 2A and furin are also affected their flanking amino acids.

The control targeting vector without the membrane anchor had high level secretion of antibodies into the culture medium but very little display of antibodies on the cell surface (FIG. 5C). In contrast, GPI vector had high display levels but no secretion of antibodies. The RRKR-GPI vector showed higher display levels than the GPI vector but no secretion of antibodies. The flanking amino acids from GPI may inhibit the cleavage of furin at RRKR (SEQ ID NO: 3). Linking HC with GPI through all four 2A peptides resulted in both display and secretion at different ratios. The secreted HC polypeptides from these vectors had a bigger size compared to that expressed by the control vector (FIG. 5C). Peptide mapping analysis indicated they were attached with 2A or 2A-GPI residues. Higher secretion and lower display indicated higher cleavage efficiency of a 2A peptide. The results indicated that amongst the different 2A peptides, E2A showed the highest cleavage efficiency followed by T2A, F2A and P2A. Using RRKR-2A to link HC and membrane anchor resulted in higher secretion and lower display for P2A, E2A and T2A but not for E2A. The secretion level from RRKR-P2A-GPI, RRKR-F2A-GPI and RRKR-T2A-GPI had been increased to a level close to the control vector. The HC polypeptides secreted from RRKR-F2A-GPI, RRKR-T2A-GPI and RRKR-T2A-GPI vectors still contained a significant proportion of species attached with 2A or 2A-GPI residues. Only RRKR-P2A-GPI vector produced antibodies without the incorrect species (FIG. 5C). However, the display level from this vector was too low to be separated the stained cells from the blank cells.

### Engineering P2A Peptide and Furin Recognition Sequence for Mediating the Ratios of Membrane Bound to Secretory Antibodies

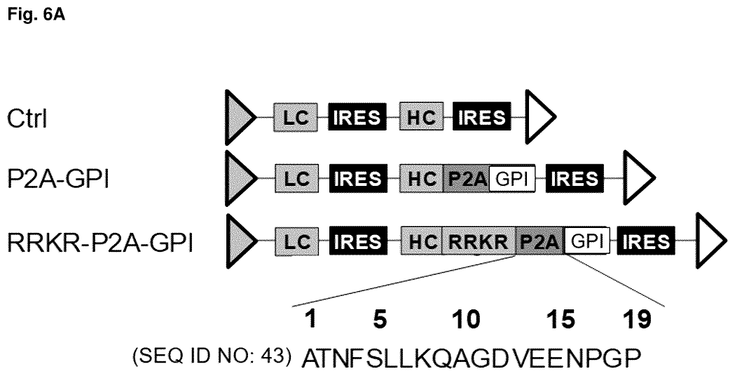

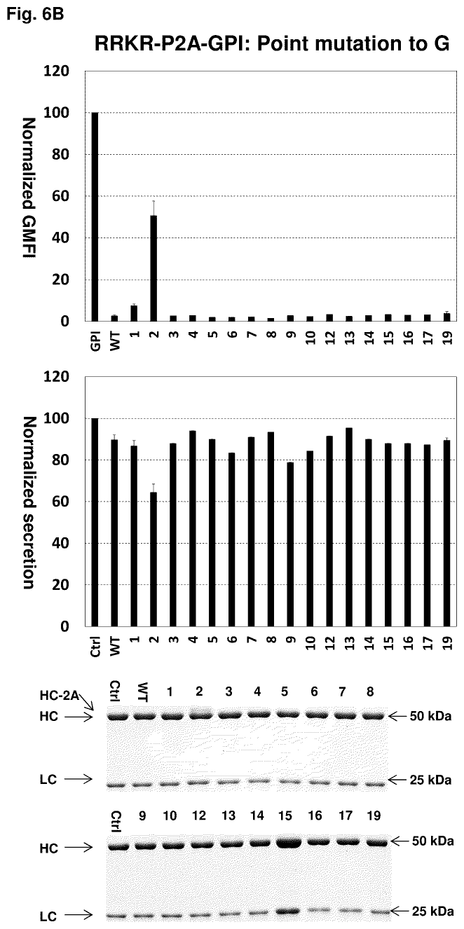

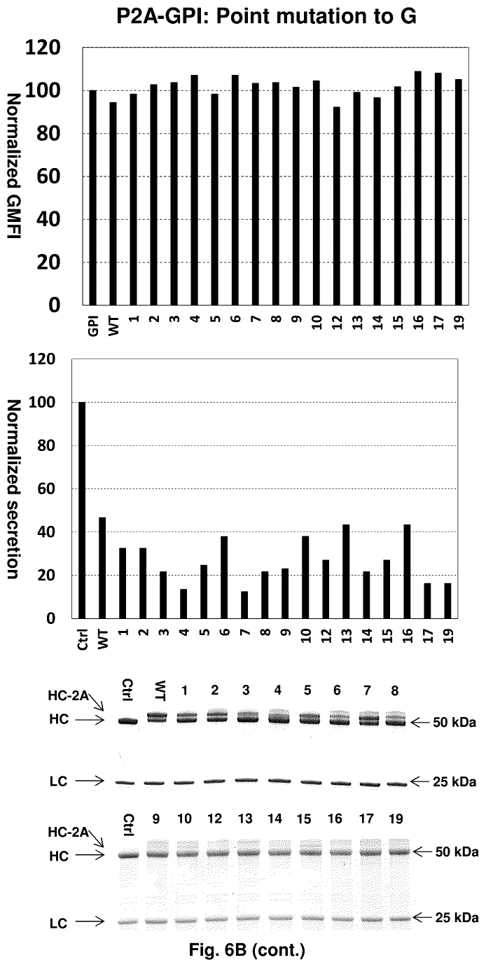

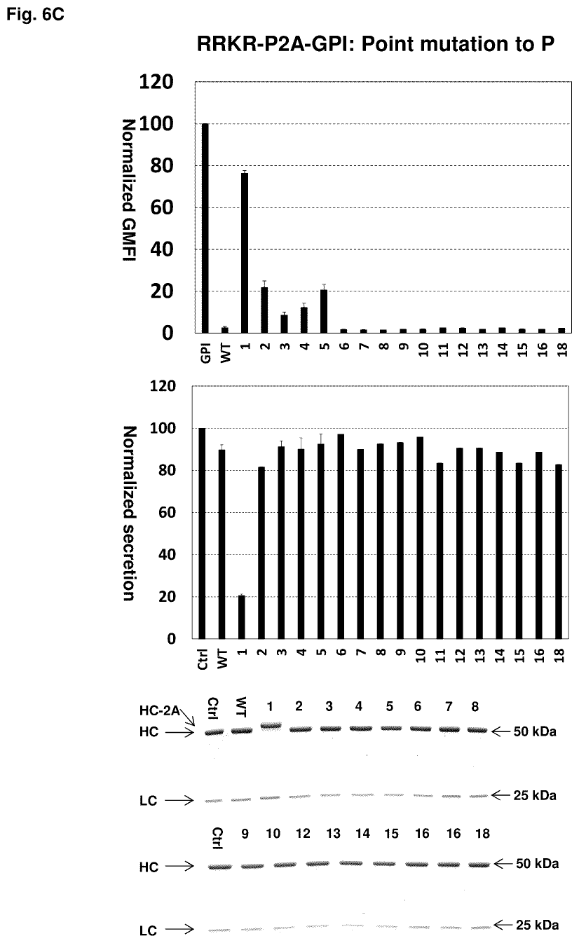

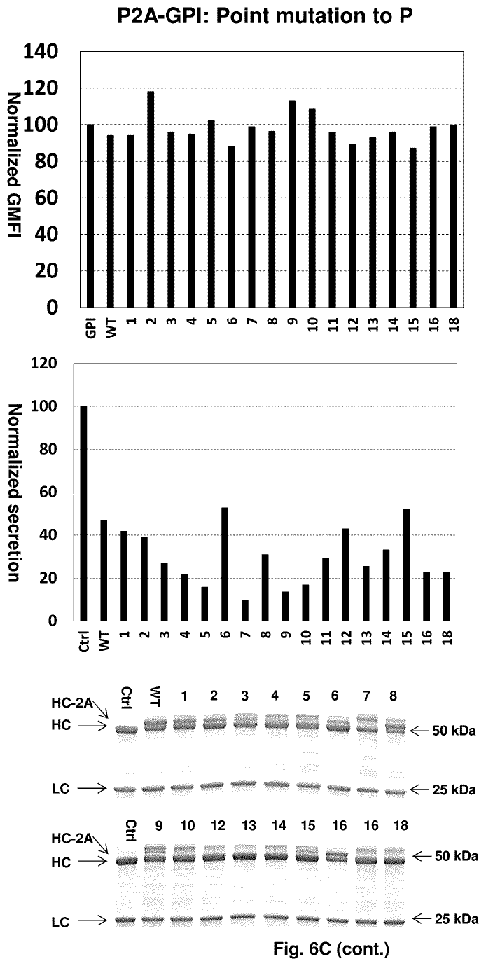

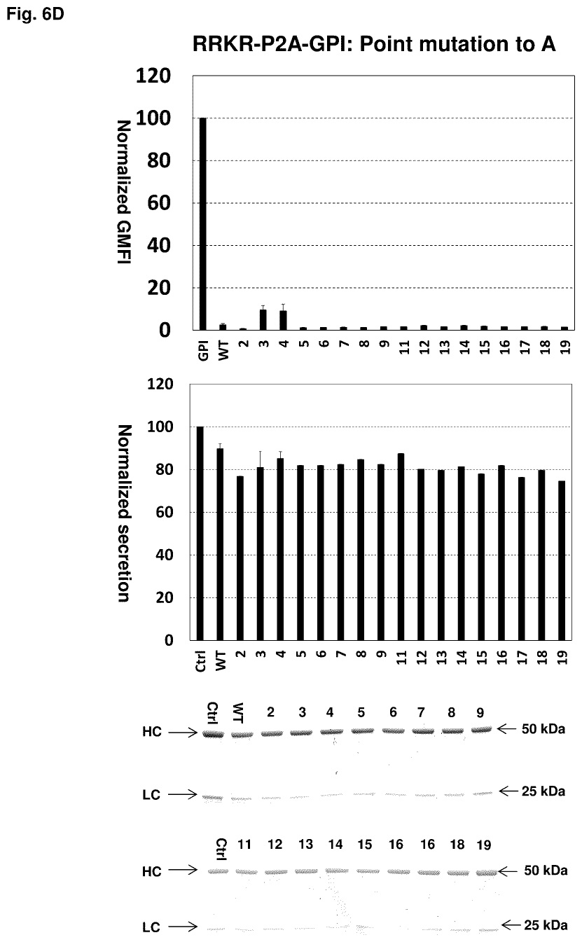

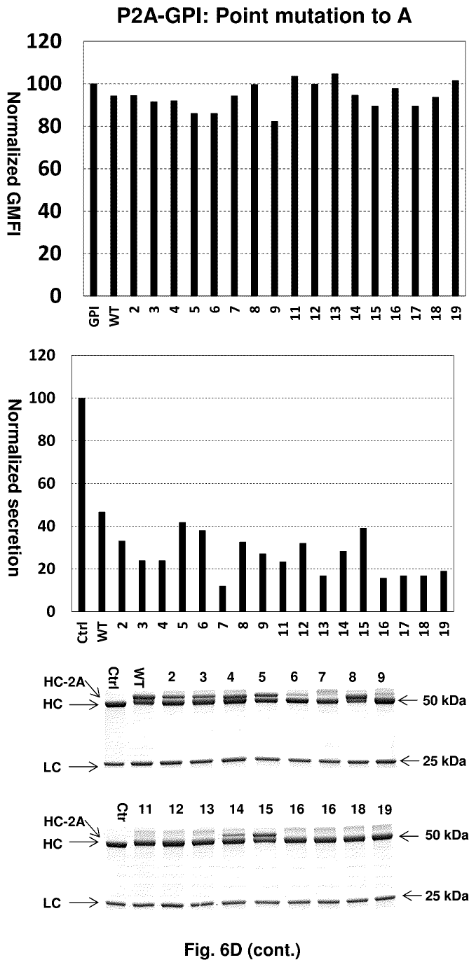

To increase the ratios of display to secretion of antibodies from the RRKR-P2A-GPI vector, point mutations of P2A were created, wherein the point mutations include the individual residue to glycine, proline or alanine (FIG. 6). Proline and glycine influence secondary structure, constraining or providing high flexibility to peptide chains, respectively. In contrast, alanine has little influence on secondary structure. Another set of targeting vector containing P2A with same mutations were tested to understand how the mutations affect the cleavage of efficiency of P2A. Nine point mutations, A1G, T2G, A1P, T2P, N3P, F4P, SSP, N3A and F4A increased the display level from the RRKR-P2A-GPI vectors compared the wild type P2A. The level of expression is provided in Table 2. The increased level varied depending on the mutations. Al P and T2G increased the display level to 80% and 50% of the GPI vector. The secreted products from RRKR-P2A-GPI containing these two mutations contained incorrect product. Peptide mapping analysis indicated the majority of incorrect species were HC polypeptides attached with P2A residues and a minor proportion were attached with P2A-GPI residues. RRKR-P2A-GPI vectors containing other 7 mutations increased the display level ranging from 9% to 20% of the GPI vector. The secreted HC polypeptides from these vectors had correct size. However, peptide mapping analysis indicated a small proportion of species were attached with 2A and 2A-GPI residues. Many point mutations decreased cleavage efficiency of P2A as indicated by the decreased secretion from P2A-GM vectors. However, these mutations did not increase the display from the F-P2A-GM vectors suggesting these mutations affected only the cleavage efficiency of P2A. Cleavage at RRKR (SEQ ID NO: 3) still resulted in removal of GPI from HC and secretion of antibodies. The 9 mutations which increased the display level from the RRKR-P2A-GPI vectors could be due to their effect on the cleavage efficiency at both RRKR (SEQ ID NO: 3) and P2A.

TABLE 2
Levels of membrane bound antigen binding molecule measured by normalised geometric mean
fluorescene intensity (GMFI) values, levels of secreted antigen binding molecule and presence of
expression systems with the relevant point mutations based on FIG. 6.

| | A1G | T2G | A1P | T2P | N3P | F4P | S5P | N3A | F4A |
|---|---|---|---|---|---|---|---|---|---|
| Normalised | 10% | 50% | 75% | 25% | 10% | 15% | 20% | 10% | 10% |
| GMFI | | | | | | | | | |
| Normalised | 90% | 65% | 20% | 85% | 90% | 90% | 90% | 80% | 85% |
| levels of | | | | | | | | | |
| secreted | | | | | | | | | |
| antigen | | | | | | | | | |
| binding | | | | | | | | | |
| molecule | | | | | | | | | |
| Levels of | 100% | 90% | 0 | 100% | 100% | 100% | 100% | 100% | 100% |
| secreted | | | | | | | | | |
| antigen | | | | | | | | | |
| binding | | | | | | | | | |
| molecule | | | | | | | | | |
| Levels of | 0 | 10% | 100% | 0 | 0 | 0 | 0 | 0 | 0 |
| incorrect | | | | | | | | | |
| secreted | | | | | | | | | |
| antigen | | | | | | | | | |
| binding | | | | | | | | | |
| molecule | | | | | | | | | |

The 9 identified variants with point mutations which increased the display level from the F-P2A-GM vectors located at the first to fifth amino acid of P2A. It was indicated that the 5 flanking amino acids downstream of R-X-K-R (SEQ ID NO: 1) or R-X-R-R (SEQ ID NO: 2) were conserved and may play roles in determining the furin cleavage efficiency. To understand how the 9 variants with point mutations affect the furin cleavage efficiency, one set of targeting vectors with the heavy chain (HC) linked with GPI through furin cleavage sequence variants was constructed. These furin cleavage sequence variant-GPI vectors, or variant expression system, comprises the first 5 amino acids from the N-terminus of P2A variants downstream of RRKR (SEQ ID NO: 3). The variant expression system are: RRKR-(ATNFS)-GPI, RRKR-(GTNFS)-GPI, RRKR-(ATNAS)-GPI, RRKR-(ATPFS)-GPI, RRKR-(ATAFS)-GPI, RRKR-(ATNPS)-GPI, RRKR-(ATNFP)-GPI, RRKR-(APNFS)-GPI, RRKR-(AGNFS)-GPI, RRKR-(PTNFS)-GPI. The RRKR-P2A-GPI vectors, or main expression systems containing same mutations were included for comparison. The RRKR-P2A-GPI vectors are RRKR-(ATNFS)P2A-GPI, RRKR-(GTNFS)P2A-GPI, RRKR-(ATNAS)P2A-GPI, RRKR-(ATPFS)P2A-GPI, RRKR-(ATAFS)P2A-GPI, RRKR-(ATNPS)P2A-GPI, RRKR-(ATNFP)P2A-GPI, RRKR-(APNFS)P2A-GPI, RRKR-(AGNFS)P2A-GPI, RRKR-(PTNFS)P2A-GPI (FIG. 7A). All furin cleavage sequence variant-GPI vectors exhibited increased secretion and decreased display compared to the RRKR-GPI vector (FIG. 7B). Different furin cleavage sequence variants controlled the ratios of display to secretion at different levels. The relative changes in secretion and display levels correlated well to the changes from RRKR-P2A-GPI vectors containing same mutations except for RRKR-GTNFS (SEQ ID NO: 62) and RRKR-APNFS (SEQ ID NO: 68). The targeting vectors containing these two furin cleavage sequence variants, RRKR-GTNFS (SEQ ID NO: 62) and RRKR-APNFS (SEQ ID NO: 68) exhibited about 5-fold and 3-fold higher display levels than their corresponding targeting vectors containing RRKR-(GTNFS)P2A and RRKR-(APNFS)P2A. Correspondingly, the secretion level from the targeting vectors containing these two furin cleavage sequence variants dropped compared to their corresponding targeting vectors containing RRKR-(GTNFS)P2A and RRKR-(APNFS)P2A but the magnitude of decrease is less than the level of increased display. Good correlation of changes in the levels of display and secretion between the targeting vectors contain furin cleavage sequence variants and the targeting vectors containing RRKR-P2A with corresponding point mutations in P2A indicated that the point mutations in P2A affected mainly the cleavage efficiency at RRKR rather than at P2A. The discrepancy of changes in the levels display and secretion between the targeting vectors containing RRKR-GTNFS and RRKR-APNFS and their corresponding targeting vectors containing RRKR-(GTNFS)P2A and RRKR-(APNFS)P2A indicate that point mutations of A1G and T2P affected cleavage efficiencies at both RRKR and P2A. The HC polypeptides expressed from the targeting vectors containing RRKR-AGNFS-GPI and RRKR-PTNFS-GPI contained species with molecular weight greater than the control HC polypeptide. Peptide mapping analysis indicated a small proportion of HC polypeptides from RRKR-AGNFS-GPI vector were attached with GPI. Occurrence of these incorrect species in the medium may be resulted from cell death. The other 7 furin cleavage sequence variants gave correct molecular weight of HC polypeptides. Peptide mapping analysis confirmed that all secreted HC polypeptides from the RRKR-GTNFS-GPI vector had correct amino acid sequence.

### Application of Simultaneous Display and Secretion System for Antibody Humanization

The expression system as disclosed herein has different applications. For example, the expression system can be used in antibody discovery, and can replace hybridoma and single B cell cloning to raise antibodies from immunized mice. This allows the rapid identification and production of antibodies from human blood for treatment of infectious disease, such as COVID-19.

The expression systems as disclosed herein can also be used for antibody humanization and affinity maturation, as well as antibody production. The expression system allows for rapid cell line development for producing monoclonal antibodies. This can allow for cell line development for homogenous production of polyclonal antibodies, such as recombinant IVIG for treatment of immunodeficiency disease.

### Discussion

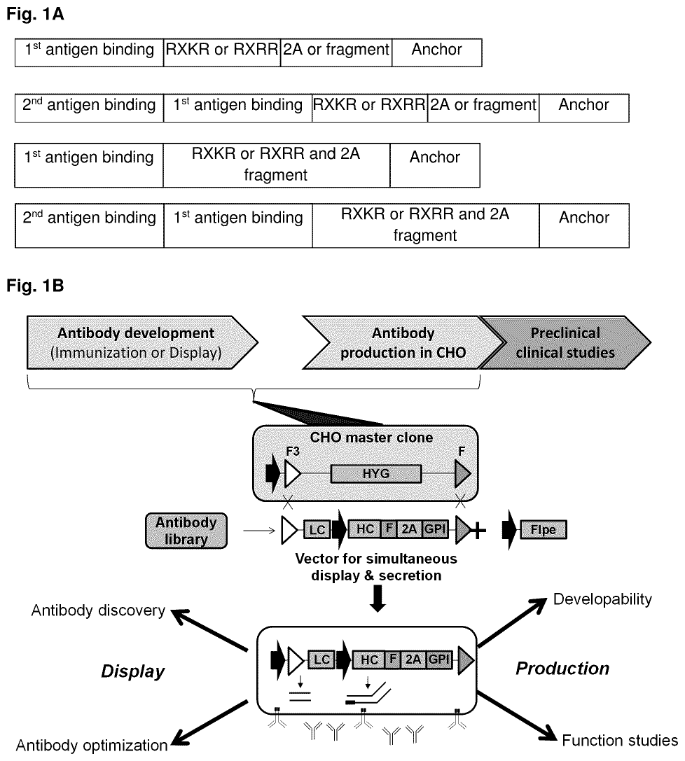

A mammalian expression system is developed to permit simultaneous cell surface display and secretion of the same protein at different ratios (FIGS. 1A and 5A). This system consists of a CHO master clone and one set of targeting expression vectors which allows simultaneous display and secretion of proteins at different ratios. The CHO master clone contains a predetermined integration site which allows site specific integration of one copy of plasmid vector per cell through recombinase-mediated cassette exchange (RMCE). Each targeting vector carries an antibody light chain (LC) gene and a heavy chain (HC) gene linked by an EMCV IRES. The heavy chain (HC) gene is linked with a membrane anchor through a combination of a minimal furin recognition sequence RXKR (SEQ ID NO: 1) or RXRR (SEQ ID NO: 2), and a 2A peptide or fragment thereof. The RRKR-2A linked HC and membrane anchor are translated in one open reading frame.

Cleavage at both the minimal furin recognition sequence and 2A peptide results in the heavy chain (HC) polypeptides without attachment of 2A residues and membrane anchor, which assembles with light chain (LC) polypeptides to form secreted antibodies. Cleavage at 2A but not at RRKR results in secreted incorrect antibodies in which HC polypeptides are attached with 2A residues. Incomplete cleavage at both 2A and the minimal furin recognition sequence results in heavy chain (HC) polypeptides fused with membrane anchor, which assembles with light chain (LC) polypeptides to form membrane-bounded antibodies. Obtaining varied ratios of display to secretion of antibodies requires controlled cleavage efficiency of furin and 2A at different levels.

In addition, the cleavage at the minimal furin recognition sequence needs to be controlled at higher efficiency than at 2A peptide to ensure secreted antibodies without attachment of 2A residues to the C-terminus of HC polypeptide. Many types of 2A peptides have been identified from virus. Different 2A peptides have different cleavage efficiency. The cleavage efficiency of 2A and the minimal furin recognition sequence are also affected by their flanking amino acid sequence. Different combinations of the minimal furin recognition sequence and the different types of 2A peptides were screened for simultaneous display and secretion of antibodies. The results indicated that E2A has the highest cleavage efficiency followed by T2A, F2A and P2A. In one example, the targeting vector containing the RRKR-P2A combination secretes correct antibodies without 2A residues attached to the HC polypeptides. However, the display level from the vector with the wild-type P2A peptide is too low to be used for screening antibody binding affinities. To increase the relative amount of cell surface to the secreted antibody, one set of P2A variants was generated with different cleavage efficiencies by point mutations. It was observed that the first five amino acids of P2A are critical for the cleavage efficiency of both furin and P2A. Nine specific point mutations, A1G, T2G, A1P, T2P, N3P, F4P, S5P, N3A and F4A, which inhibit cleavage of furin and P2A at different efficiencies, increased the display level ranging from 9% to 80% of the control vector which linked HC directly with the membrane anchor. The secreted products from the two targeting vectors containing Al P and T2G respectively, which gave the highest display level of 80% and 50% of the control vector, contain incorrect product with 2A residues attached to the HC polypeptide due to lower cleavage at RRKR than at P2A. All the targeting vectors containing the other point mutations secreted correct antibody products. Point mutations of the first five amino acids to other amino acids or mutation at other sites have no effect on cleavage at both RRKR (SEQ ID NO: 3) and P2A cleavage or only inhibit cleavage of P2A. The targeting vectors containing these mutations did not change the display level as the membrane anchor can still be removed by cleavage at RRKR. To cover a wider range of ratios of display to secretion and ensuring secretion of correct product, a high through screening of a library of the main and variant expression systems in which the first five amino acids of P2A are randomized to identify variants with controlled cleavage efficiencies at RRKR and P2A (FIG. 7A).

The expression system using RRKR-P2A enables secretion of product without attachment of 2A residues to the heavy chain (HC) polypeptides. This expression system also has the advantage of obtaining higher secretion at comparable or even higher display levels than those obtained by using the minimal furin recognition sequence. This is because furin cleavage occurs at Golgi. Attachment of the membrane anchor to the HC polypeptides could result in improper folding at ER which leads to unfolded protein response (UPR) and thus earlier cell death in fed-batch cultures. In contrast, 2A “self-cleavage” occurs co-transnationally. Removing the membrane anchor on some HC polypeptides before entering ER could reduce ER stress and thus enhance the viability and secreted antibody titer in fed-batch cultures.

In contrast to the other expression systems, the expression system of the present disclosure allows simultaneous display and secretion of antibody. Some point mutations of P2A in the expression system of the present disclosure also show increased display levels because these mutations inhibited cleavage of at both RRKR and P2A. By linking the heavy chain (HC) and the membrane anchor through furin cleavage sequence variants (designed by inclusion of the first 5 amino acids of P2A variants), it allows secretion and display at different ratios. Compared to the other expression systems, it has the advantage of avoiding secretion of the incorrect product with 2A residues attached to the heavy chain (HC) polypeptides.

In summary, the inventors have developed a platform technology which provides antibody discovery and production capabilities in one system. Such a platform consists of three key components: 1) a CHO master clone containing a predetermined genomic site that provides stable and high-level gene expression, 2) a targeting vector which allows simultaneous display and secretion of antibodies, and 3) a library consisting of diverse antibodies (FIG. 1B). Discovery of desirable antibodies is achieved by cloning the antibody library into the targeting vector to generate a plasmid library, which is then en masse transfected into the CHO master cells. By utilizing the recombinase-mediated-cassette-exchange (RMCE), each master cell comprises one DNA copy of a distinct antibody integrated into the pre-selected genomic site, creating a CHO cell library expressing mixtures of many different secreted IgGs. With the display function, cells presented antibodies with high binding affinity, high specificity and good manufacturability can be identified from this CHO cell library through FACS-based high throughput sorting. With the secretion function, the identified CHO cells presenting promising antibodies can be directly used as production cell lines to produce enough material for developability assessment and functional studies. As such, this platform provides opportunity to establish a streamlined process for high speed and low-cost development of therapeutic antibodies.

TABLE 3
Details of SEQ ID NOs referenced herein and their corresponding sequences. A
brief description of the sequences is also provided.

| SEQ ID | | |
|---|---|---|
| NO | Sequence | Description |
| 1 | RXKR | Exemplary minimal Furin |
| | | cleavage consensus |
| | | sequence |
| 2 | RXRR | Exemplary minimal Furin |
| | | cleavage consensus |
| | | sequence |
| 3 | RRKR | Exemplary minimal Furin |
| | | cleavage consensus |
| | | sequence |
| 4 | RRRR | Exemplary minimal Furin |
| | | cleavage consensus |
| | | sequence |
| 5 | RSKR | Exemplary minimal Furin |
| | | cleavage consensus |
| | | sequence |
| 6 | RSRR | Exemplary minimal Furin |
| | | cleavage consensus |
| | | sequence |
| 7 | RKKR | Exemplary minimal Furin |
| | | cleavage consensus |
| | | sequence |
| 8 | RKRR | Exemplary minimal Furin |
| | | cleavage consensus |
| | | sequence |
| 9 | RQKR | Exemplary minimal Furin |
| | | cleavage consensus |
| | | sequence |
| 10 | RQRR | Exemplary minimal Furin |
| | | cleavage consensus |
| | | sequence |
| 11 | RTKR | Exemplary minimal Furin |
| | | cleavage consensus |
| | | sequence |
| 12 | RTRR | Exemplary minimal Furin |
| | | cleavage consensus |
| | | sequence |
| 13 | REKR | Exemplary minimal Furin |
| | | cleavage consensus |
| | | sequence |
| 14 | RERR | Exemplary minimal Furin |
| | | cleavage consensus |
| | | sequence |
| 15 | RDKR | Exemplary minimal Furin |
| | | cleavage consensus |
| | | sequence |
| 16 | RDRR | Exemplary minimal Furin |
| | | cleavage consensus |
| | | sequence |
| 17 | RHKR | Exemplary minimal Furin |
| | | cleavage consensus |
| | | sequence |
| 18 | RHRR | Exemplary minimal Furin |
| | | cleavage consensus |
| | | sequence |
| 19 | RFKR | Exemplary minimal Furin |
| | | cleavage consensus |
| | | sequence |
| 20 | RFRR | Exemplary minimal Furin |
| | | cleavage consensus |
| | | sequence |
| 21 | RAKR | Exemplary minimal Furin |
| | | cleavage consensus |
| | | sequence |
| 22 | RARR | Exemplary minimal Furin |
| | | cleavage consensus |
| | | sequence |
| 23 | RNKR | Exemplary minimal Furin |
| | | cleavage consensus |
| | | sequence |
| 24 | RNRR | Exemplary minimal Furin |
| | | cleavage consensus |
| | | sequence |
| 25 | RCKR | Exemplary minimal Furin |
| | | cleavage consensus |
| | | sequence |
| 26 | RCRR | Exemplary minimal Furin |
| | | cleavage consensus |
| | | sequence |
| 27 | RGKR | Exemplary minimal Furin |
| | | cleavage consensus |
| | | sequence |
| 28 | RGRR | Exemplary minimal Furin |
| | | cleavage consensus |
| | | sequence |
| 29 | RIKR | Exemplary minimal Furin |
| | | cleavage consensus |
| | | sequence |
| 30 | RIRR | Exemplary minimal Furin |
| | | cleavage consensus |
| | | sequence |
| 31 | RLKR | Exemplary minimal Furin |
| | | cleavage consensus |
| | | sequence |
| 32 | RLRR | Exemplary minimal Furin |
| | | cleavage consensus |
| | | sequence |
| 33 | RMKR | Exemplary minimal Furin |
| | | cleavage consensus |
| | | sequence |
| 34 | RMRR | Exemplary minimal Furin |
| | | cleavage consensus |
| | | sequence |
| 35 | RPKR | Exemplary minimal Furin |
| | | cleavage consensus |
| | | sequence |
| 36 | RPRR | Exemplary minimal Furin |
| | | cleavage consensus |
| | | sequence |
| 37 | RWKR | Exemplary minimal Furin |
| | | cleavage consensus |
| | | sequence |
| 38 | RWRR | Exemplary minimal Furin |
| | | cleavage consensus |
| | | sequence |
| 39 | RYKR | Exemplary minimal Furin |
| | | cleavage consensus |
| | | sequence |
| 40 | RYRR | Exemplary minimal Furin |
| | | cleavage consensus |
| | | sequence |
| 41 | RVKR | Exemplary minimal Furin |
| | | cleavage consensus |
| | | sequence |
| 42 | RVRR | Exemplary minimal Furin |
| | | cleavage consensus |
| | | sequence |
| 43 | ATNFSLLKQAGDVEENPGP | P2A amino acid sequence |
| | | (wild-type) |
| 44 | APVKQTLNFDLLKLAGDVESNPGP | F2A amino acid sequence |
| | | (wild-type) |
| 45 | QCTNYALLKLAGDVESNPGP | E2A amino acid sequence |
| | | (wild-type) |
| 46 | EGRGSLLTCGDVEENPGP | T2A amino acid sequence |
| | | (wild-type) |
| 47 | GCT ACT AAC TTC AGC CTG CTG AAG CAG | P2A nucleotide sequence |
| | GCT GGA GAC GTG GAG GAG AAC CCT GGG | (wild-type) |
| | CCC | |
| 48 | GCA CCG GTG AAA CAG ACT TTG AAT TTT | F2A nucleotide sequence |
| | GAC CTT CTG AAG TTG GCA GGA GAC GTT | (wild-type) |
| | GAG TCC AAC CCT GGG CCC | |
| 49 | CAG TGT ACT AAT TAT GCT CTC TTG AAA | E2A nucleotide sequence |
| | TTG GCT GGA GAT GTT GAG AGC AAC CCT | (wild-type) |
| | GGG CCC | |
| 50 | GAG GGC AGA GGA AGT CTG CTA ACA TGC | T2A nucleotide sequence |
| | GGT GAC GTC GAG GAG AAT CCT GGG CCC | (wild-type) |
| 51 | ATNFS | Exemplary P2A fragment |
| | | sequence |
| 52 | GTNFS | Exemplary P2A fragment |
| | | sequence with A1G |
| | | mutation |
| 53 | ATNAS | Exemplary P2A fragment |
| | | sequence with F4A mutation |
| 54 | ATPFS | Exemplary P2A fragment |
| | | sequence with N3P mutation |
| 55 | ATAFS | Exemplary P2A fragment |
| | | sequence with N3A |
| | | mutation |
| 56 | ATNPS | Exemplary P2A fragment |
| | | sequence with F4P mutation |
| 57 | ATNFP | Exemplary P2A fragment |
| | | sequence with S5P mutation |
| 58 | APNFS | Exemplary P2A fragment |
| | | sequence with T2P mutation |
| 59 | AGNES | Exemplary P2A fragment |
| | | sequence with T2G mutation |
| 60 | PTNFS | Exemplary P2A fragment |
| | | sequence with A1P mutation |
| 61 | RRKRATNFS | Exemplary Furin RRKR and |
| | | P2A fragment sequence |
| 62 | RRKRGTNFS | Exemplary Furin RRKR and |
| | | P2A fragment sequence with |
| | | A1G mutation |
| 63 | RRKRATNAS | Exemplary Furin RRKR and |
| | | P2A fragment sequence with |
| | | F4A mutation |
| 64 | RRKRATPFS | Exemplary Furin RRKR and |
| | | P2A fragment sequence with |
| | | N3P mutation |
| 65 | RRKRATAFS | Exemplary Furin RRKR and |
| | | P2A fragment sequence with |
| | | N3A mutation |
| 66 | RRKRATNPS | Exemplary Furin RRKR and |
| | | P2A fragment sequence with |
| | | F4P mutation |
| 67 | RRKRATNFP | Exemplary Furin RRKR and |
| | | P2A fragment sequence with |
| | | S5P mutation |
| 68 | RRKRAPNFS | Exemplary Furin RRKR and |
| | | P2A fragment sequence with |
| | | T2P mutation |
| 69 | RRKRAGNFS | Exemplary Furin RRKR and |
| | | P2A fragment sequence with |
| | | T2G mutation |
| 70 | RRKRPTNFS | Exemplary Furin RRKR and |
| | | P2A fragment sequence with |
| | | A1P mutation |
| 71 | RRRRATNFS | Exemplary Furin RRRR and |
| | | P2A fragment sequence |
| 72 | RRRRGTNFS | Exemplary Furin RRRR and |
| | | P2A fragment sequence with |
| | | A1G mutation |
| 73 | RRRRATNAS | Exemplary Furin RRRR and |
| | | P2A fragment sequence with |
| | | F4A mutation |
| 74 | RRRRATPFS | Exemplary Furin RRRR and |
| | | P2A fragment sequence with |
| | | N3P mutation |
| 75 | RRRRATAFS | Exemplary Furin RRRR and |
| | | P2A fragment sequence with |
| | | N3A mutation |
| 76 | RRRRATNPS | Exemplary Furin RRKR and |
| | | P2A fragment sequence with |
| | | F4P mutation |
| 77 | RRRRATNFP | Exemplary Furin RRRR and |
| | | P2A fragment sequence with |
| | | S5P mutation |
| 78 | RRRRAPNFS | Exemplary Furin RRRR and |
| | | P2A fragment sequence with |
| | | T2P mutation |
| 79 | RRRRAGNES | Exemplary Furin RRRR and |
| | | P2A fragment sequence with |
| | | T2G mutation |
| 80 | RRRRPTNFS | Exemplary Furin RRRR and |
| | | P2A fragment sequence with |
| | | A1P mutation |
| 81 | RXKRATNFS | Exemplary Furin RXKR and |
| | | P2A fragment sequence |
| 82 | RXKRGTNFS | Exemplary Furin RXKR and |
| | | P2A fragment sequence with |
| | | A1G mutation |
| 83 | RXKRATNAS | Exemplary Furin RXKR and |
| | | P2A fragment sequence with |
| | | F4A mutation |
| 84 | RXKRATPFS | Exemplary Furin RXKR and |
| | | P2A fragment sequence with |
| | | N3P mutation |
| 85 | RXKRATAFS | Exemplary Furin RXKR and |
| | | P2A fragment sequence with |
| | | N3A mutation |
| 86 | RXKRATNPS | Exemplary Furin RXKR and |
| | | P2A fragment sequence with |
| | | F4P mutation |
| 87 | RXKRATNFP | Exemplary Furin RXKR and |
| | | P2A fragment sequence with |
| | | S5P mutation |
| 88 | RXKRAPNFS | Exemplary Furin RXKR and |
| | | P2A fragment sequence with |
| | | T2P mutation |
| 89 | RXKRAGNFS | Exemplary Furin RXKR and |
| | | P2A fragment sequence with |
| | | T2G mutation |
| 90 | RXKRPTNFS | Exemplary Furin RXKR and |
| | | P2A fragment sequence with |
| | | A1P mutation |
| 91 | RXRRATNFS | Exemplary Furin RXRR and |
| | | P2A fragment sequence |
| 92 | RXRRGTNFS | Exemplary Furin RXRR and |
| | | P2A fragment sequence with |
| | | A1G mutation |
| 93 | RXRRATNAS | Exemplary Furin RXRR and |
| | | P2A fragment sequence with |
| | | F4A mutation |
| 94 | RXRRATPFS | Exemplary Furin RXRR and |
| | | P2A fragment sequence with |
| | | N3P mutation |
| 95 | RXRRATAFS | Exemplary Furin RXRR and |
| | | P2A fragment sequence with |
| | | N3A mutation |
| 96 | RXRRATNPS | Exemplary Furin RXKR and |
| | | P2A fragment sequence with |
| | | F4P mutation |
| 97 | RXRRATNFP | Exemplary Furin RXRR and |
| | | P2A fragment sequence with |
| | | S5P mutation |
| 98 | RXRRAPNFS | Exemplary Furin RXRR and |
| | | P2A fragment sequence with |
| | | T2P mutation |
| 99 | RXRRAGNFS | Exemplary Furin RXRR and |
| | | P2A fragment sequence with |
| | | T2G mutation |
| 100 | RXRRPTNFS | Exemplary Furin RXRR and |
| | | P2A fragment sequence with |
| | | A1P mutation |
| 101 | GTNFSLLKQAGDVEENPGP | Exemplary P2A sequence |
| | | with A1G mutation |
| 102 | ATNASLLKQAGDVEENPGP | Exemplary P2A sequence |
| | | with F4A mutation |
| 103 | ATPFSLLKQAGDVEENPGP | Exemplary P2A sequence |
| | | with N3P mutation |
| 104 | ATAFSLLKQAGDVEENPGP | Exemplary P2A sequence |
| | | with N3A mutation |
| 105 | ATNPSLLKQAGDVEENPGP | Exemplary P2A sequence |
| | | with F4P mutation |
| 106 | ATNFPLLKQAGDVEENPGP | Exemplary P2A sequence |
| | | with S5P mutation |
| 107 | APNFSLLKQAGDVEENPGP | Exemplary P2A sequence |
| | | with T2P mutation |
| 108 | AGNFSLLKQAGDVEENPGP | Exemplary P2A sequence |
| | | with T2G mutation |
| 109 | PTNFSLLKQAGDVEENPGP | Exemplary P2A sequence |
| | | with A1P mutation |
| 110 | RRKRATNFSLLKQAGDVEENPGP | Exemplary Furin RRKR and |
| | | P2A sequence |
| 111 | RRKRGTNFSLLKQAGDVEENPGP | Exemplary Furin RRKR and |
| | | P2A sequence with A1G |
| | | mutation |
| 112 | RRKRATNASLLKQAGDVEENPGP | Exemplary Furin RRKR and |
| | | P2A sequence with F4A |
| | | mutation |
| 113 | RRKRATPFSLLKQAGDVEENPGP | Exemplary Furin RRKR and |
| | | P2A sequence with N3P |
| | | mutation |
| 114 | RRKRATAFSLLKQAGDVEENPGP | Exemplary Furin RRKR and |
| | | P2A sequence with N3A |
| | | mutation |
| 115 | RRKRATNPSLLKQAGDVEENPGP | Exemplary Furin RRKR and |
| | | P2A sequence with F4P |
| | | mutation |
| 116 | RRKRATNFPLLKQAGDVEENPGP | Exemplary Furin RRKR and |
| | | P2A sequence with S5P |
| | | mutation |
| 117 | RRKRAPNFSLLKQAGDVEENPGP | Exemplary Furin RRKR and |
| | | P2A sequence with T2P |
| | | mutation |
| 118 | RRKRAGNFSLLKQAGDVEENPGP | Exemplary Furin RRKR and |
| | | P2A sequence with T2G |
| | | mutation |
| 119 | RRKRPTNFSLLKQAGDVEENPGP | Exemplary Furin RRKR and |
| | | P2A sequence with A1P |
| | | mutation |
| 120 | RRRRATNFSLLKQAGDVEENPGP | Exemplary Furin RRRR and |
| | | P2A sequence |
| 121 | RRRRGTNFSLLKQAGDVEENPGP | Exemplary Furin RRRR and |
| | | P2A sequence with A1G |
| | | mutation |
| 122 | RRRRATNASLLKQAGDVEENPGP | Exemplary Furin RRRR and |
| | | P2A sequence with F4A |
| | | mutation |
| 123 | RRRRATPFSLLKQAGDVEENPGP | Exemplary Furin RRRR and |
| | | P2A sequence with N3P |
| | | mutation |
| 124 | RRRRATAFSLLKQAGDVEENPGP | Exemplary Furin RRRR and |
| | | P2A sequence with N3A |
| | | mutation |
| 125 | RRRRATNPSLLKQAGDVEENPGP | Exemplary Furin RRKR and |
| | | P2A sequence with F4P |
| | | mutation |
| 126 | RRRRATNFPLLKQAGDVEENPGP | Exemplary Furin RRRR and |
| | | P2A sequence with S5P |
| | | mutation |
| 127 | RRRRAPNFSLLKQAGDVEENPGP | Exemplary Furin RRRR and |
| | | P2A sequence with T2P |
| | | mutation |
| 128 | RRRRAGNFSLLKQAGDVEENPGP | Exemplary Furin RRRR and |
| | | P2A sequence with T2G |
| | | mutation |
| 129 | RRRRPTNFSLLKQAGDVEENPGP | Exemplary Furin RRRR and |
| | | P2A sequence with A1P |
| | | mutation |
| 130 | RXKRATNFSLLKQAGDVEENPGP | Exemplary Furin RXKR and |
| | | P2A sequence |
| 131 | RXKRGTNFSLLKQAGDVEENPGP | Exemplary Furin RXKR and |
| | | P2A sequence with A1G |
| | | mutation |
| 132 | RXKRATNASLLKQAGDVEENPGP | Exemplary Furin RXKR and |
| | | P2A sequence with F4A |
| | | mutation |
| 133 | RXKRATPFSLLKQAGDVEENPGP | Exemplary Furin RXKR and |
| | | P2A sequence with N3P |
| | | mutation |
| 134 | RXKRATAFSLLKQAGDVEENPGP | Exemplary Furin RXKR and |
| | | P2A sequence with N3A |
| | | mutation |
| 135 | RXKRATNPSLLKQAGDVEENPGP | Exemplary Furin RXKR and |
| | | P2A sequence with F4P |
| | | mutation |
| 136 | RXKRATNFPLLKQAGDVEENPGP | Exemplary Furin RXKR and |
| | | P2A sequence with S5P |
| | | mutation |
| 137 | RXKRAPNFSLLKQAGDVEENPGP | Exemplary Furin RXKR and |
| | | P2A sequence with T2P |
| | | mutation |
| 138 | RXKRAGNFSLLKQAGDVEENPGP | Exemplary Furin RXKR and |
| | | P2A sequence with T2G |
| | | mutation |
| 139 | RXKRPTNFSLLKQAGDVEENPGP | Exemplary Furin RXKR and |
| | | P2A sequence with A1P |
| | | mutation |
| 140 | RXRRATNFSLLKQAGDVEENPGP | Exemplary Furin RXRR and |
| | | P2A sequence |
| 141 | RXRRGTNFSLLKQAGDVEENPGP | Exemplary Furin RXRR and |
| | | P2A sequence with A1G |
| | | mutation |
| 142 | RXRRATNASLLKQAGDVEENPGP | Exemplary Furin RXRR and |
| | | P2A sequence with F4A |
| | | mutation |
| 143 | RXRRATPFSLLKQAGDVEENPGP | Exemplary Furin RXRR and |
| | | P2A sequence with N3P |
| | | mutation |
| 144 | RXRRATAFSLLKQAGDVEENPGP | Exemplary Furin RXRR and |
| | | P2A sequence with N3A |
| | | mutation |
| 145 | RXRRATNPSLLKQAGDVEENPGP | Exemplary Furin RXKR and |
| | | P2A sequence with F4P |
| | | mutation |
| 146 | RXRRATNFPLLKQAGDVEENPGP | Exemplary Furin RXRR and |
| | | P2A sequence with S5P |
| | | mutation |
| 147 | RXRRAPNFSLLKQAGDVEENPGP | Exemplary Furin RXRR and |
| | | P2A sequence with T2P |
| | | mutation |
| 148 | RXRRAGNFSLLKQAGDVEENPGP | Exemplary Furin RXRR and |
| | | P2A sequence with T2G |
| | | mutation |
| 149 | RXRRPTNFSLLKQAGDVEENPGP | Exemplary Furin RXRR and |
| | | P2A sequence with A1P |
| | | mutation |
| 150 | RRKRATNFSLLKQAGDVEENPGPPNKGSGTTSG | Amino acid sequence of |
| | TTRLLSGHTCFTLTGLLGTLVTMGLLT | RRKR-P2A-GPI section |
| | | in expression system |
| 151 | RRKRATNFSLLKQAGDVEENPGPPNKGSGTTSG | Amino acid sequence of |
| | TTRLLSGHTCFTLTGLLGTLVTMGLLT | RRKR-(ATNFS)P2A-GPI |
| | | section in expression system |
| 152 | RRKRATNFSPNKGSGTTSGTTRLLSGHTCFTLT | Amino acid sequence of |
| | GLLGTLVTMGLLT | RRKR-ATNFS-GPI section |
| | | in expression system |
| 153 | RRKRGTNFSLLKQAGDVEENPGPPNKGSGTTSG | Amino acid sequence of |
| | TTRLLSGHTCFTLTGLLGTLVTMGLLT | RRKR-(GTNFS)P2A-GPI |
| | | section in expression system |
| | | (comprises A1G mutation in |
| | | P2A polypeptide) |
| 154 | RRKRGTNFSPNKGSGTTSGTTRLLSGHTCFTLT | Amino acid sequence of |
| | GLLGTLVTMGLLT | RRKR-GTNFS-GPI section |
| | | in expression system |
| | | (comprises A1G mutation in |
| | | P2A polypeptide fragment) |
| 155 | RRKRATNASLLKQAGDVEENPGPPNKGSGTTS | Amino acid sequence of |
| | GTTRLLSGHTCFTLTGLLGTLVTMGLLT | RRKR-(ATNAS)P2A-GPI |
| | | section in expression system |
| | | (comprises F4A mutation in |
| | | P2A polypeptide) |
| 156 | RRKRATNASPNKGSGTTSGTTRLLSGHTCFTLT | Amino acid sequence of |
| | GLLGTLVTMGLLT | RRKR-ATNAS-GPI section |
| | | in expression system |
| | | (comprises F4A mutation in |
| | | P2A polypeptide fragment) |
| 157 | RRKRATPFSLLKQAGDVEENPGPPNKGSGTTSG | Amino acid sequence of |
| | TTRLLSGHTCFTLTGLLGTLVTMGLLT | RRKR-(ATPFS)P2A-GPI |
| | | section in expression system |
| | | (comprises N3P mutation in |
| | | P2A polypeptide) |
| 158 | RRKRATPFSPNKGSGTTSGTTRLLSGHTCFTLTG | Amino acid sequence of |
| | LLGTLVTMGLLT | RRKR-ATPFS-GPI section |
| | | in expression system |
| | | (comprises N3P mutation in |
| | | P2A polypeptide fragment) |
| 159 | RRKRATAFSLLKQAGDVEENPGPPNKGSGTTSG | Amino acid sequence of |
| | TTRLLSGHTCFTLTGLLGTLVTMGLLT | RRKR-(ATAFS)P2A-GPI |
| | | section in expression system |
| | | (comprises N3A mutation in |
| | | P2A polypeptide) |
| 160 | RRKRATAFSPNKGSGTTSGTTRLLSGHTCFTLT | Amino acid sequence of |
| | GLLGTLVTMGLLT | RRKR-ATAFS-GPI section |
| | | in expression system |
| | | (comprises N3A mutation in |
| | | P2A polypeptide fragment) |
| 161 | RRKRATNPSLLKQAGDVEENPGPPNKGSGTTSG | Amino acid sequence of |
| | TTRLLSGHTCFTLTGLLGTLVTMGLLT | RRKR-(ATNPS)P2A-GPI |
| | | section in expression system |
| | | (comprises F4P mutation in |
| | | P2A polypeptide) |
| 162 | RRKRATNPSPNKGSGTTSGTTRLLSGHTCFTLT | Amino acid sequence of |
| | GLLGTLVTMGLLT | RRKR-ATNPS-GPI section |
| | | in expression system |
| | | (comprises F4P mutation in |
| | | P2A polypeptide fragment |
| 163 | RRKRATNFPLLKQAGDVEENPGPPNKGSGTTSG | Amino acid sequence of |
| | TTRLLSGHTCFTLTGLLGTLVTMGLLT | RRKR-(ATNFP)P2A-GPI |
| | | section in expression system |
| | | (comprises S5P mutation in |
| | | P2A polypeptide) |
| 164 | RRKRATNFPPNKGSGTTSGTTRLLSGHTCFTLT | Amino acid sequence of |
| | GLLGTLVTMGLLT | RRKR-ATNFP-GPI section |
| | | in expression system |
| | | (comprises S5P mutation in |
| | | P2A polypeptide fragment) |
| 165 | RRKRAPNFSLLKQAGDVEENPGPPNKGSGTTSG | Amino acid sequence of |
| | TTRLLSGHTCFTLTGLLGTLVTMGLLT | RRKR-(APNFS)P2A-GPI |
| | | section in expression system |
| | | (comprises T2P mutation in |
| | | P2A polypeptide) |
| 166 | RRKRAPNFSPNKGSGTTSGTTRLLSGHTCFTLT | Amino acid sequence of |
| | GLLGTLVTMGLLT | RRKR-APNFS-GPI section |
| | | in expression system |
| | | (comprises T2P mutation in |
| | | P2A polypeptide fragment) |
| 167 | RRKRAGNFSLLKQAGDVEENPGPPNKGSGTTS | Amino acid sequence of |
| | GTTRLLSGHTCFTLTGLLGTLVTMGLLT | RRKR-(AGNFS)P2A-GPI |
| | | section in expression system |
| | | (comprises T2G mutation in |
| | | P2A polypeptide) |
| 168 | RRKRAGNFSPNKGSGTTSGTTRLLSGHTCFTLT | Amino acid sequence of |
| | GLLGTLVTMGLLT | RRKR-AGNFS-GPI section |
| | | in expression system |
| | | (comprises T2G mutation in |
| | | P2A polypeptide fragment) |
| 169 | RRKRPTNFSLLKQAGDVEENPGPPNKGSGTTSG | Amino acid sequence of |
| | TTRLLSGHTCFTLTGLLGTLVTMGLLT | RRKR-(PTNFS)P2A-GPI |
| | | section in expression system |
| | | (comprises A1P mutation in |
| | | P2A polypeptide) |
| 170 | RRKRPTNFSPNKGSGTTSGTTRLLSGHTCFTLTG | Amino acid sequence of |
| | LLGTLVTMGLLT | RRKR-PTNFS-GPI section |
| | | in expression system |
| | | (comprises A1P mutation in |
| | | P2A polypeptide fragment) |
| 171 | RRKRAPVKQTLNFDLLKLAGDVESNPGP | RRKR-F2A (wild-type) |
| | | amino acid sequence |
| 172 | RRKRQCTNYALLKLAGDVESNPGP | RRKR-E2A (wild-type) |
| | | amino acid sequence |
| 173 | RRKREGRGSLLTCGDVEENPGP | RRKR-T2A (wild-type) |
| | | amino acid sequence |
| 174 | RRKRATNFSLLKQAGDVEENPGP | RRKR-P2A (wild-type) |
| | | amino acid sequence |
| 175 | PNKGSGTTSGTTRLLSGHTCFTLTGLLGTLVTM | GPI amino acid sequence |
| | GLLT | |
| 176 | CGG AGA AAG CGC GCA CCG GTG AAA CAG | RRKR-F2A (wild-type) |
| | ACT TTG AAT TTT GAC CTT CTG | nucleotide sequence |
| | AAG TTG GCA GGA GAC GTT GAG TCC AAC | |
| | CCT GGG CCC | |
| 177 | CGG AGA AAG CGC CAG TGT ACT AAT TAT | RRKR-E2A (wild-type) |
| | GCT CTC TTG AAA TTG GCT GGA | nucleotide sequence |
| | GAT GTT GAG AGC AAC CCT GGG CCC | |
| 178 | CGG AGA AAG CGC GAG GGC AGA GGA AGT | RRKR-T2A (wild-type) |
| | CTG CTA ACA TGC GGT GAC GTC | nucleotide sequence |
| | GAG GAG AAT CCT GGG CCC | |
| 179 | CGG AGA AAG CGC GCT ACT AAC TTC AGC | RRKR-P2A (wild-type) |
| | CTG CTG AAG CAG GCT GGA GAC | nucleotide sequence |
| | GTG GAG GAG AAC CCT GGG CCC | |
| 180 | CCA AAT AAA GGA AGT GGA ACC ACT TCA | GPI nucleotide sequence |
| | GGT ACT ACC CGT CTT CTA TCT | |
| | GGG CAC ACG TGT TTC ACG TTG ACA GGT | |
| | TTG CTT GGG ACG CTA GTA ACC | |
| | ATG GGC TTG CTG ACT | |
| 181 | CCCCTCTCCCTCCCCCCCCCCTAACGTTACTG | IRES nucleotide sequence |
| | GCCGAAGCCGCTTGGAATAAGGCCGGTGTGC | |
| | GTTTGTCTATATGTTATTTTCCACCATATTGCC | |
| | GTCTTTTGGCAATGTGAGGGCCCGGAAACCT | |
| | GGCCCTGTCTTCTTGACGAGCATTCCTAGGGG | |
| | TCTTTCCCCTCTCGCCAAAGGAATGCAAGGTC | |
| | TGTTGAATGTCGTGAAGGAAGCAGTTCCTCTG | |
| | GAAGCTTCTTGAAGACAAACAACGTCTGTAG | |
| | CGACCCTTTGCAGGCAGCGGAACCCCCCACC | |
| | TGGCGACAGGTGCCTCTGCGGCCAAAAGCCA | |
| | CGTGTATAAGATACACCTGCAAAGGCGGCAC | |
| | AACCCCAGTGCCACGTTGTGAGTTGGATAGTT | |
| | GTGGAAAGAGTCAAATGGCTCTCCTCAAGCG | |
| | TATTCAACAAGGGGCTGAAGGATGCCCAGAA | |
| | GGTACCCCATTGTATGGGATCTGATCTGGGGC | |
| | CTCGGTACACATGCTTTACATGTGTTTAGTCG | |
| | AGGTTAAAAAAACGTCTAGGCCCCCCGAACC | |
| | ACGGGGACGTGGTTTTCCTTTGAAAACGCGTA | |
| | TGATAATATGGCCACAACC | |
| 182 | AGTCAATGGGAAAAACCCATTGGAGCCAAGT | ChiP nucleotide sequence |
| | ACACTGACTCAATAGGGACTTTCCATTGGGTT | |
| | TTGCCCAGTACATAAGGTCAATAGGGGGTGA | |
| | GTCAACAGGAAAGTCCCATTGGAGCCAAGTA | |
| | CATTGAGTCAATAGGGACTTTCCAATGGGTTT | |
| | TGCCCAGTACATAAGGTCAATGGGAGGTAAG | |
| | CCAATGGGTTTTTCCCATTACTGGCACGTATA | |
| | CTGAGTCATTAGGGACTTTCCAATGGGTTTTG | |
| | CCCAGTACATAAGGTCAATAGGGGTGAATCA | |
| | ACAGGAAAGTCCCATTGGAGCCAAGTACACT | |
| | GAGTCAATAGGGACTTTCCATTGGGTTTTGCC | |
| | CAGTACAAAAGGTCAATAGGGGGTGAGTCAA | |
| | TGGGTTTTTCCCATTATTGGCACGTACATAAG | |
| | GTCAATAGGGGTGACTAGTCATGGTGATGCG | |
| | GTTTTGGCAGTACATCAATGGGCGTGGATAG | |
| | CGGTTTGACTCACGGGGATTTCCAAGTCTCCA | |
| | CCCCATTGACGTCAATGGGAGTTTGTTTTGGC | |
| | ACCAAAATCAACGGGACTTTCCAAAATGTCG | |
| | TAACAACTCCGCCCCATTGACGCAAATGGGC | |
| | GGTAGGCGTGTACGGTGGGAGGTCTATATAA | |
| | GCAGAGCTCGTTTAGTGAACCGTCAGATCGC | |
| | CTGGAGACGCCATCCACGCTGTTTTGACCTCC | |
| | ATAGAAGACACCGGGACCGATCCAGCCTCCG | |
| | CGGCCGGGAACGGTGCATTGGAACGCGGATT | |
| | CCCCGTGCCAAGAGTGACGTAAGTACCGCCT | |
| | ATAGACTCTATAGGCACACCCCTTTGGCTCTT | |
| | ATGCATGCTATACTGTTTTTGGCTTGGGGCCT | |
| | ATACACCCCCGCTCCTTATGCTATAGGTGATG | |
| | GTATAGCTTAGCCTATAGGTGTGGGTTATTGA | |
| | CCATTATTGACCACTCCCCTATTGGTGACGAT | |
| | ACTTTCCATTACTAATCCATAACATGGCTCTT | |
| | TGCCACAACTATCTCTATTGGCTATATGCCAA | |
| | TACTCTGTCCTTCAGAGACTGACACGGACTCT | |
| | GTATTTTTACAGGATGGGGTCCCATTTATTAT | |
| | TTACAAATTCACATATACAACAACGCCGTCCC | |
| | CCGTGCCCGCAGTTTTTATTAAACATAGCGTG | |
| | GGATCTCCACGCGAATCTCGGGTACGTGTTCC | |
| | GGACATGGGCTCTTCTCCGGTAGCGGCGGAG | |
| | CTTCCACATCCGAGCCCTGGTCCCATGCCTCC | |
| | AGCGGCTCATGGTCGCTCGGCAGCTCCTTGCT | |
| | CCTAACAGTGGAGGCCAGACTTAGGCACAGC | |
| | ACAATGCCCACCACCACCAGTGTGCCGCACA | |
| | AGGCCGTGGCGGTAGGGTATGTGTCTGAAAA | |
| | TGAGCTCGGAGATTGGGCTCGCACCGTGACG | |
| | CAGATGGAAGACTTAAGGCAGCGGCAGAAGA | |
| | AGATGCAGGCAGCTGAGTTGTTGTATTCTGAT | |
| | AAGAGTCAGAGGTAACTCCCGTTGCGGTGCT | |
| | GTTAACGGTGGAGGGCAGTGTAGTCTGAGCA | |
| | GTACTCGTTGCTGCCGCGCGCGCCACCAGAC | |
| | ATAATAGCTGACAGACTAACAGACTGTTCCTT | |
| | TCCATGGGTCTTTTCTGCAGTCACCGTC | |
| 183 | RXKRPTNFSPNKGSGTTSGTTRLLSGHTCFTLTG | Amino acid sequence of |
| | LLGTLVTMGLLT | RXKR-PTNFS-GPI |
| | | (comprises A1P mutation in |
| | | P2A polypeptide fragment) |
| 184 | RXKRGTNFSPNKGSGTTSGTTRLLSGHTCF | Amino acid sequence of |
| | TLTGLLGTLVTMGLLT | RXKR-GTNFS-GPI |
| | | (comprises A1G mutation |
| | | in P2A polypeptide |
| | | fragment) |
| 185 | RXKRAGNFSPNKGSGTTSGTTRLLSGHTCF | Amino acid sequence of |
| | TLTGLLGTLVTMGLLT | RXKR-AGNFS-GPI |
| | | (comprises T2G mutation |
| | | in P2A polypeptide |
| | | fragment) |
| 186 | RXKRAPNFSPNKGSGTTSGTTRLLSGHTCFT | Amino acid sequence of |
| | LTGLLGTLVTMGLLT | RXKR-APNFS-GPI |
| | | (comprises T2P mutation |
| | | in P2A polypeptide |
| | | fragment) |
| 187 | RXKRPTNFSLLKQAGDVEENPGPPNKGSGT | Amino acid sequence of |
| | TSGTTRLLSGHTCFTLTGLLGTLVTMGLLT | RXKR-(PTNFS)P2A-GPI |
| | | (comprises A1P mutation |
| | | in P2A polypeptide) |
| 188 | RXKRGTNFSLLKQAGDVEENPGPPNKGSGT | Amino acid sequence of |
| | TSGTTRLLSGHTCFTLTGLLGTLVTMGLLT | RXKR-(GTNFS)P2A-GPI |
| | | (comprises A1G mutation |
| | | in P2A polypeptide) |
| 189 | RXKRAGNFSLLKQAGDVEENPGPPNKGSG | Amino acid sequence of |
| | TTSGTTRLLSGHTCFTLTGLLGTLVTMGLLT | RXKR-(AGNFS)P2A- |
| | | GPI (comprises T2G |
| | | mutation in P2A |
| | | polypeptide) |
| 190 | RXKRAPNFSLLKQAGDVEENPGPPNKGSGT | Amino acid sequence of |
| | TSGTTRLLSGHTCFTLTGLLGTLVTMGLLT | RXKR-(APNFS)P2A-GPI |
| | | (comprises T2P mutation |
| | | in P2A polypeptide) |
| 191 | RXRRPTNFSPNKGSGTTSGTTRLLSGHTCFT | Amino acid sequence of |
| | LTGLLGTLVTMGLLT | RXRR-PTNFS-GPI |
| | | (comprises A1P mutation |
| | | in P2A polypeptide |
| | | fragment) |
| 192 | RXRRGTNFSPNKGSGTTSGTTRLLSGHTCFT | Amino acid sequence of |
| | LTGLLGTLVTMGLLT | RXRR-GTNFS-GPI |
| | | (comprises A1G mutation |
| | | in P2A polypeptide |
| | | fragment) |
| 193 | RXRRAGNFSPNKGSGTTSGTTRLLSGHTCF | Amino acid sequence of |
| | TLTGLLGTLVTMGLLT | RXRR-AGNFS-GPI |
| | | (comprises T2G mutation |
| | | in P2A polypeptide |
| | | fragment) |
| 194 | RXRRAPNFSPNKGSGTTSGTTRLLSGHTCFT | Amino acid sequence of |
| | LTGLLGTLVTMGLLT | RXRR-APNFS-GPI |
| | | (comprises T2P mutation |
| | | in P2A polypeptide |
| | | fragment) |
| 195 | RXRRPTNFSLLKQAGDVEENPGPPNKGSGT | Amino acid sequence of |
| | TSGTTRLLSGHTCFTLTGLLGTLVTMGLLT | RXRR-(PTNFS)P2A-GPI |
| | | (comprises A1P mutation |
| | | in P2A polypeptide) |
| 196 | RXRRGTNFSLLKQAGDVEENPGPPNKGSGT | Amino acid sequence of |
| | TSGTTRLLSGHTCFTLTGLLGTLVTMGLLT | RXRR-(GTNFS)P2A-GPI |
| | | (comprises A1G mutation |
| | | in P2A polypeptide) |
| 197 | RXRRAGNFSLLKQAGDVEENPGPPNKGSGT | Amino acid sequence of |
| | TSGTTRLLSGHTCFTLTGLLGTLVTMGLLT | RXRR-(AGNFS)P2A- |
| | | (comprises T2G mutation |
| | | in P2A polypeptide) |
| 198 | RXRRAPNFSLLKQAGDVEENPGPPNKGSGT | Amino acid sequence of |
| | TSGTTRLLSGHTCFTLTGLLGTLVTMGLLT | RXRR-(APNFS)P2A-GPI |
| | | (comprises T2P mutation |
| | | in P2A polypeptide) |
| 199 | RRRRPTNFSPNKGSGTTSGTTRLLSGHTCFT | Amino acid sequence of |
| | LTGLLGTLVTMGLLT | RRRR-PTNFS-GPI |
| | | (comprises A1P mutation |
| | | in P2A polypeptide |
| | | fragment) |
| 200 | RRRRGTNFSPNKGSGTTSGTTRLLSGHTCFT | Amino acid sequence of |
| | LTGLLGTLVTMGLLT | RRRR-GTNFS-GPI |
| | | (comprises A1G mutation |
| | | in P2A polypeptide |
| | | fragment) |
| 201 | RRRRAGNESPNKGSGTTSGTTRLLSGHTCF | Amino acid sequence of |
| | TLTGLLGTLVTMGLLT | RRRR-AGNFS-GPI |
| | | (comprises T2G mutation |
| | | in P2A polypeptide |
| | | fragment) |
| 202 | RRRRAPNFSPNKGSGTTSGTTRLLSGHTCFT | Amino acid sequence of |
| | LTGLLGTLVTMGLLT | RRRR-APNFS-GPI |
| | | (comprises T2P mutation |
| | | in P2A polypeptide |
| | | fragment) |
| 203 | RRRRPTNFSLLKQAGDVEENPGPPNKGSGT | Amino acid sequence of |
| | TSGTTRLLSGHTCFTLTGLLGTLVTMGLLT | RRRR-(PTNFS)P2A-GPI |
| | | (comprises A1P mutation |
| | | in P2A polypeptide) |
| 204 | RRRRGTNFSLLKQAGDVEENPGPPNKGSGT | Amino acid sequence of |
| | TSGTTRLLSGHTCFTLTGLLGTLVTMGLLT | RRRR-(GTNFS)P2A-GPI |
| | | (comprises A1G mutation |
| | | in P2A polypeptide) |
| 205 | RRRRAGNESLLKQAGDVEENPGPPNKGSGT | Amino acid sequence of |
| | TSGTTRLLSGHTCFTLTGLLGTLVTMGLLT | RRRR-(AGNFS)P2A-GPI |
| | | (comprises T2G mutation |
| | | in P2A polypeptide) |
| 206 | RRRRAPNFSLLKQAGDVEENPGPPNKGSGT | Amino acid sequence of |
| | TSGTTRLLSGHTCFTLTGLLGTLVTMGLLT | RRRR-(APNFS)P2A-GPI |
| | | (comprises T2P mutation |
| | | in P2A polypeptide) |

1. An expression system for an antigen binding molecule, wherein the antigen binding molecule is either secretable or membrane-bound, comprising:
molecule;
a first antigen binding polynucleotide encoding a first part of the antigen binding
a cleavage polynucleotide encoding a cleavage site comprising a Furin consensus sequence RXKR (SEQ ID NO: 1) or RXRR (SEQ ID NO: 2), and a 2A polypeptide fragment thereof;
an anchor polynucleotide encoding a membrane anchor polypeptide;
wherein the 2A polypeptide fragment thereof comprises one or more mutations in any one of the amino acid residue to control the cleavage efficiencies of the cleavage site to modulate ratio of production of the secretable antigen binding molecule versus the membrane-bound antigen binding molecule;
wherein the cleavage polynucleotide is in between the first antigen binding polynucleotide and the anchor polynucleotide;
wherein when the cleavage site is cleaved, the secretable antigen binding molecule comprising the first part of the antigen binding molecule is released;
wherein when the cleavage site is not cleaved, the membrane-bound antigen binding molecule comprising the first part of the antigen binding molecule, the Furin consensus sequence RXKR (SEQ ID NO: 1) or RXRR (SEQ ID NO: 2), the 2A polypeptide fragment thereof, and the membrane anchor polypeptide, is released.

2. The expression system of claim 1, further comprising a second antigen binding polynucleotide encoding a second part of the antigen binding molecule.

3. The expression system of claim 2, wherein the 2A polypeptide fragment thereof comprises one or more mutations in amino acid residue 1, 2, 3, 4, or 5.

4. The expression system of claim 1, wherein the 2A polypeptide fragment thereof is selected from a group consisting of a P2A, F2A, E2A, and T2A fragment thereof.

5. The expression system of claim 1, wherein the amino acid is mutated to glycine, proline, or alanine.

6. The expression system of claim 1, wherein the one or more mutations is selected from the group consisting of A1P, A1G, T2G, T2P, N3P, F4P, S5P, N3A, and F4A.

7. The expression system of claim 1, wherein the membrane anchor polypeptide is glycophospholipid transmembrane domain (GPI), platelet-derived growth factor receptor (PDGFR) beta chain transmembrane domain (PTM), or immunoglobulin C2-type extracellular-transmembrane-cytosolic domains of murin B7-1 antigen.

8. An expression system for an antigen binding molecule, wherein the antigen binding molecule is either secretable or membrane-bound, comprising:
a first antigen binding polynucleotide encoding a first part of the antigen binding molecule;
a first cleavage polynucleotide encoding a first cleavage site, wherein the first cleavage site is a minimal Furin cleavage consensus sequence RXKR (SEQ ID NO: 1) or RXRR (SEQ ID NO: 2);
a second cleavage polynucleotide encoding a self-processing second cleavage site, wherein the self-processing second cleavage site is a 2A polypeptide or a fragment thereof;
an anchor polynucleotide encoding a membrane anchor polypeptide;
wherein the 2A polypeptide or the fragment thereof comprises one or more mutations in any one of the amino acid residue to control the cleavage efficiencies of the first cleavage site and the second cleavage site to modulate ratio of production of the secretable antigen binding molecule versus the membrane-bound antigen binding molecule;
wherein the first and second cleavage polynucleotides are in between the first antigen binding polynucleotide and the anchor polynucleotide;
wherein when the first cleavage site is cleaved, the secretable antigen binding molecule comprising the first part of the antigen binding molecule is released;
wherein when the first and second cleavage sites are not cleaved, the membrane-bound antigen binding molecule comprising the first part of the antigen binding molecule, the minimal Furin cleavage consensus sequence RXKR (SEQ ID NO: 1) or RXRR (SEQ ID NO: 2), the 2A polypeptide or a fragment thereof, and the membrane anchor polypeptide, is released.

9. The expression system of claim 8, further comprising a second antigen binding polynucleotide encoding a second part of the antigen binding molecule.

10. The expression system of claim 8, wherein the 2A polypeptide or a fragment thereof comprises one or more mutations in amino acid residue 1, 2, 3, 4, or 5.

11. The expression system of claim 8, wherein the 2A polypeptide or a fragment thereof is selected from a group consisting of P2A, F2A, E2A and T2A, or a fragment thereof.

12. The expression system of claim 8, wherein the amino acid is mutated to glycine, proline, or alanine.

13. The expression system of claim 8, wherein the one or more mutations is selected from the group consisting of A1P, A1G, T2G, T2P, N3P, F4P, S5P, N3A, and F4A.

14. The expression system of claim 8, wherein the membrane anchor polypeptide is glycophospholipid transmembrane domain (GPI), platelet-derived growth factor receptor (PDGFR) beta chain transmembrane domain (PTM), or immunoglobulin C2-type extracellular-transmembrane-cytosolic domains of murin B7-1 antigen.

15-17. (canceled)

18. A method for detecting the presence of one or more secreted antibodies and/or one or more surface-bound antibodies in a sample, the method comprising:
providing an expression system of claim 1;
delivering said expression system to one or more target cells, wherein said target cell transcribes said expression system, wherein once transcribed, said cleavage site is cleaved in a first plurality of first part of the antigen binding molecule, so that said first plurality of first part of the antigen binding molecule do not comprise said membrane anchor polypeptide, and are thereby secreted by said target cell, and wherein said cleavage sites are not cleaved in a second plurality of first part of the antigen binding molecule, so that said second plurality of first part of the antigen binding molecule comprise said membrane anchor polypeptide, and are thereby bound to the surface of said target cell; and
detecting the presence or absence of said first plurality of first part of the antigen binding molecule secreted by said target cell and/or detecting a quantity of said second plurality of first part of the antigen binding molecule on the surface of said target cell.

19. A method for detecting the presence of one or more secreted antibodies and/or one or more surface-bound antibodies in a sample, the method comprising:
providing an expression system of claim 8;
delivering said expression system to one or more target cells, wherein said target cell transcribes said expression system, wherein once transcribed, said first cleavage site is cleaved in a first plurality of first part of the antigen binding molecule, so that said first plurality of first part of the antigen binding molecule do not comprise said membrane anchor polypeptide, and are thereby secreted by said target cell, and wherein said first and second cleavage sites are not cleaved in a second plurality of first part of the antigen binding molecule, so that said second plurality of first part of the antigen binding molecule comprise said membrane anchor polypeptide, and are thereby bound to the surface of said target cell; and
detecting the presence or absence of said first plurality of first part of the antigen binding molecule secreted by said target cell and/or detecting a quantity of said second plurality of first part of the antigen binding molecule on the surface of said target cell.

20. (canceled)
